(19) United States
(12) Patent Application Publication
(10) Pub. No.: US 2025/0281595 A1
(43) Pub. Date: Sep. 11, 2025

(54) NOVEL INFLUENZA ANTIGENS

(71) Applicant: GLAXOSMITHKLINE BIOLOGICALS SA, Rixensart (BE)

(72) Inventors: Normand BLAIS, Rixensart (BE); Lionel SACCONNAY, Rixensart (BE); Ventzislav Bojidarov VASSILEV, Rixensart (BE)

(73) Assignee: GLAXOSMITHKLINE BIOLOGICALS SA, Rixensart (BE)

(21) Appl. No.: 18/571,966

(22) PCT Filed: Jun. 24, 2022
(86) PCT No.: PCT/EP2022/067299

§ 371 Date: Dec. 19, 2023

(30) Foreign Application Priority Data
Jun. 28, 2021 (EP) 21182184.8
Nov. 22, 2021 (GB) 2116787.9

(51) Int. Cl. A61K 39/145; A61P 37/04; C07K 14/005

(52) U.S. Cl. CPC A61K 39/145; A61P 37/04; C07K 14/005; C12N 2760/16222; C12N 2760/16234; C12N 2760/16271

(57) ABSTRACT

Recombinant influenza B strain haemagglutinin antigens, polynucleotides encoding the antigens and immunogenic compositions comprising the antigens and polynucleotides.

### CROSS-REFERENCE TO RELATED APPLICATIONS

This Application is the § 371 U.S. National Phase entry of Int'l App No. PCT/EP2022/067299 filed Jun. 24, 2022, which claims priority to Application No. EP 21182184.8, filed in the European Patent Office Jun. 28, 2021, and to Application No. GB 2116787.9, filed Nov. 22, 2021, all of which are incorporated herein by reference in their entireties.

### INCORPORATION-BY-REFERENCE OF SEQUENCE LISTING

The instant application contains a Sequence Listing which has been submitted electronically in ASCII format and is incorporated herein by reference in its entirety. Said ASCII copy, created on Jun. 26, 2024, is named VB67134US01_SL.txt and is 449,682 bytes in size.

### TECHNICAL FIELD

The present invention relates to novel influenza virus antigens, nucleotide sequences encoding them, novel immunogenic or vaccine compositions, and uses of and methods for producing the antigens and compositions. In particular, the invention relates to modified forms of influenza haemagglutinin (HA) from influenza B strain, nucleotide sequences encoding them, and their use in vaccine compositions for the prevention of influenza virus B infections.

### BACKGROUND OF THE INVENTION

Influenza viruses have a significant impact on global public health, causing millions of cases of severe illness each year, thousands of deaths, and considerable economic losses. Influenza viruses belong to the family Orthomyxoviridae, a family that represents enveloped viruses, the genome of which comprises segmented, negative, single-strand RNA. Influenza viruses are divided into three main types: influenza A virus, influenza B virus and influenza C virus. Influenza A and B are the types that are most clinically relevant to humans and are responsible for the flu season each year. Influenza type C infections generally cause mild illness and are not thought to cause human flu epidemics.

Influenza strains are classified according to host species of origin, geographic site and year of isolation, serial number and for influenza A, by serological properties of subtypes of the two predominant surface glycoproteins HA and neuraminidase (NA). It is these surface proteins, particularly HA, that determine the antigenic specificity of the influenza subtypes or lineages.

Influenza A and influenza B diverged from each other around 2000 years ago and have structural similarities but a low sequence identity in the HA (Ni et al, Biochemistry, 2014, 53:846-854). Influenza B virus was first isolated in 1940, and since the 1980s two genetic lineages have been identified based on the antigenic properties of the HA: B/Victoria/2/87 (B/Vic) and

B/Yamagata/16/88 (B/Yam). Members of these two lineages receive their names based on the similarity of their HA1 to that of the B/Victoria/2/87 and B/Yamagata/16/88 strains, respectively (J. M. Chen et al, Arch. Virol 2007; 152 (2): 415-22).

Influenza A viruses evolve and undergo antigenic variability continuously. Although influenza B virus strains generally evolve more slowly in terms of genetic and antigenic properties than A strains, influenza B virus undergoes sustained antigenic drift.

Vaccination plays a critical role in controlling influenza epidemics and pandemics. Because of the antigenic variability, annual vaccinations are required to provide immunity against the influenza viruses that are in circulation. These are predicted based on viral surveillance data. Selection of the appropriate vaccine strains presents many challenges and frequently results in sub-optimal protection. Both influenza B virus lineages have co-circulated in recent years, but each influenza season, one of these lineages tends to predominate over the other, with fluctuations dependent on the year and region. Current seasonal influenza vaccines are trivalent (TIV), containing two A strain and one B strain virus, or quadrivalent (QIV), containing two A and two B strain viruses. QIVs contain both a B/Victoria and a B/Yamagata influenza B strain and TIVs contain one influenza B strain, from either the Victoria or Yamagata lineage. TIVs can provide some cross-protection against alternate-lineage B strains in adults (van de Witte et al, Vaccine, 2018, 36:6030-6038). Nevertheless, QIVs are advantageous compared to TIVs in terms of avoiding mismatch with circulating strains, and accounting for co-circulation of B strain lineages. Although influenza B virus infects all age groups, it causes substantially higher disease burdens in the very young and the elderly and the impact of mismatch of vaccine strain with B circulating strain has been shown to be more profound in children. It is recognised that influenza B strains could cause significant morbidity and mortality worldwide, and significantly impact adolescents and school children.

The immune response to the current vaccines is largely directed against the highly variable HA. HA is a trimeric protein in which each monomer contains two polypeptide chains, HA1 and HA2, linked by a disulphide bond and anchored in the virus envelope by a C-terminus transmembrane domain. Each monomer is initially expressed as inactive HA0 which is subsequently cleaved by host proteases into HA1 and HA2 subunits which are linked via a disulphide bond to form a metastable prefusion state HA. The triggering event for conversion of HA from prefusion to postfusion conformation has been linked to pH change (drop) upon viral uptake/endocytosis leading to membrane fusion and virus internalization. Although there are conserved features shared between influenza A and influenza B virus in the conformational transition between pre and postfusion conformation, there are substantial differences that influence the detailed mechanisms of this process (Ni et al, 2014).

HA can be divided functionally into two domains, the globular head and the stalk or stem. The globular head is composed of part of HA1 while the stalk or stem structure is composed of the N and C-terminal fragments of HA1 and all of HA2 (Hai et al, J. Virol, 2012 86 (10): 5774-5781). The transmembrane domain and cytosolic tail are also part of HA2. The HA globular head is the main target for antibodies against influenza virus but is also highly variable and subject to constant antigenic drift. In contrast the HA stem is highly conserved and experiences little antigenic drift, but is not very immunogenic.

There remains a need for an influenza vaccine that is not restricted by inherent strain specificity, and that could provide protection against heterologous strains of influenza. In particular, it would be of great interest to have a vaccine that protects against an array of influenza strains that includes recent and evolving seasonal strains, and possible seasonal influenza strains of the future. Various approaches have been taken in attempting to provide a “universal” influenza A vaccine that protects individuals from heterologous strains, for example more recently using the conserved stem portion of HA (Yassine et al, Nature Medicine, 2015, 21 (9): 1065-1070). However, little attention has so far been paid to influenza B strains.

### SUMMARY OF THE INVENTION

It has been found that by optimising the coiled coil, such as by making amino acid substitutions in the coiled coil region of influenza B strain HA, a trimeric HA antigen is obtained which could potentially elicit a broad immune response against a number of different influenza B strains to provide an enhanced B strain vaccine. Moreover, it has been found that HA stem is highly conserved across B strains, both within and between lineages, such that an engineered HA stem-containing antigen from one B strain may be expected to be immuno-protective against other B strains, either from the same lineage or a different lineage or both. It has further been found that recombinant influenza B strain HA ectodomain expressed as a fusion with a heterologous trimerization domain, is surprisingly stable following removal of the trimerization domain, such that recombinant influenza B strain HA ectodomain without a trimerization domain can potentially be employed in an immunogenic composition.

In one aspect there is provided a recombinant influenza B strain haemagglutinin (HA) antigen in trimeric form, comprising one or more mutations (e.g. deletions and/or or point mutations).

In another aspect there is provided an isolated polynucleotide encoding the recombinant HA.

In another aspect there is provided an immunogenic composition comprising a recombinant influenza B strain HA antigen or polynucleotide as described above and a pharmaceutically acceptable carrier.

In another aspect there is provided the immunogenic composition described herein for use in prevention of and/or vaccination against influenza B strain infection or disease.

In another aspect there is provided the immunogenic composition for use in the prevention of and/or vaccination against influenza infection or disease caused by at least one different influenza B strain, which may be a strain from the same or a different B strain lineage from the HA lineage to which the HA ectodomain antigen is derived.

In another aspect there is provided a method for producing a recombinant HA as described above, comprising expressing a polynucleotide as described above in a suitable cell expression system, in particular a eukaryotic cell, such as a mammalian cell, e.g. a human cell such as HEK293T cell, non-human mammalian cell such as a CHO cell, or an insect cell, optionally further comprising purifying/isolating the recombinant HA from the cell.

In another aspect there is provided a method for prevention of and/or vaccination against influenza B strain infection or disease, comprising the administration of an antigen or polynucleotide or immunogenic composition as described above to a person in need thereof, such as a person identified as being at risk of influenza virus infection or disease.

In another aspect there is provided an immunogenic composition comprising a recombinant influenza B strain HA stem antigen, or polynucleotide encoding it, for use in the prevention of and/or vaccination against influenza infection or disease caused by at least one different influenza B strain, which may be a strain from the same or a different B strain lineage compared to the lineage from which the HA antigen is derived.

In another aspect there is provided a method of generating an immune response against influenza B strain, comprising administering a recombinant influenza B strain HA antigen or polynucleotide or immunogenic composition described herein, to a human subject.

In another embodiment there is provided the use of a recombinant influenza B strain HA antigen or polynucleotide described herein, in the manufacture of an immunogenic composition for generating an immune response against influenza B strain in a human subject.

In another aspect there is provided an immunogenic composition comprising a recombinant influenza B strain HA ectodomain antigen obtained by expressing an influenza B strain HA ectodomain fused to a trimerization domain followed by subsequent removal of the trimerization domain. The ectodomain may comprise stabilising mutations as described herein, or it may be the ectodomain of a wild type influenza B HA.

### BRIEF DESCRIPTION OF THE SEQUENCES

SEQ ID NO: 1 Full length HA sequence from B Victoria strain B/Washington/02/2019 (Wash19).

SEQ ID NO: 2 Flu018 stem only construct—B/Washington/02/2019 HA sequence with mutations (deletions in this case) and added linkers shown in FIG. 3; linker found between ectodomain and transmembrane sequence in wildtype removed (see sequence alignment in FIG. 1); added foldon and poly histidine tail.

SEQ ID NO: 3 Flu029 stem only construct—B/Washington/02/2019 sequence, details as for SEQ ID NO: 2, with specific mutations (deletions) shown in FIG. 3.

SEQ ID NO: 4 Flu035 stem only construct—B/Washington/02/2019 sequence, details as for SEQ ID NO: 2, with specific mutations (deletions) shown in FIG. 3.

SEQ ID NO: 5 Flu036 stem only construct—B/Washington/02/2019 sequence, details as for SEQ ID NO: 2, with specific mutations (deletions) shown in FIG. 3.

SEQ ID NO: 6 Flu075 stem only construct—B/Washington/02/2019 sequence, details as for SEQ ID NO: 2, with specific mutations (deletions and amino acid substitutions) shown in FIG. 3.

SEQ ID NO: 7 Flu077 stem only construct—B/Washington/02/2019 sequence, details as for SEQ ID NO: 2, with specific mutations (deletions and amino acid substitutions) shown in FIG. 3.

SEQ ID NO: 8 Flu080 stem only construct—B/Washington/02/2019 sequence, details as for SEQ ID NO: 2, with specific mutations (deletions and amino acid substitutions) shown in FIG. 3.

SEQ ID NO: 9 Flu081 full ectodomain construct—B/Washington/02/2019 sequence with mutations shown in FIG. 4; linker found between ectodomain and transmembrane sequence in wildtype removed (see sequence alignment in FIG. 2(b)); added foldon and poly histidine tail.

SEQ ID NO: 10 Flu082 full ectodomain construct—B/Washington/02/2019 sequence, details as for SEQ ID NO: 9.

SEQ ID NO: 11 Flu083 full ectodomain construct—B/Washington/02/2019 sequence, details as for SEQ ID NO: 9.

SEQ ID NO: 12 Flu084 full ectodomain construct—B/Washington/02/2019 sequence, details as for SEQ ID NO: 9.

SEQ ID NO: 13 Flu087 full ectodomain construct—B/Washington/02/2019 sequence, details as for SEQ ID NO: 9.

SEQ ID NO: 14 Flu090 full ectodomain construct—B/Washington/02/2019 sequence, details as for SEQ ID NO: 9.

SEQ ID NO: 15 Flu111 full ectodomain construct—B/Washington/02/2019 sequence, details as for SEQ ID NO: 9.

SEQ ID NO: 16 Flu112 full ectodomain construct—B/Washington/02/2019 sequence, details as for

SEQ ID NO: 9.

SEQ ID NO: 17 Flu115 full ectodomain construct—B/Washington/02/2019 sequence, details as for SEQ ID NO: 9.

SEQ ID NO: 18 Flu119 full ectodomain construct—B/Washington/02/2019 sequence, details as for SEQ ID NO: 9.

SEQ ID NO: 19 Flu136 full ectodomain construct—B/Washington/02/2019 sequence, details as for SEQ ID NO: 9.

SEQ ID NO: 20 Flu159 full ectodomain construct—B/Washington/02/2019 sequence, details as for SEQ ID NO: 9.

SEQ ID NO: 21 Flu161 full ectodomain construct—B/Washington/02/2019 sequence, details as for SEQ ID NO: 9.

SEQ ID NO: 22 Flu182 full ectodomain construct—B/Washington/02/2019 sequence, details as for SEQ ID NO: 9.

SEQ ID NO: 23 Flu187 full ectodomain construct—B/Washington/02/2019 sequence, details as for SEQ ID NO: 9.

SEQ ID NO: 24 Flu188 full ectodomain construct—B/Washington/02/2019 sequence, details as for SEQ ID NO: 9.

SEQ ID NO: 25 Flu203 full ectodomain construct—B/Washington/02/2019 sequence, details as for SEQ ID NO: 9.

SEQ ID NO: 26 Foldon sequence

SEQ ID NO: 27 Signal peptide from B Victoria strain B/Washington/02/2019

SEQ ID NO: 28 Transmembrane domain from influenza B/Washington/02/2019 (Victoria)

SEQ ID NO: 29 Coding sequence for Flu018

SEQ ID NO: 30 Coding sequence for Flu029

SEQ ID NO: 31 Coding sequence for Flu035

SEQ ID NO: 32 Coding sequence for Flu036

SEQ ID NO: 33 Coding sequence for Flu075

SEQ ID NO: 34 Coding sequence for Flu077

SEQ ID NO: 35 Coding sequence for Flu080

SEQ ID NO: 36 Coding sequence for Flu081

SEQ ID NO: 37 Coding sequence for Flu082

SEQ ID NO: 38 Coding sequence for Flu083

SEQ ID NO: 39 Coding sequence for Flu084

SEQ ID NO: 40 Coding sequence for Flu087

SEQ ID NO: 41 Coding sequence for Flu090

SEQ ID NO: 42 Coding sequence for Flu111

SEQ ID NO: 43 Coding sequence for Flu112

SEQ ID NO: 44 Coding sequence for Flu115

SEQ ID NO: 45 Coding sequence for Flu119

SEQ ID NO: 46 Coding sequence for Flu136

SEQ ID NO: 47 Coding sequence for Flu159

SEQ ID NO: 48 Coding sequence for Flu161

SEQ ID NO: 49 Coding sequence for Flu182

SEQ ID NO: 50 Coding sequence for Flu187

SEQ ID NO: 51 Coding sequence for Flu188

SEQ ID NO: 52 Coding sequence for Flu203

SEQ ID NO: 53 4 amino acid linker sequence

SEQ ID NO: 54 4 amino acid linker sequence

SEQ ID NO: 55 4 amino acid linker sequence

SEQ ID NO: 56 5 amino acid linker sequence

SEQ ID NO: 57 5 amino acid linker sequence

SEQ ID NO: 58 6 amino acid linker sequence

SEQ ID NOs: 59-62 amino acid linker sequences used in stem only constructs of batch 2, shown in FIGS. 3(b) and 5(d)

SEQ ID NOs: 63-82 amino acid linker sequences used in stem only constructs of batch 3, shown in FIG. 3(c)

SEQ ID NO: 83: HA sequence from B Victoria strain B/Washington/02/2019 (Wash19) ectodomain from FIG. 8, showing Regions A, B and C of the fusion domain region

SEQ ID NO: 84 Flu209 full ectodomain construct—B/Washington/02/2019 sequence with mutations shown in FIG. 4; linker found between ectodomain and transmembrane sequence in wildtype removed (see sequence alignment in FIG. 2(c)); added foldon and poly histidine tail.

SEQ ID NO: 85 Flu212 full ectodomain construct—B/Washington/02/2019 sequence, details as for SEQ ID NO: 84.

SEQ ID NO: 86 Flu227 full ectodomain construct—B/Washington/02/2019 sequence, details as for SEQ ID NO: 84.

SEQ ID NO: 87 Flu245 full ectodomain construct—B/Washington/02/2019 sequence, details as for SEQ ID NO: 84.

SEQ ID NO: 88 Flu250 full ectodomain construct—B/Washington/02/2019 sequence, details as for SEQ ID NO: 84.

SEQ ID NO: 89 Flu251 full ectodomain construct—B/Washington/02/2019 sequence, details as for SEQ ID NO: 84.

SEQ ID NO: 90 Flu254 full ectodomain construct—B/Washington/02/2019 sequence, details as for SEQ ID NO: 84.

SEQ ID NO: 91 Flu256 full ectodomain construct—B/Washington/02/2019 sequence, details as for SEQ ID NO: 84.

SEQ ID NO: 92 Flu260 full ectodomain construct—B/Washington/02/2019 sequence, details as for SEQ ID NO: 84.

SEQ ID NO: 93 Flu262 full ectodomain construct—B/Washington/02/2019 sequence, details as for SEQ ID NO: 84.

SEQ ID NO: 94 Flu264 full ectodomain construct—B/Washington/02/2019 sequence, details as for SEQ ID NO: 84.

SEQ ID NO: 95 Flu266 full ectodomain construct—B/Washington/02/2019 sequence, details as for SEQ ID NO: 84.

SEQ ID NO: 96 Flu289 stem only construct—B/Washington/02/2019 HA sequence with mutations (deletions and amino acid substitution in this case) and added linkers shown in FIG. 3; linker found between ectodomain and transmembrane sequence in wildtype removed (see sequence alignment in FIG. 2(c)); added foldon and poly histidine tail.

SEQ ID NO: 97 Flu295 stem only construct—B/Washington/02/2019 sequence as for SEQ ID NO: 96, with specific mutations (deletions and amino acid substitutions) shown in FIG. 3.

SEQ ID NO: 98 Flu303 stem only construct—B/Washington/02/2019 sequence as for SEQ ID NO: 96, with specific mutations (deletions and amino acid substitutions) shown in FIG. 3.

SEQ ID NO: 99 Flu309 stem only construct—B/Washington/02/2019 sequence as for SEQ ID NO: 96, with specific mutations (deletions and amino acid substitutions) shown in FIG. 3.

SEQ ID NO: 100 Flu330 stem only construct—B/Washington/02/2019 sequence as for SEQ ID NO: 96, with specific mutations (deletions and amino acid substitutions) shown in FIG. 3.

SEQ ID NO: 101 Flu333 stem only construct—B/Washington/02/2019 sequence as for SEQ ID NO: 96, with specific mutations (deletions and amino acid substitutions) shown in FIG. 3.

SEQ ID NO: 102 Flu337 stem only construct—B/Washington/02/2019 sequence as for SEQ ID NO: 96, with specific mutations (deletions and amino acid substitutions) shown in FIG. 3.

SEQ ID NO: 103 Flu378 stem only construct—B/Washington/02/2019 sequence as for SEQ ID NO: 96, with specific mutations (deletions and amino acid substitutions) shown in FIG. 3.

SEQ ID NO: 104 Flu389 stem only construct—B/Washington/02/2019 sequence as for SEQ ID NO: 96, with specific mutations (deletions and amino acid substitutions) shown in FIG. 3.

SEQ ID NO: 105 Flu392 stem only construct—B/Washington/02/2019 sequence as for SEQ ID NO: 96, with specific mutations (deletions and amino acid substitutions) shown in FIG. 3.

SEQ ID NO: 106 Flu408 stem only construct—B/Washington/02/2019 sequence as for SEQ ID NO: 96, with specific mutations (deletions) shown in FIG. 3.

SEQ ID NO: 107 Flu412 stem only construct—B/Washington/02/2019 sequence as for SEQ ID NO: 96, with specific mutations (deletions) shown in FIG. 3.

SEQ ID NO: 108 Flu452 stem only construct—based on Flu337 backbone, with mutations and linkers shown in FIG. 3(c).

SEQ ID NO: 109 Flu460 stem only construct—based on Flu337 backbone, with mutations and linkers shown in FIG. 3(c).

SEQ ID NO: 110 Flu468 stem only construct—based on Flu337 backbone, with mutations and linkers shown in FIG. 3(c).

SEQ ID NO: 111 Flu474 stem only construct—based on Flu337 backbone, with mutations and linkers shown in FIG. 3(c).

SEQ ID NO: 112 Flu482 stem only construct—based on Flu337 backbone, with mutations and linkers shown in FIG. 3(c).

SEQ ID NO: 113 Flu490 stem only construct—based on Flu337 backbone, with mutations and linkers shown in FIG. 3(c).

SEQ ID NO: 114 Flu496 stem only construct—based on Flu337 backbone, with mutations and linkers shown in FIG. 3(c).

SEQ ID NO: 115 Flu508 stem only construct—based on Flu337 backbone, with mutations and linkers shown in FIG. 3(c).

SEQ ID NO: 116 Flu512 stem only construct—based on Flu309 backbone, with mutations and linkers shown in FIG. 3(c).

SEQ ID NO: 117 Flu518 stem only construct—based on Flu309 backbone, with mutations and linkers shown in FIG. 3(c).

SEQ ID NO: 118 Flu523 stem only construct—based on Flu309 backbone, with mutations and linkers shown in FIG. 3(c).

SEQ ID NO: 119 Flu526 stem only construct—based on Flu309 backbone, with mutations and linkers shown in FIG. 3(c).

SEQ ID NO: 120 Flu534 stem only construct—based on Flu309 backbone, with mutations and linkers shown in FIG. 3(c).

SEQ ID NO: 121 Flu536 stem only construct—based on Flu309 backbone, with mutations and linkers shown in FIG. 3(c).

SEQ ID NO: 122 coding sequence for Flu209 SEQ ID NO: 123 coding sequence for Flu212

SEQ ID NO: 124 coding sequence for Flu227

SEQ ID NO: 125 coding sequence for Flu245

SEQ ID NO: 126 coding sequence for Flu250

SEQ ID NO: 127 coding sequence for Flu251

SEQ ID NO: 128 coding sequence for Flu254

SEQ ID NO: 129 coding sequence for Flu256

SEQ ID NO: 130 coding sequence for Flu260

SEQ ID NO: 131 coding sequence for Flu262

SEQ ID NO: 132 coding sequence for Flu264

SEQ ID NO: 133 coding sequence for Flu266

SEQ ID NO: 134 coding sequence for Flu289

SEQ ID NO: 135 coding sequence for Flu295

SEQ ID NO: 136 coding sequence for Flu303

SEQ ID NO: 137 coding sequence for Flu309

SEQ ID NO: 138 coding sequence for Flu330

SEQ ID NO: 139 coding sequence for Flu333

SEQ ID NO: 140 coding sequence for Flu337

SEQ ID NO: 141 coding sequence for Flu378

SEQ ID NO: 142 coding sequence for Flu389

SEQ ID NO: 143 coding sequence for Flu392

SEQ ID NO: 144 coding sequence for Flu408

SEQ ID NO: 145 coding sequence for Flu412

SEQ ID NO: 146 coding sequence for Flu452

SEQ ID NO: 147 coding sequence for Flu460

SEQ ID NO: 148 coding sequence for Flu468

SEQ ID NO: 149 coding sequence for Flu474

SEQ ID NO: 150 coding sequence for Flu482

SEQ ID NO: 151 coding sequence for Flu490

SEQ ID NO: 152 coding sequence for Flu496

SEQ ID NO: 153 coding sequence for Flu508

SEQ ID NO: 154 coding sequence for Flu512

SEQ ID NO: 155 coding sequence for Flu518

SEQ ID NO: 156 coding sequence for Flu523

SEQ ID NO: 157 coding sequence for Flu526

SEQ ID NO: 158 coding sequence for Flu534

SEQ ID NO: 159 coding sequence for Flu536

SEQ ID NO: 160 Flu 075 stem only construct fused to membrane anchoring region

SEQ ID NO: 161 Flu 075 stem only construct fused to H. pylori ferritin

SEQ ID NO: 162 Flu 077 stem only construct fused to membrane anchoring region

SEQ ID NO: 163 Flu 077 stem only construct fused to H. pylori ferritin

SEQ ID NO: 164 Flu 115 full ectodomain construct fused to membrane anchoring region

SEQ ID NO: 165 Flu 115 full ectodomain construct fused to H. pylori ferritin

SEQ ID NO: 166 Flu 159 full ectodomain construct fused to membrane anchoring region

SEQ ID NO: 167 Flu 159 full ectodomain construct fused to H. pylori ferritin

SEQ ID NO: 168 Flu 460 stem only construct fused to membrane anchoring region

SEQ ID NO: 169 Flu 460 stem only construct fused to H. pylori ferritin

SEQ ID NO: 170 Flu 474 stem only construct fused to membrane anchoring region

SEQ ID NO: 171 Flu 474 stem only construct fused to H. pylori ferritin

SEQ ID NO: 172 Flu 490 stem only construct fused to membrane anchoring region

SEQ ID NO: 173 Flu 490 stem only construct fused to H. pylori ferritin

SEQ ID NO: 174 Flu 523 stem only construct fused to membrane anchoring region

SEQ ID NO: 175 Flu 523 stem only construct fused to H. pylori ferritin

SEQ ID NO: 176 Flu 209 full ectodomain construct fused to membrane anchoring region

SEQ ID NO: 177 Flu 209 full ectodomain construct fused to H. pylori ferritin

SEQ ID NO: 178 Flu 256 full ectodomain construct fused to membrane anchoring region

SEQ ID NO: 179 Flu 256 full ectodomain construct fused to H. pylori ferritin

### BRIEF DESCRIPTION OF THE FIGURES

FIG. 1: Influenza B strain haemagglutinin conservation analysis: (a) N terminal stem region of HA1; (b) C terminal stem region of HA1; and (c) HA2.

FIG. 2: (a) B/Washington/02/2019 (Victoria) Haemagglutinin sequence-annotated;

(b) Sequence alignment for B strain HA, top line showing full length sequence of the wild type HA polypeptide B/Washington/02/2019 (Wash19), including transmembrane and cytoplasmic domain, and below that Flu018 to Flu080 (HA stem only constructs) and Flu081 to Flu203 (full HA ectodomain constructs) all based on B Victoria strain B/Washington/02/2019 (Wash19).

(c) Sequence alignment as for (b), showing Flu209 to Flu266 (full HA ectodomain constructs) and Flu289 to Flu412 (HA stem only constructs) all based on B Victoria strain B/Washington/02/2019 (Wash19).

(d) Sequence alignment as for (b) and (c), showing Flu452 to Flu508 (stem only constructs) all based on B Victoria strain B/Washington/02/2019 (Wash19), based on a Flu337 backbone.

(e) Sequence alignment as for (c), showing Flu512 to Flu536 (stem only constructs) based on a Flu309 backbone.

FIG. 3: Table showing mutations for selected stem only constructs from (a) batch 1 Flu018 to Flu080, (b) batch 2 Flu289 to Flu412 and (c) batch 3 Flu452 to Flu536.

FIG. 4: Table showing amino acid substitutions for selected full HA ectodomain constructs from (a) batch 1 Flu081 to Flu203 and (b) batch 2 Flu209 to Flu226.

FIG. 5: Table showing constructs Flu001 to Flu415: (a) and (d) stem constructs with deletions, substitutions and linkers; and (b) and (c) ectodomain constructs with mutations.

FIG. 6: Diagram showing 3D model of influenza B strain HA timer in prefusion form and HA monomer.

FIG. 7: Diagrams showing 3D model of influenza B strain haemagglutinin full ectodomain and stem constructs with location of CR9114 and 5A7 epitopes.

FIG. 8: Sequence and 3D model of influenza B strain haemagglutinin ectodomain showing Regions A, B and C of the fusion domain region.

FIG. 9: Schematic diagram showing influenza B strain haemagglutinin ectodomain and stem constructs from batch 1 (Flu001-Flu207).

FIG. 10: Protein purification analysis carried out by SDS-PAGE: a) proteins corresponding to different truncated HA ectodomain mutants and b) different HA ectodomain mutants.

FIG. 11: Antibody binding analysis. Biolayer interferometry (BLI) results for full ectodomain (a and b) and stem (c and d).

FIG. 12: Protein characterization of full ectodomain (a and b) and stem (c) by nano differential scanning fluorimetry (NanoDSF).

FIG. 13: Protein characterisation of full HA ectodomain (a and b) and stem (c) by sedimentation velocity analytical ultracentrifugation (SV-AUC), before and after trimerization domain removal.

FIG. 14: Anti-B/Washington/2/2019 (full HA, B/Victoria) IgG antibodies by ELISA at 14 days post dose 2 following immunization of mice with full ectodomain and stem only constructs.

FIG. 15: Anti-B/Illinois/NHRC_18512/2017 (full HA, B/Victoria) IgG antibodies by ELISA at 14 days post dose 2 following immunization of mice with full ectodomain and stem only constructs.

FIG. 16: Anti-B/Phuket/3073/2013 (full HA, B/Yamagata) IgG antibodies by ELISA at 14 days post dose 2 following immunization of mice with full ectodomain and stem only constructs.

FIG. 17: Anti-B/Washington/2/2019 (stem only HA, B/Victoria) IgG antibodies by ELISA at 14 days post dose 2 following immunization of mice with full ectodomain and stem only constructs.

FIG. 18: Anti-B/Washington/2/2019 (full HA, B/Victoria) IgG antibodies by ELISA at 14 days post dose 2 following immunization of mice with stem only constructs with and without ferritin.

FIG. 19: Anti-Anti-B/Illinois/NHRC_18512/2017 (full HA, B/Victoria) IgG antibodies by ELISA at 14 days post dose 2 following immunization of mice with stem only constructs with and without ferritin.

FIG. 20: Anti-B/Phuket/3073/2013 (full HA, B/Yamagata) IgG antibodies by ELISA at 14 days post dose 2 following immunization of mice with stem only constructs with and without ferritin.

FIG. 21: Anti-B/Washington/2/2019 (B/Victoria) HI antibodies at 14 days post dose 2 following immunization of mice with full ectodomain constructs.

FIG. 22: Anti-B/Darwin/7/2019 (B/Victoria) HI antibodies at 14 days post dose 2 following immunization of mice with full ectodomain constructs.

FIG. 23: Anti-B/Austria/1359417/2021 (B/Victoria) HI antibodies at 14 days post dose 2 following immunization of mice with full ectodomain constructs.

FIG. 24: Anti-B/Maryland/15/2016 (B/Victoria) HI antibodies at 14 days post dose 2 following immunization of mice with full ectodomain constructs.

FIG. 25: Anti-B/Phuket/3073/2013 (B/Yamagata) HI antibodies at 14 days post dose 2 following immunization of mice with full ectodomain constructs.

FIG. 26: Anti-B/Massachusetts/02/2012 (B/Yamagata) HI antibodies at 14 days post dose 2 following immunization of mice with full ectodomain constructs.

FIG. 27: Anti-B/Washington/2/2019 stem antibodies by ADCC Reporter Bioassay at 14 days post dose 2 following immunization of mice with full ectodomain and stem only constructs

### DETAILED DESCRIPTION

### HA Stem and Ectodomain

The recombinant influenza B strain HA antigen described herein contains mutations such as amino acid deletions, substitutions (e.g. point mutations) or additions compared to the wild type HA amino acid sequence.

The influenza B strain HA stem domain is located in the membrane-proximal region of the native HA, directly beneath the vestigial esterase domain of the HA1 globular head (see FIG. 7). The native influenza HA stem is comprised of amino acid residues from both ends of the HA1 chain as well as the ectodomain part of the HA2 chain. A section of around 250-300 amino acid residues in the centre of the HA1 sequence forms the HA globular head and does not form a part of the HA stem domain.

The HA head domain is the globular head region of the HA protein, excluding the stem, the transmembrane domain and any intracellular region. The HA head is composed of part of HA1 and contains a sialic acid binding pocket that mediates virus attachment to the host cell. The HA head is comprised of the receptor binding domain and the vestigial esterase domain (see FIG. 7).

In one embodiment, the recombinant influenza B strain HA antigen comprises an HA stem domain in the absence of an HA head domain. Influenza B strain HA stem antigens may be obtained by deleting certain sections of the HA sequence that form the head domain, resulting in a stem antigen without a globular head domain. In particular embodiments, one or more deletions compared to native HA, that result in an HA stem antigen without an HA head domain, are located in HA1, typically a deletion of 240-300 or 250-300 contiguous amino acids from HA1. There may also be one or more deletions located in HA2 compared to native HA2, typically including a shorter deletion of 5 or more, or 10 or more, or 10-40 or 20-40 or 25-30 contiguous amino acids from HA2. In one embodiment, a section deleted from HA2 forms part of the coiled coil structure in native HA that is not normally exposed to the immune system as it is buried inside the HA trimer. Thus in one embodiment, the HA stem antigen has both a stretch of contiguous amino acids deleted from HA1 as well as a stretch of contiguous amino acids deleted from HA2, for example 240-300 or 250-300 contiguous amino acids deleted from HA1 and a shorter deletion of for example 5 or more, or 10 or more e.g. 10-40 or 20-40 or 25-30 contiguous amino acids deleted from HA2, with either or both deleted sequences optionally replaced by a suitable linker. Examples of suitable linkers are G, GS, GSG and linkers shown in SEQ ID NOs: 53-82 and as discussed hereinbelow. In one embodiment, the HA1 deletion and/or the HA2 deletions are from within HA1 and/or HA2 and not at an end of HA1 and/or HA2, that is the deletion is not at or does not include an amino acid at the N or the C terminus of HA1 and/or HA2 (where the N terminus of HA1 is considered to be after the signal sequence). The HA stem antigen may additionally contain one or more amino acid substitutions designed to stabilise the antigen, as described herein.

In one embodiment, the recombinant influenza B strain HA stem antigen comprises HA1 with a central region of the HA1 deleted, for example a deletion of between 200 and 300 amino acids, or at least 200, or at least 250, or around 245 amino acids, or around 293, or around 295 contiguous amino acids from HA1. The deleted amino acids form a part of the head domain in native HA. In a particular embodiment, the recombinant HA stem further comprises HA2 with a central region of the HA2 deleted, for example a deletion of between 15 and 40 amino acids, or at least 20 or at least 30 or around 23 or around 31 or around 33 or around 36 or around 38 or around 39 contiguous amino acids from HA2. By ‘central region’ of HA1 or HA2 is meant not including the end portions of the HA1 or the HA2, for example not including the 5 or 10 or 15 or 20 or 25 amino acids at the N and the C terminus of the HA1 or HA2 (where the N terminus of HA1 does not include the signal sequence). In one embodiment, the recombinant HA stem antigen comprises deletions in both HA1 and HA2, for example a deletion of between 200 and 300 amino acids from HA1 and between 15 and 40 amino acids from HA2.

A deletion of contiguous amino acids from HA1 compared to wild type may be referred to herein as deletion 1. A deletion of contiguous amino acids from HA2 compared to wild type may be referred to herein as deletion 2.

In a particular embodiment the stem antigen has a deletion 1 from a position between amino acids 47-58 to a position between amino acids 302-341, such as a deletion selected from L47 to L341, T48 to P340, or N58 to 1302.

In a particular embodiment the stem antigen has a deletion 2 from a position between amino acids 413-422 to a position between amino acids 444-452, such as a deletion selected from S413 to 1451, S413 to A448, V419 to 1451, S415 to D445, S415 to S452 or L422 to D444.

In particular embodiments, the influenza B recombinant HA stem antigen has sequences deleted from HA1 and HA2, compared to the wild type HA, selected from one of the following combinations:
N58-1302 (HA1) and L422-D444 (HA2) (Group 2)
T48-P340 (HA1) and L422-D444 (HA2) (Group 3)
T48-P340 (HA1) and V419-1451 (HS2) (Group 4)
T48-P340 (HA1) and S413-A448 (HA2) (Group 3)
T48-P340 (HA1) and S413-1451 (HA2) (Group 4)
L47-L341 (HA1) and S415-S452 (HA2) (Group 4)
L47-L341 (HA1) and S415-D445 (HA2) (Group 3)

Groups 1 to 4 describe the extent of the truncation of the HA, where Group 4 represents the most highly truncated backbone and Group 1 the least truncated (Group 1 not shown).

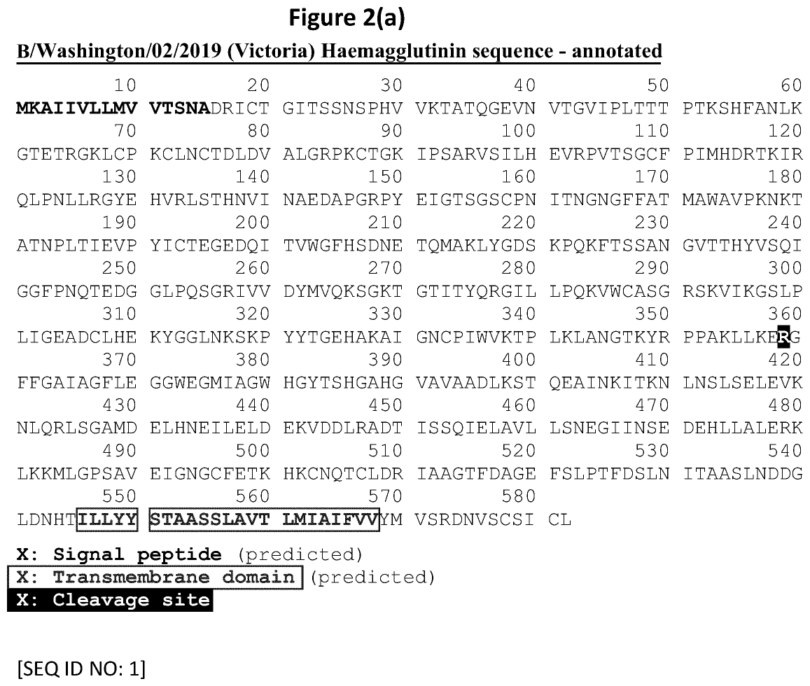

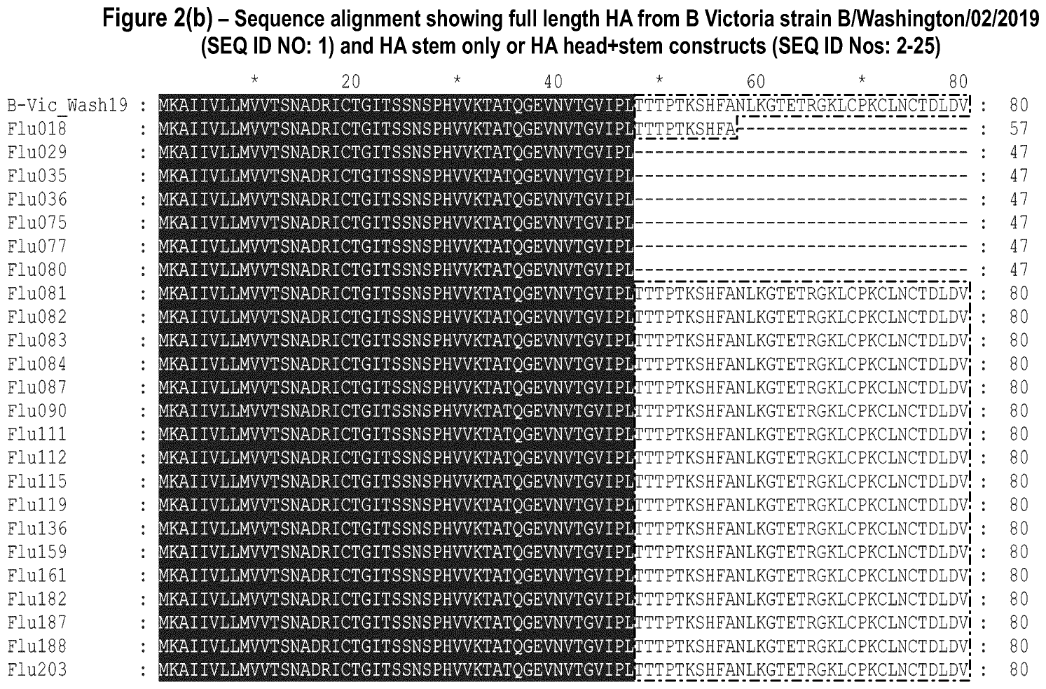

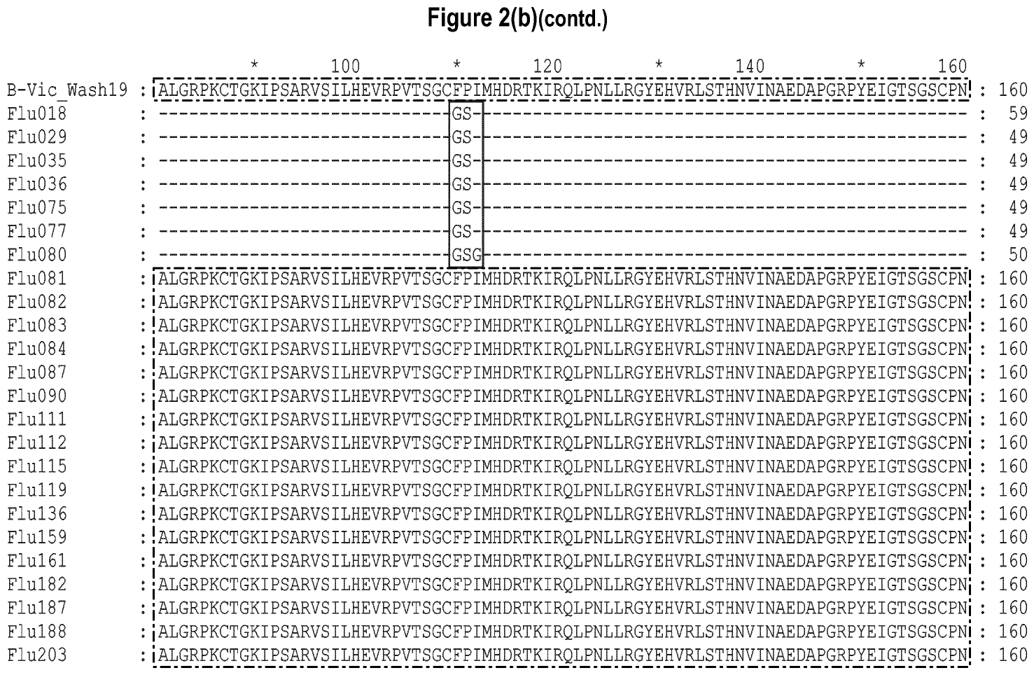

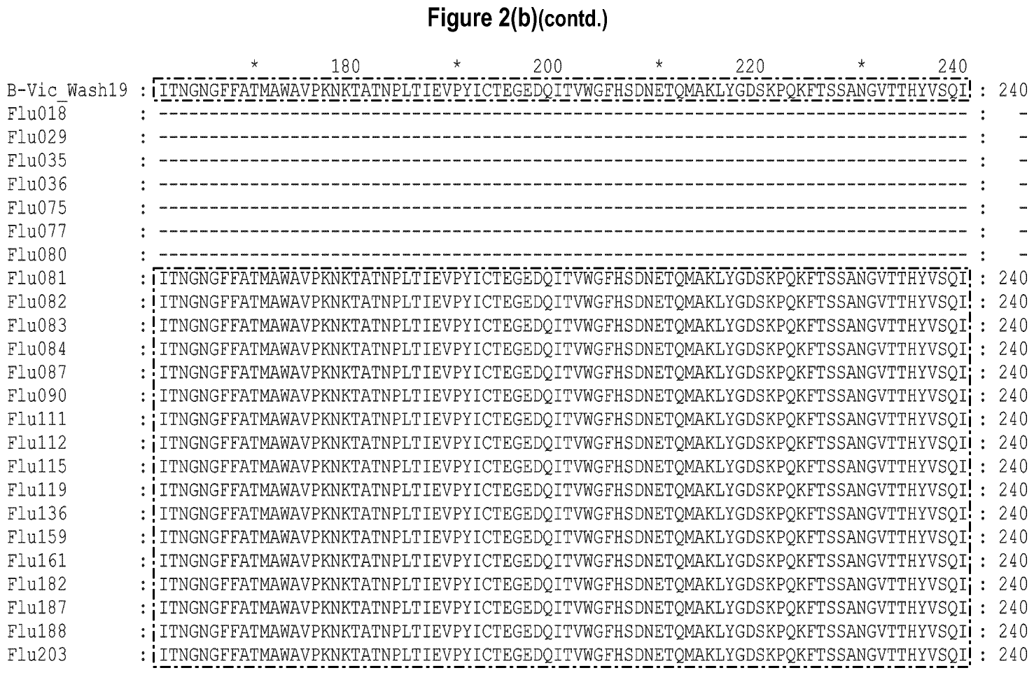

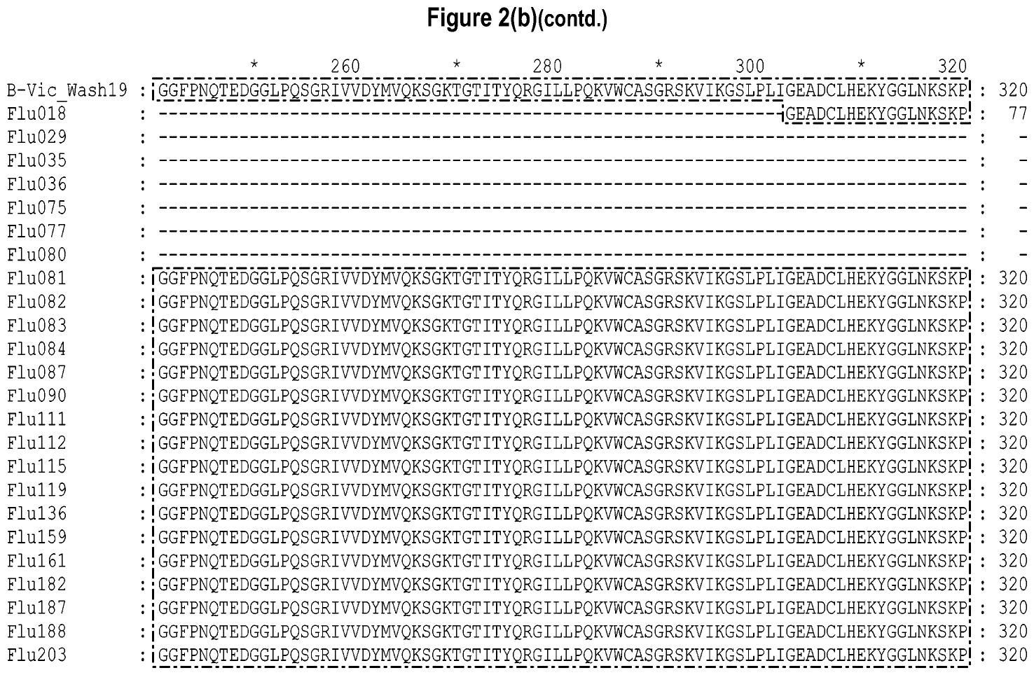

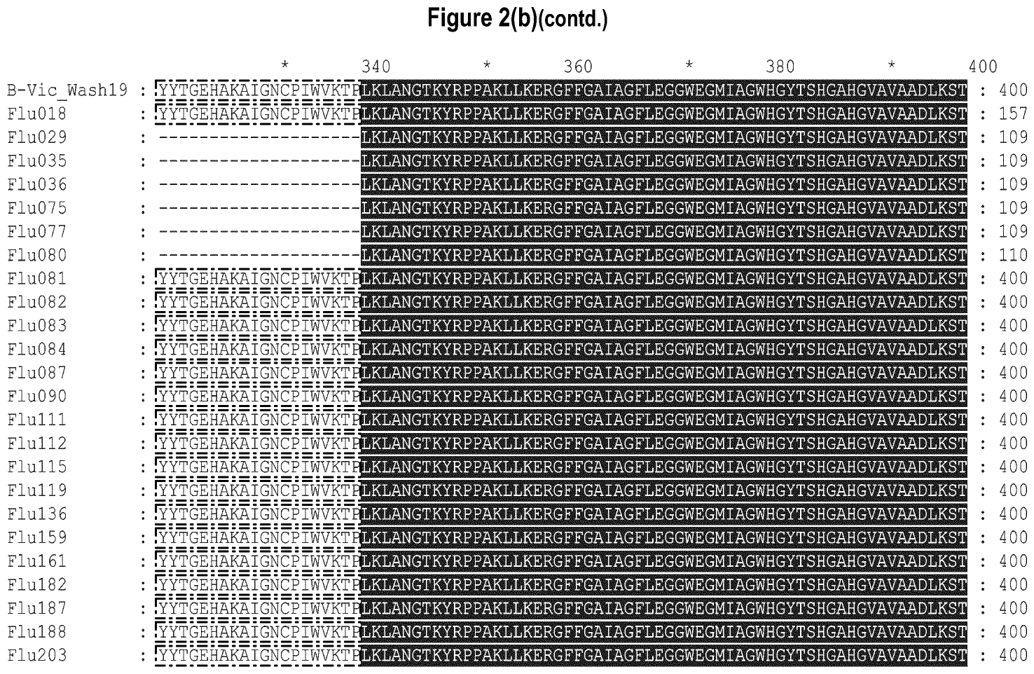

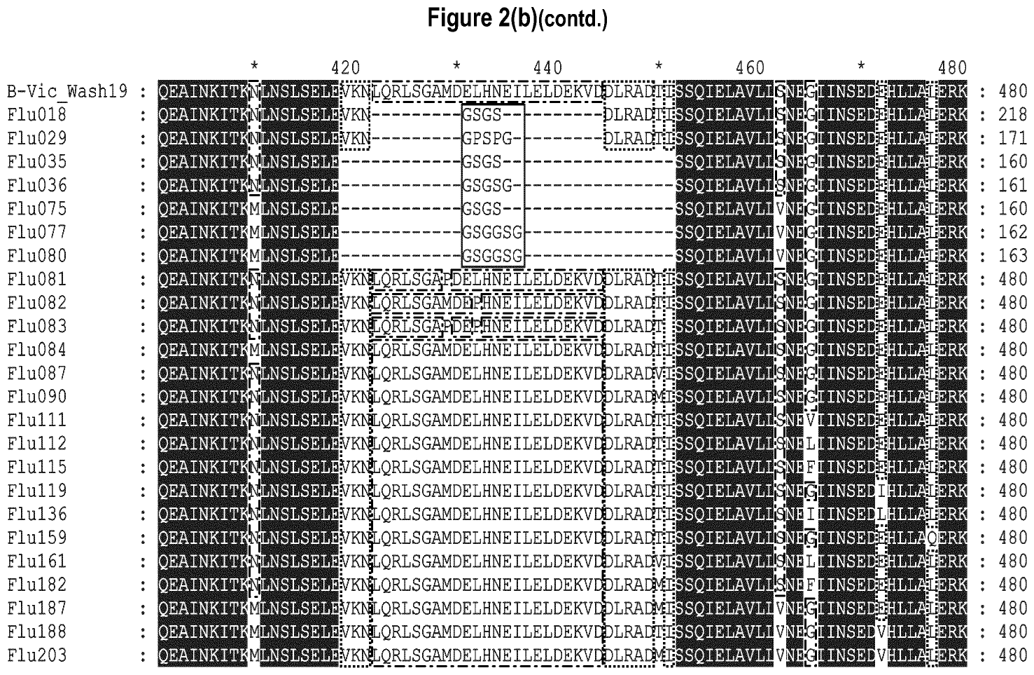

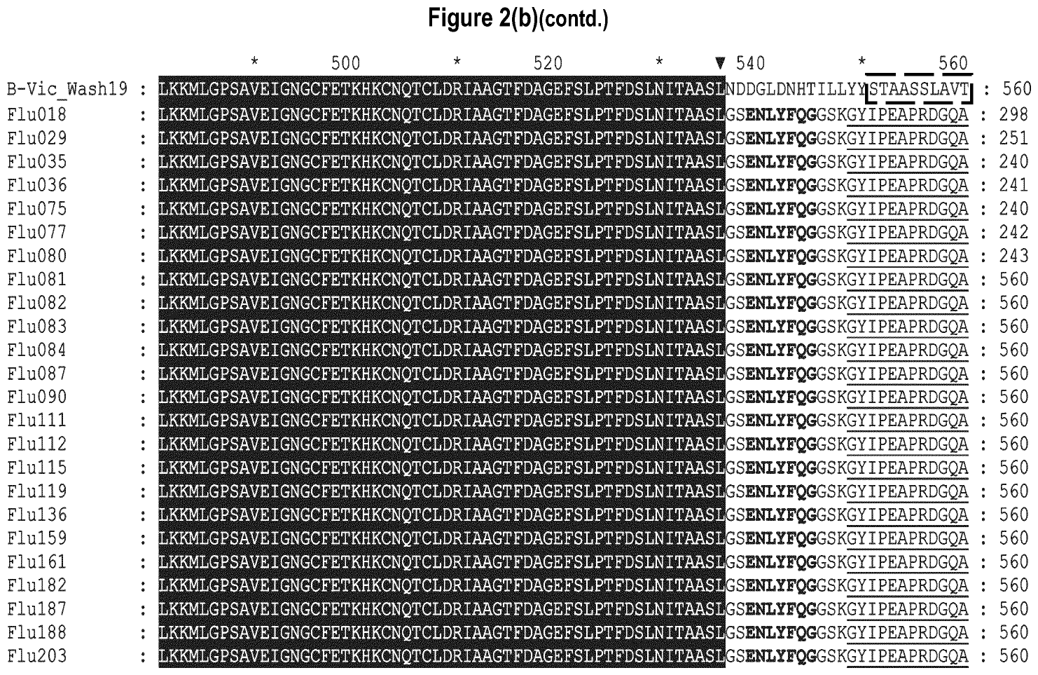

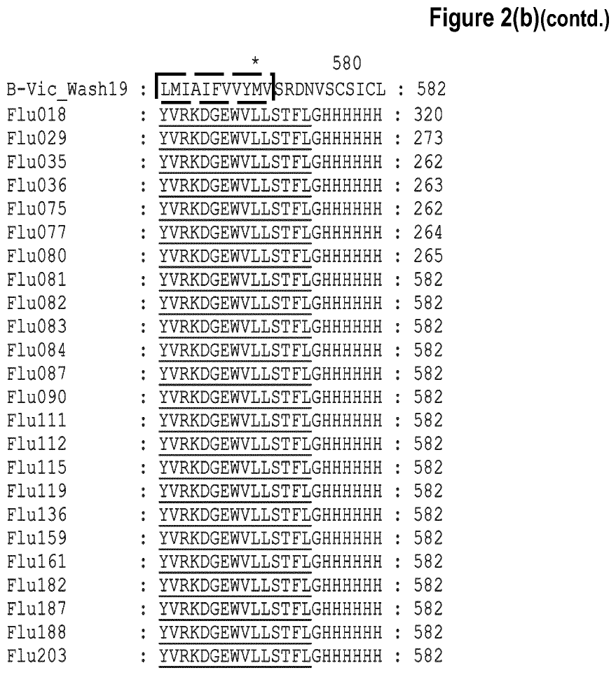

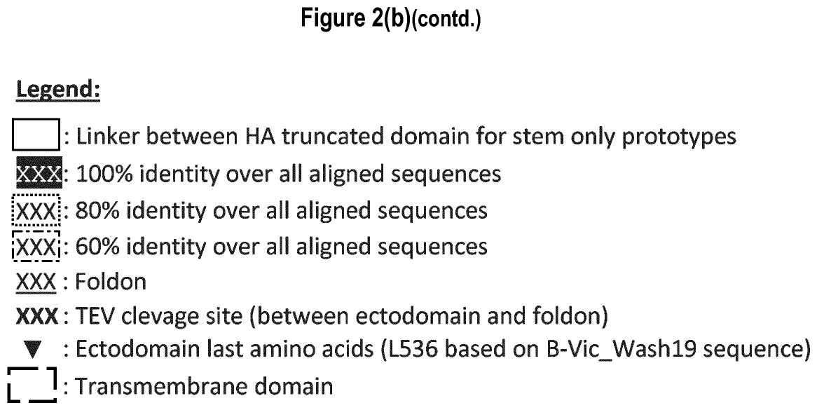

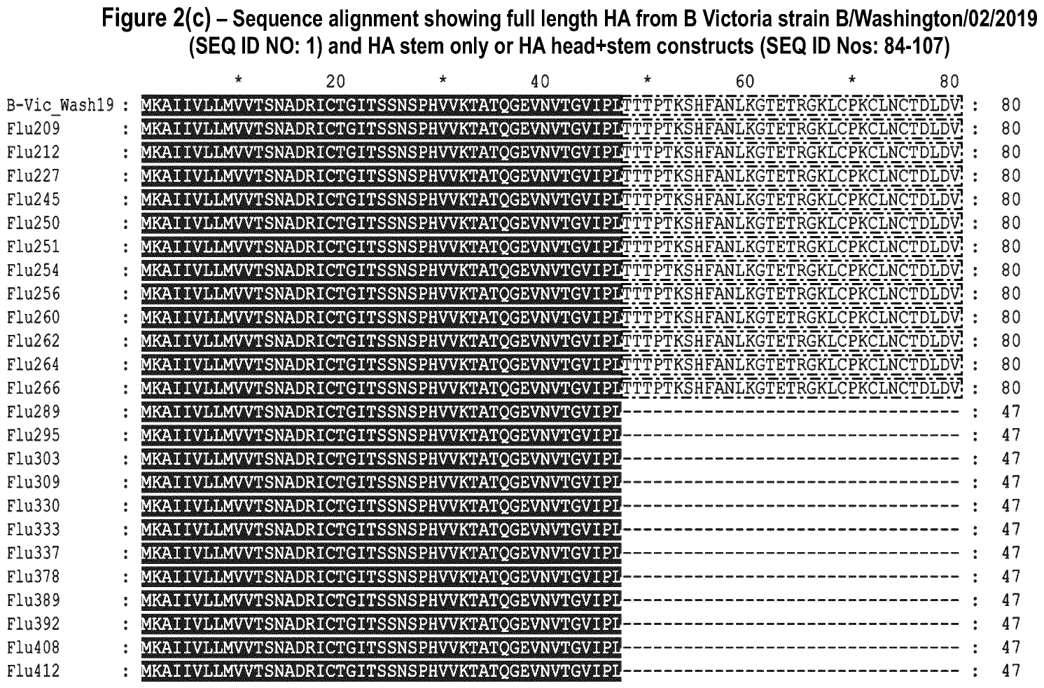

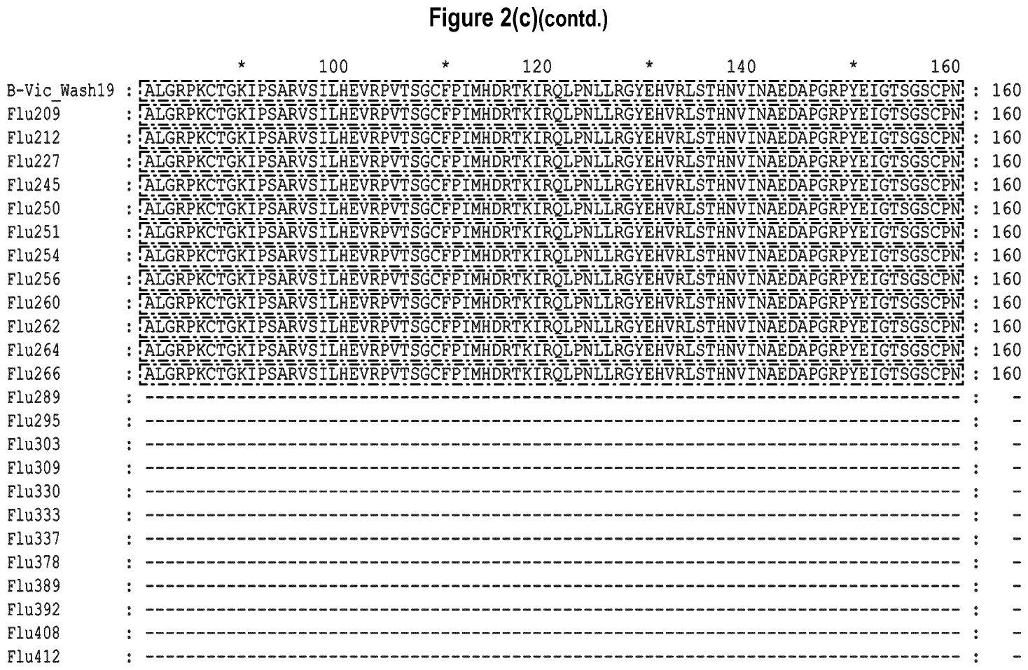

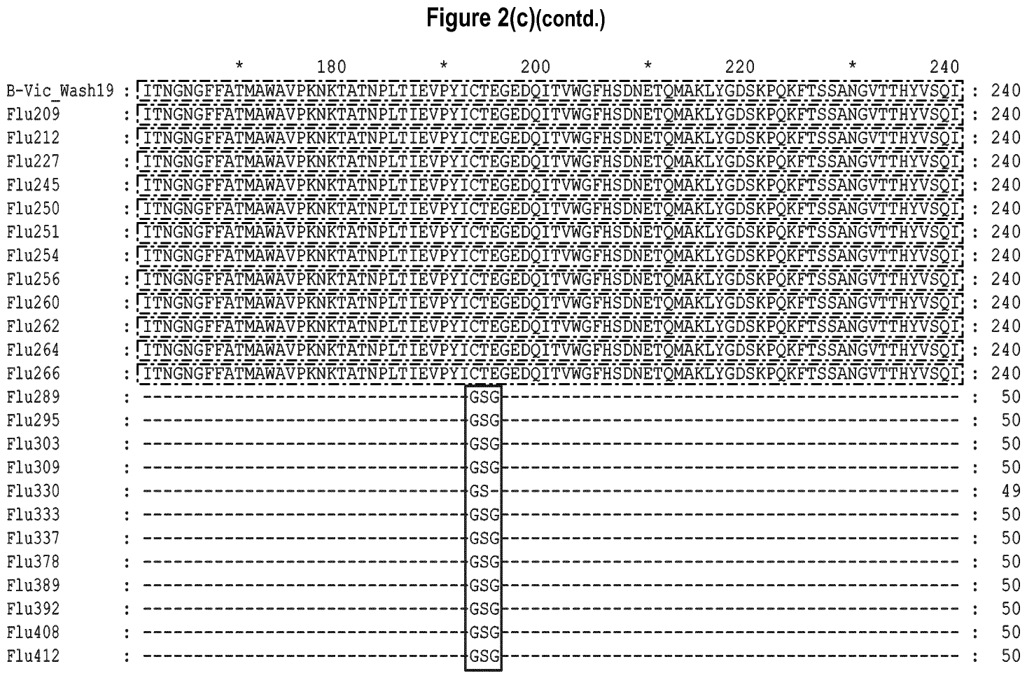

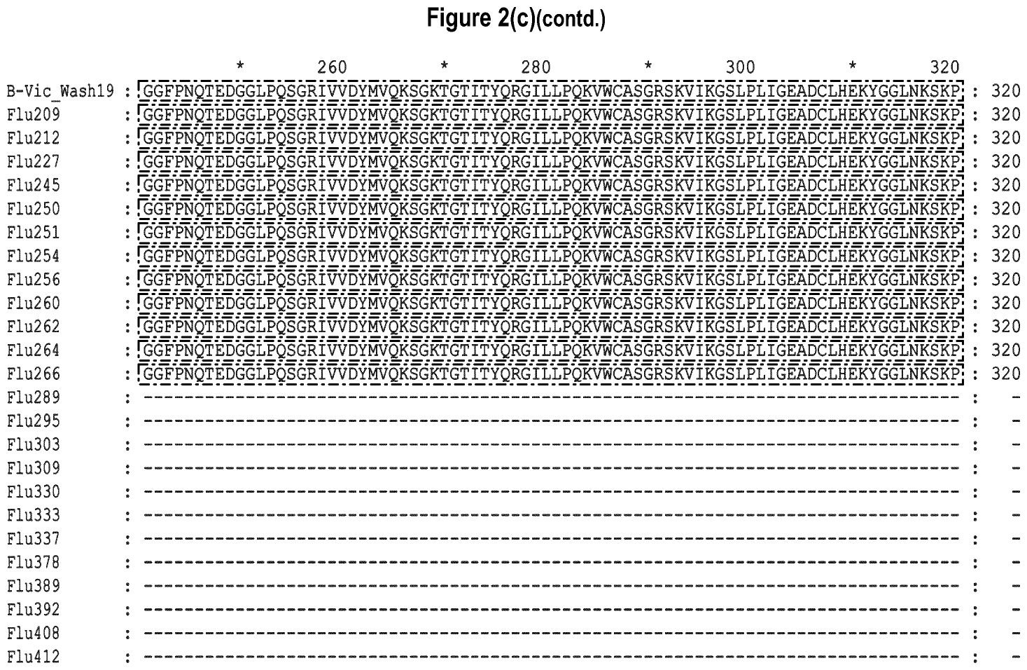

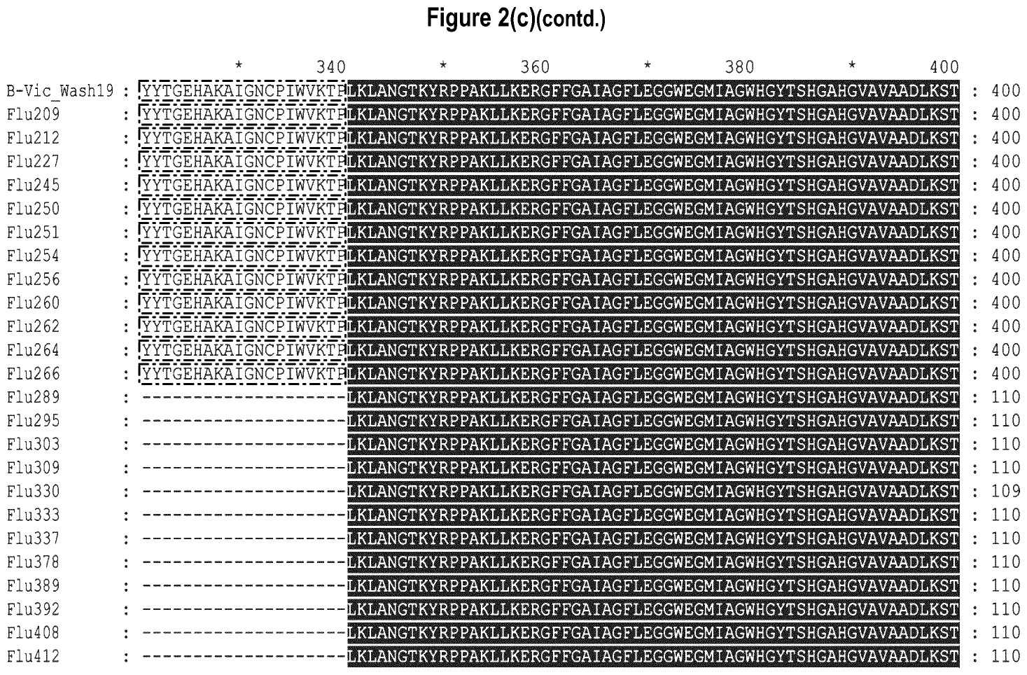

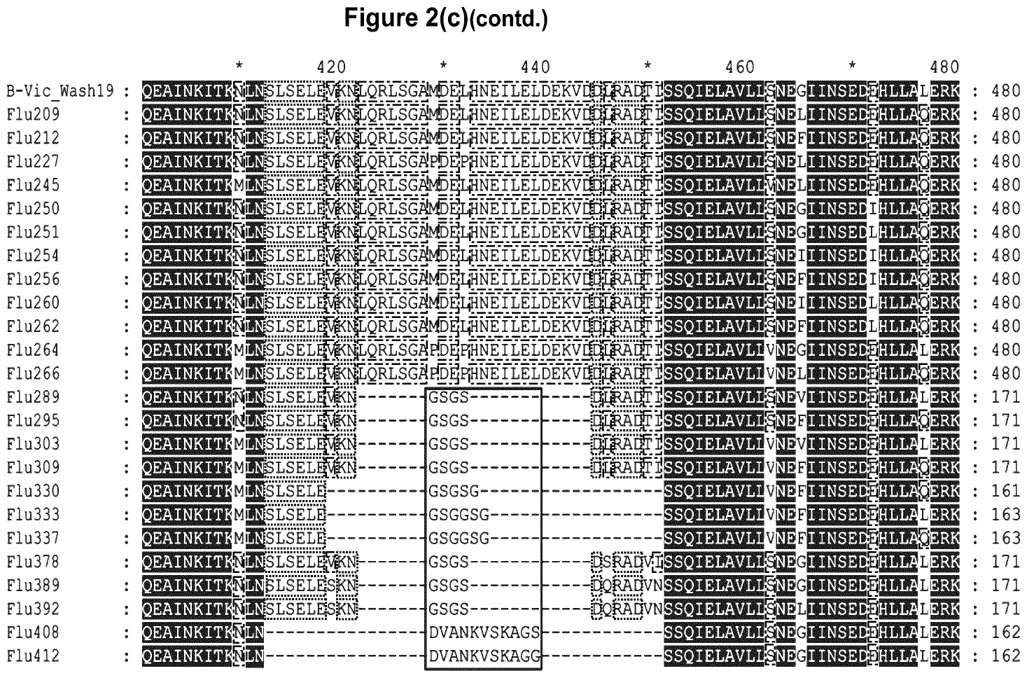

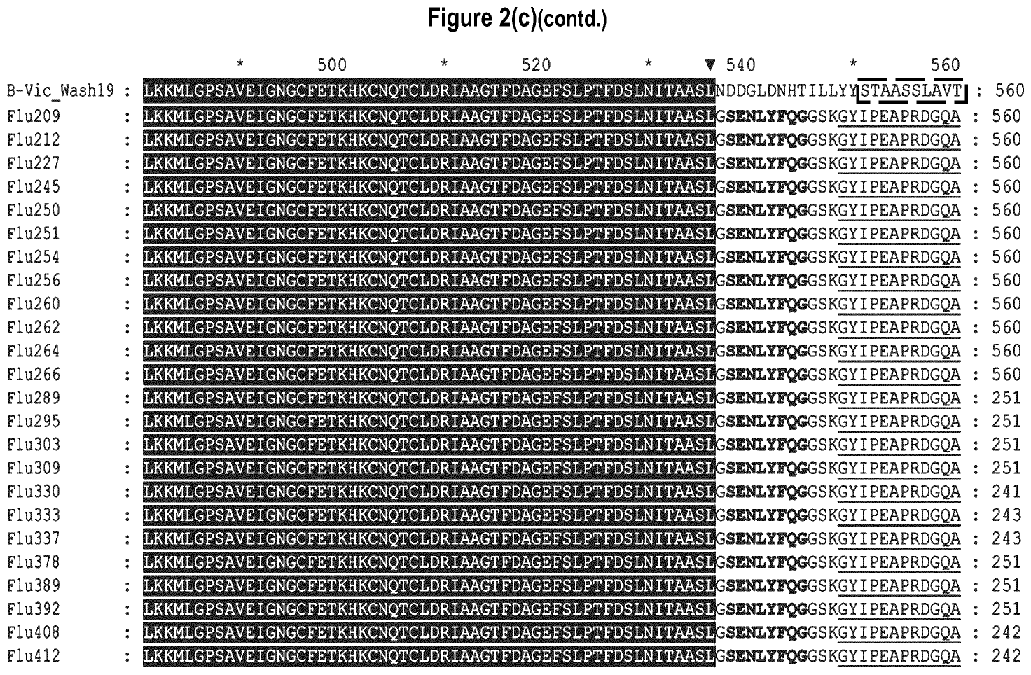

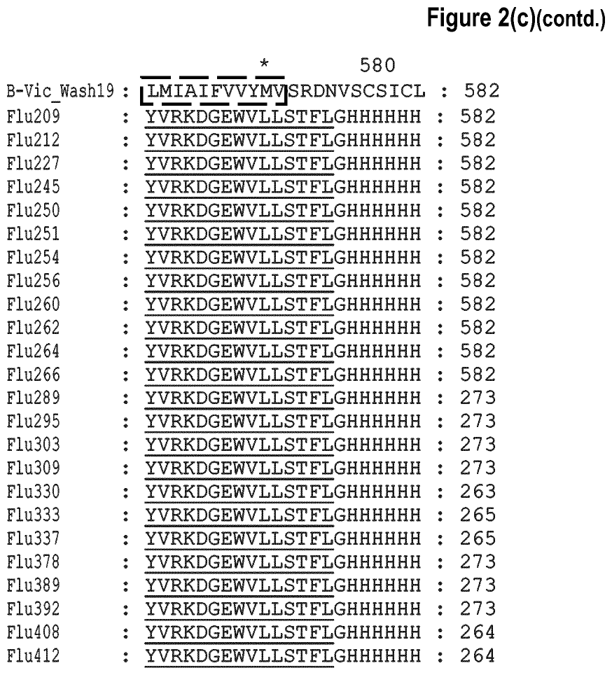

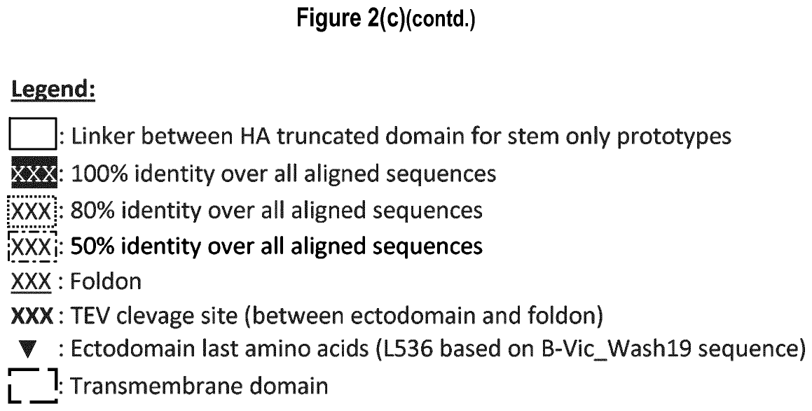

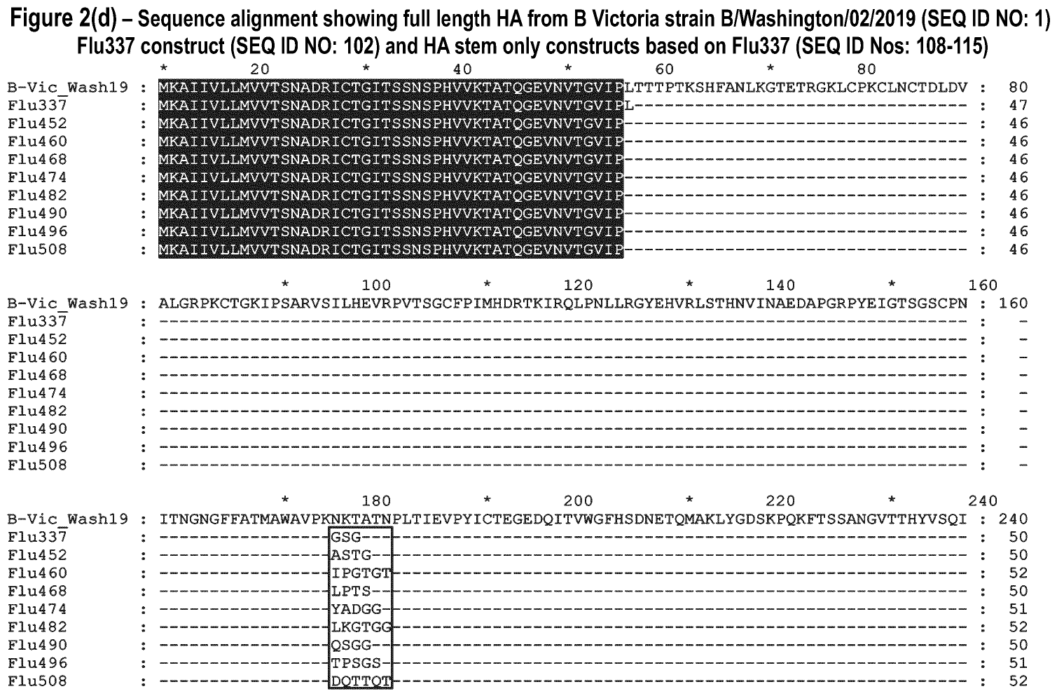

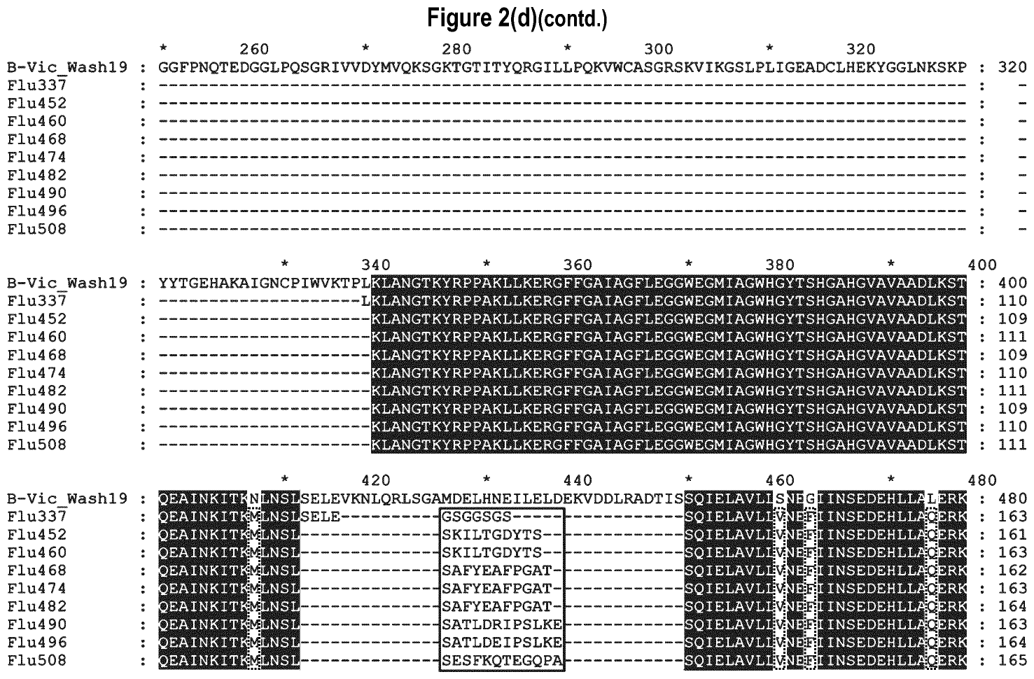

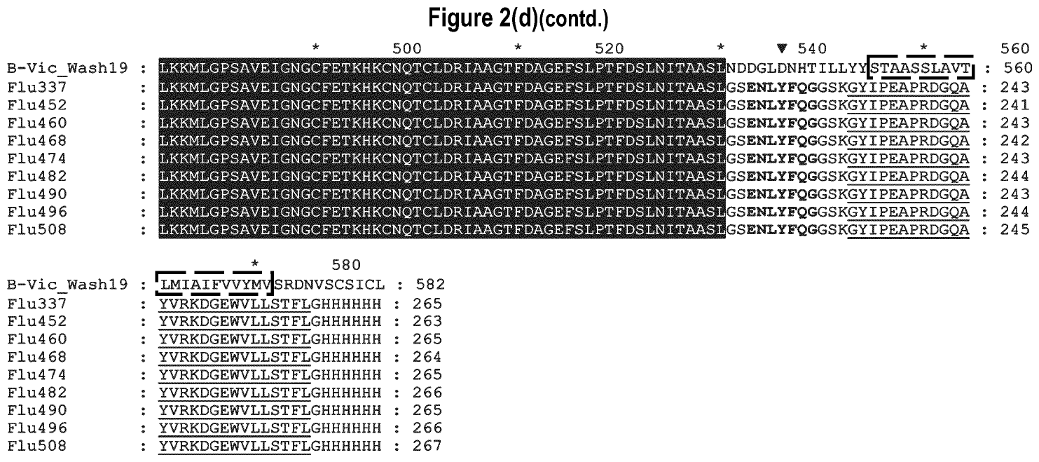

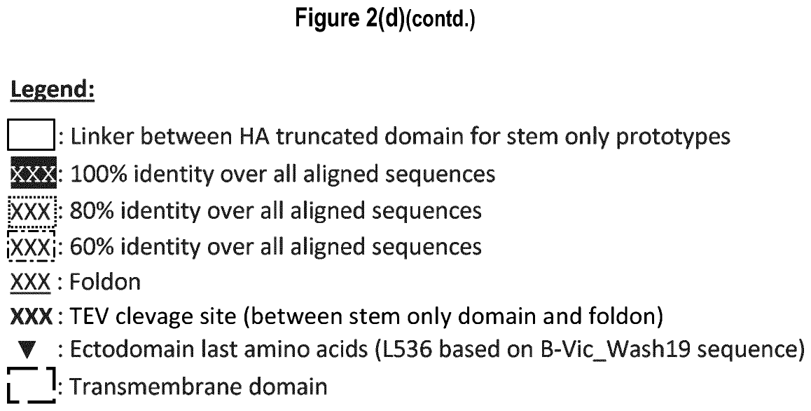

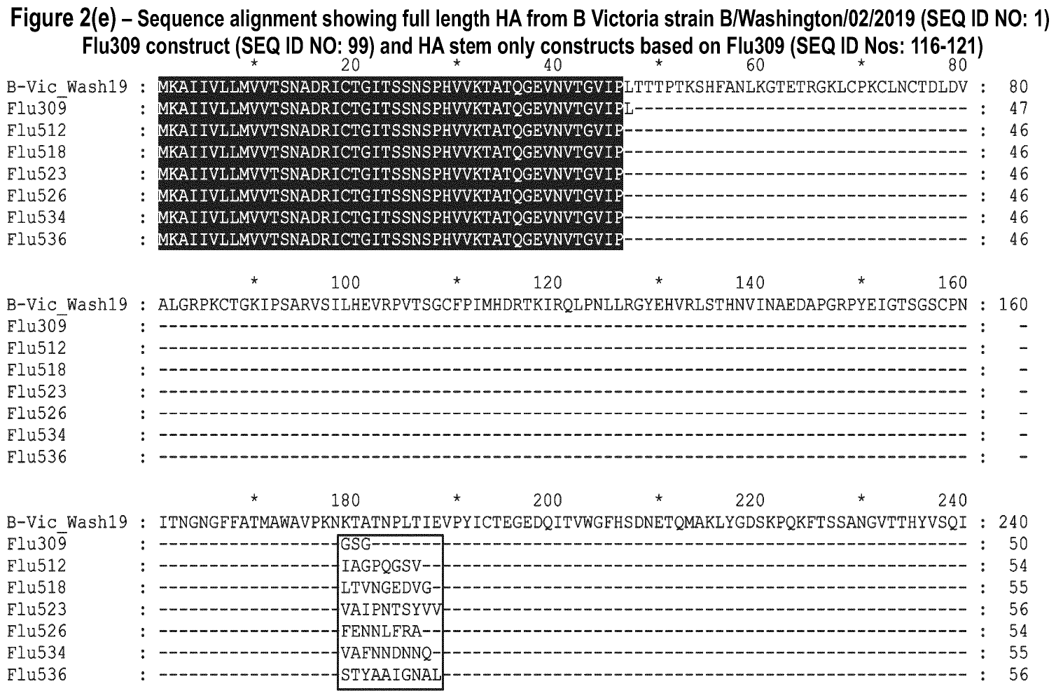

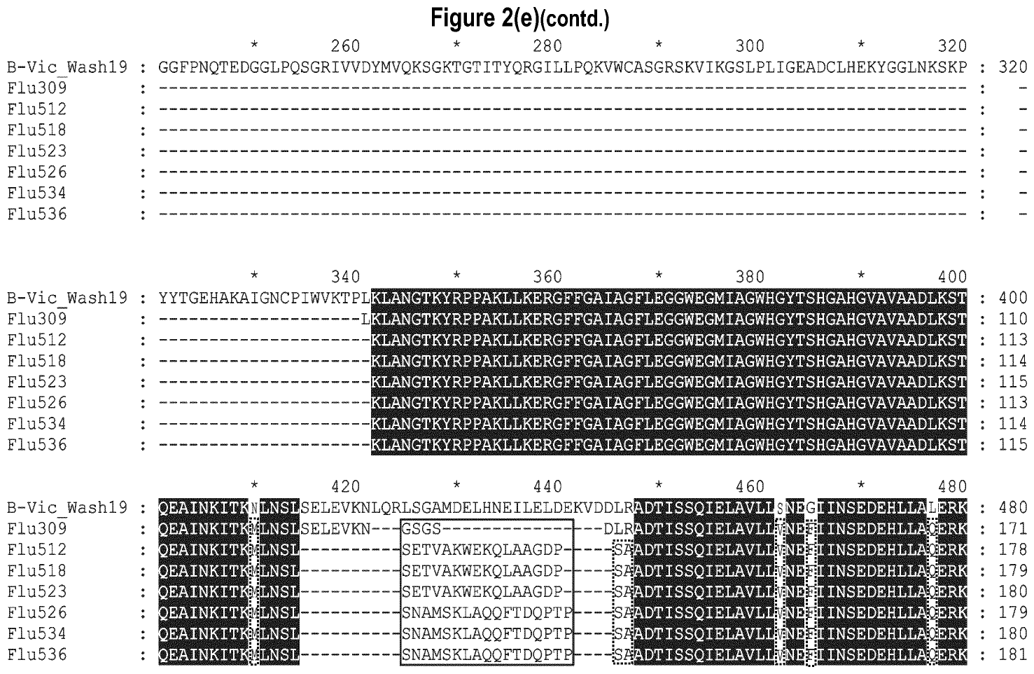

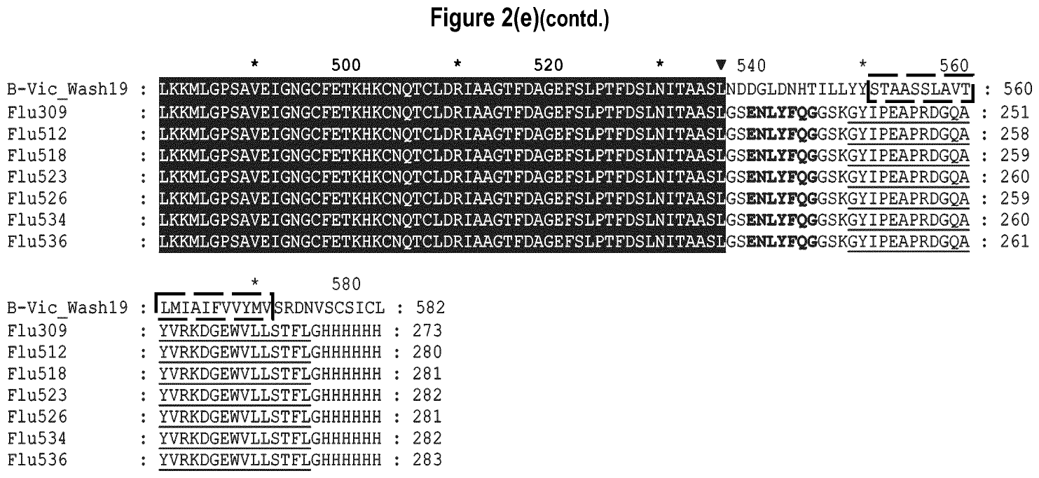

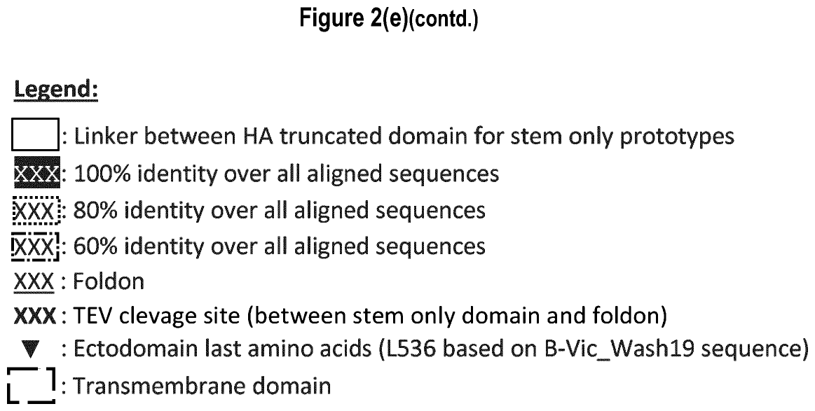

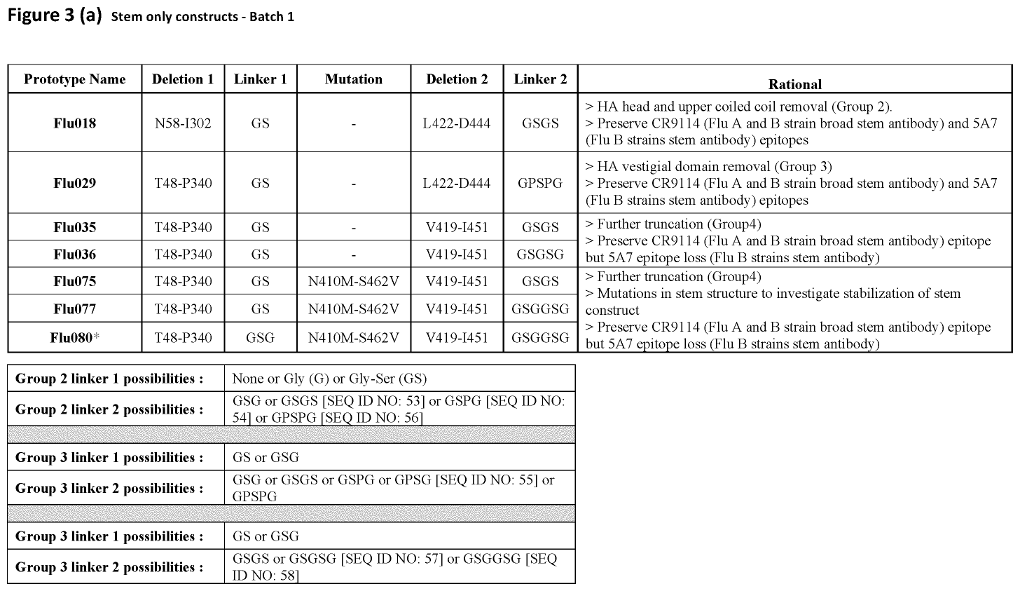

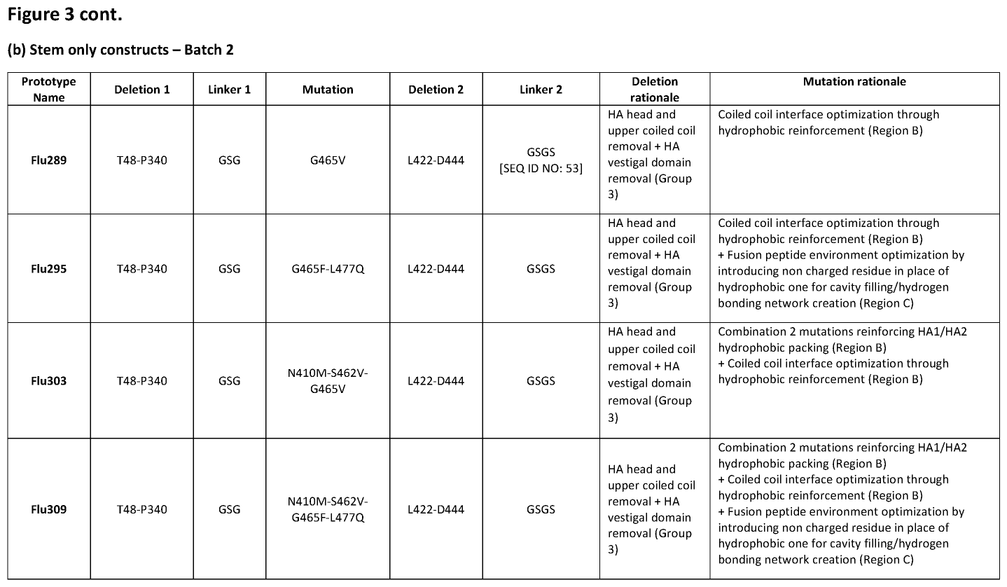

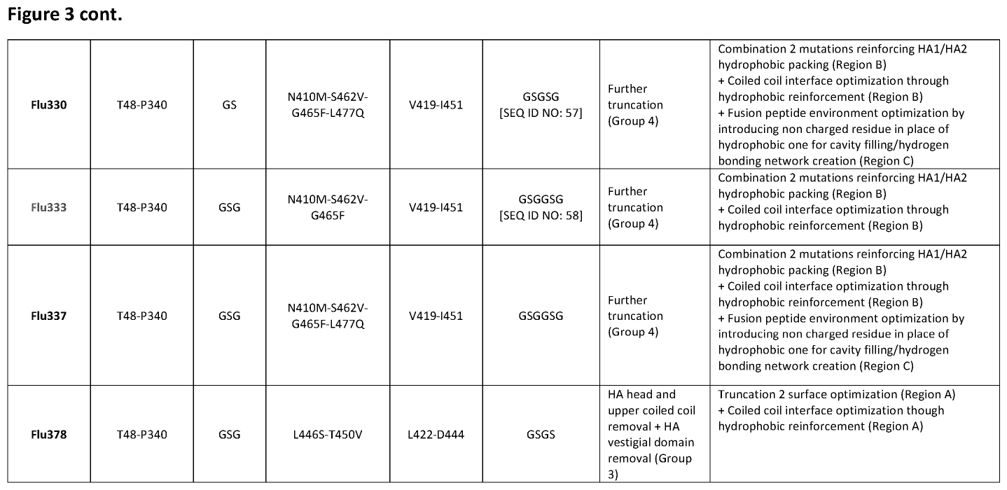

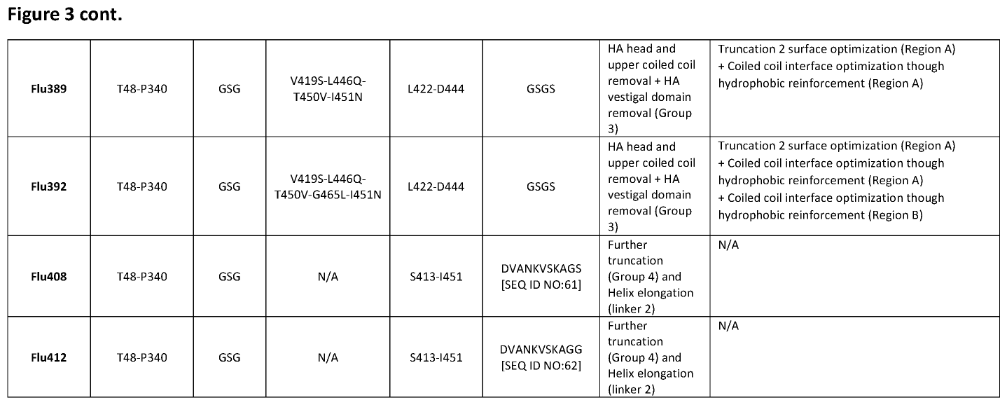

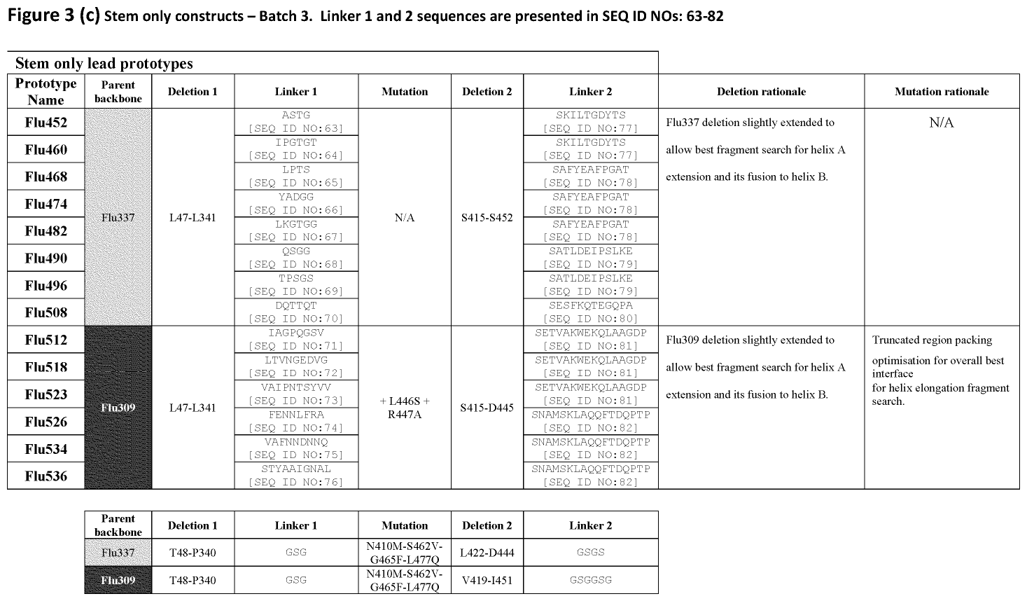

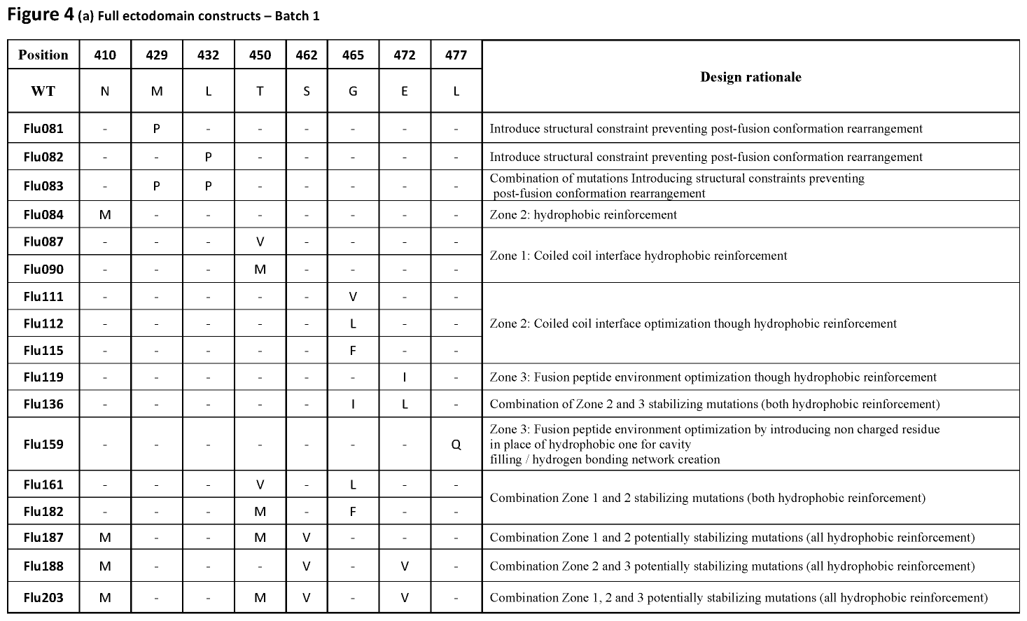

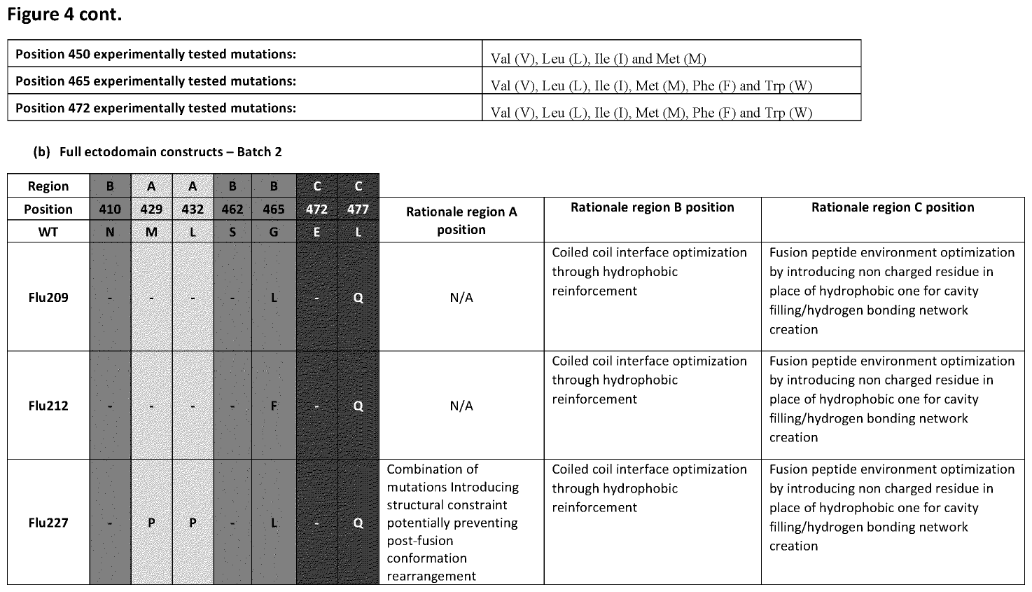

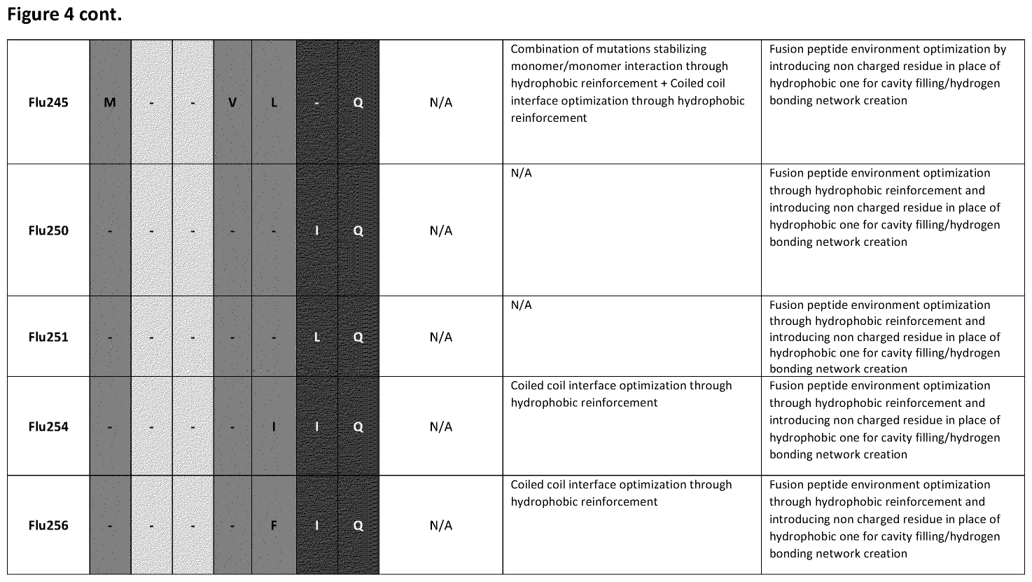

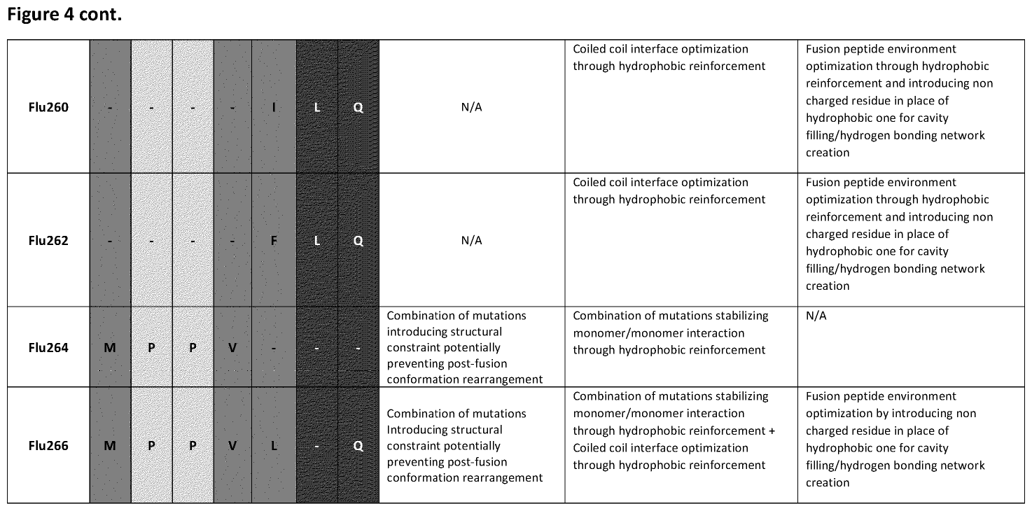

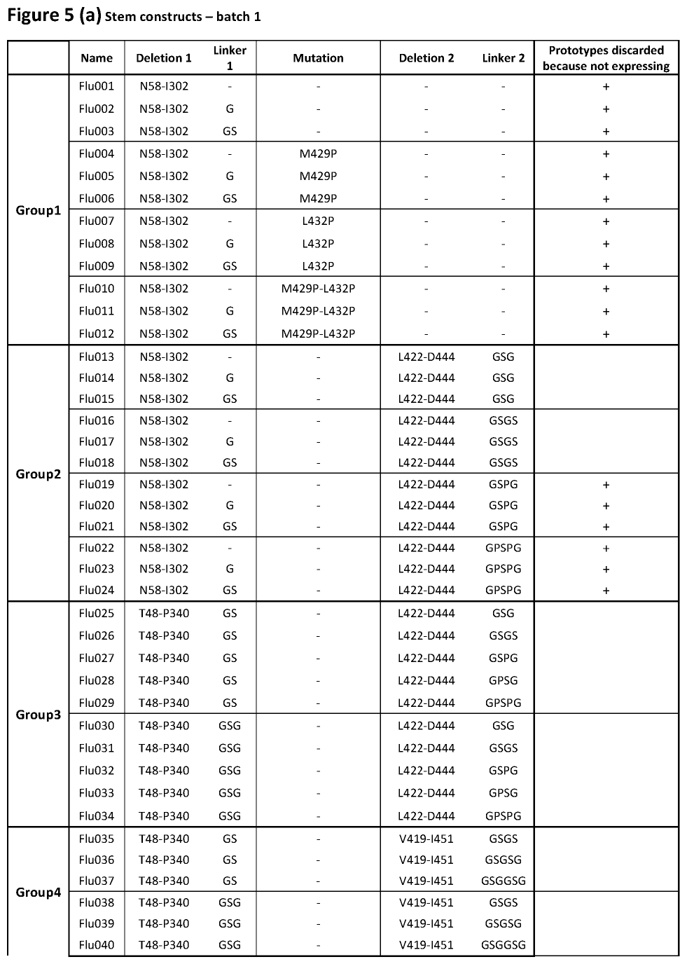

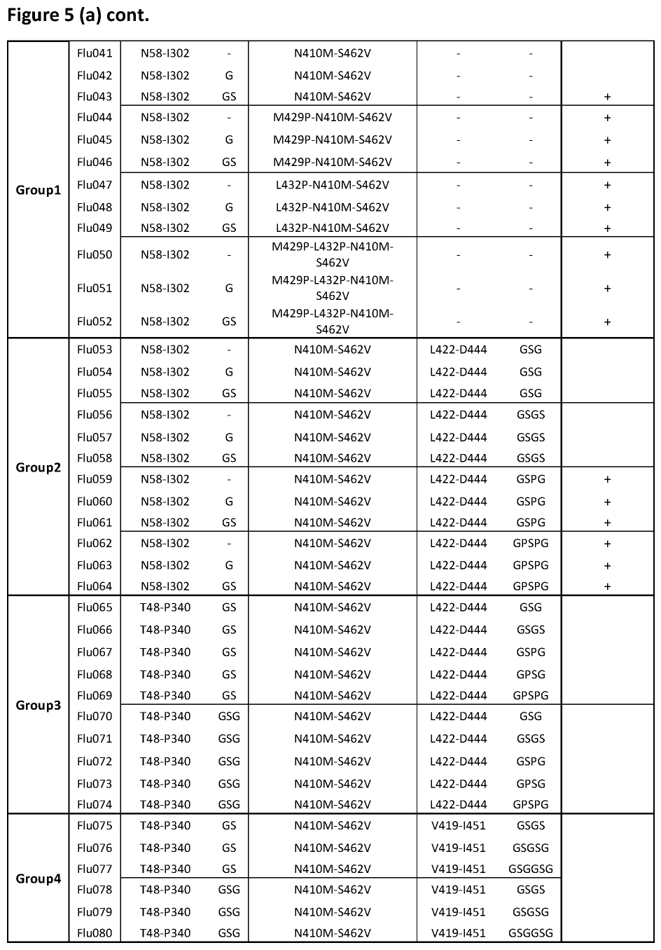

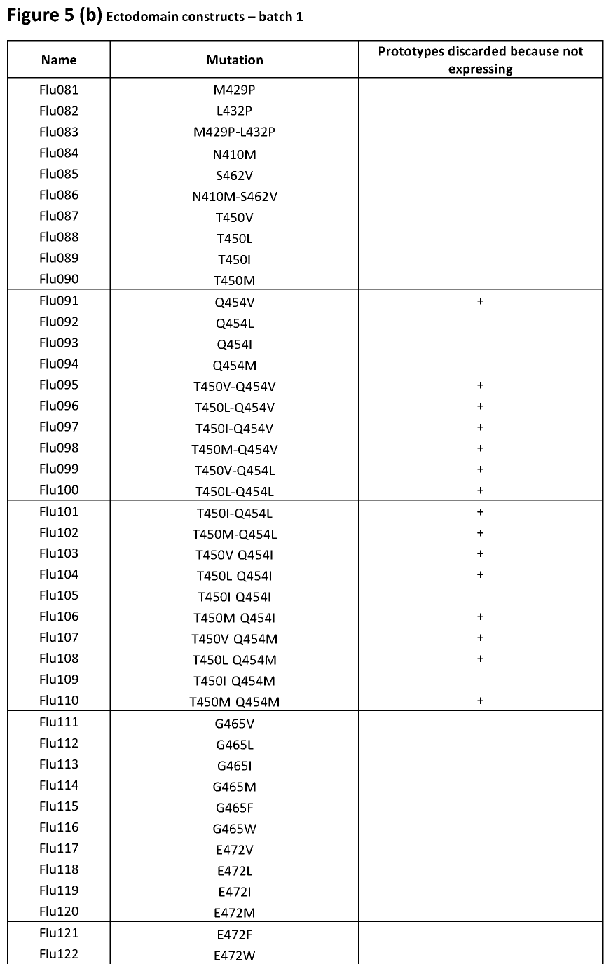

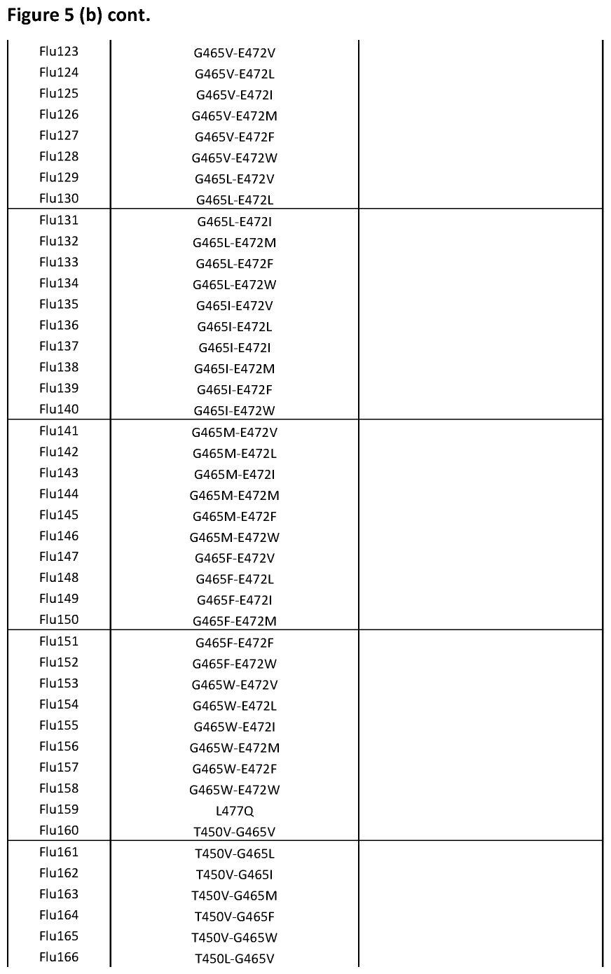

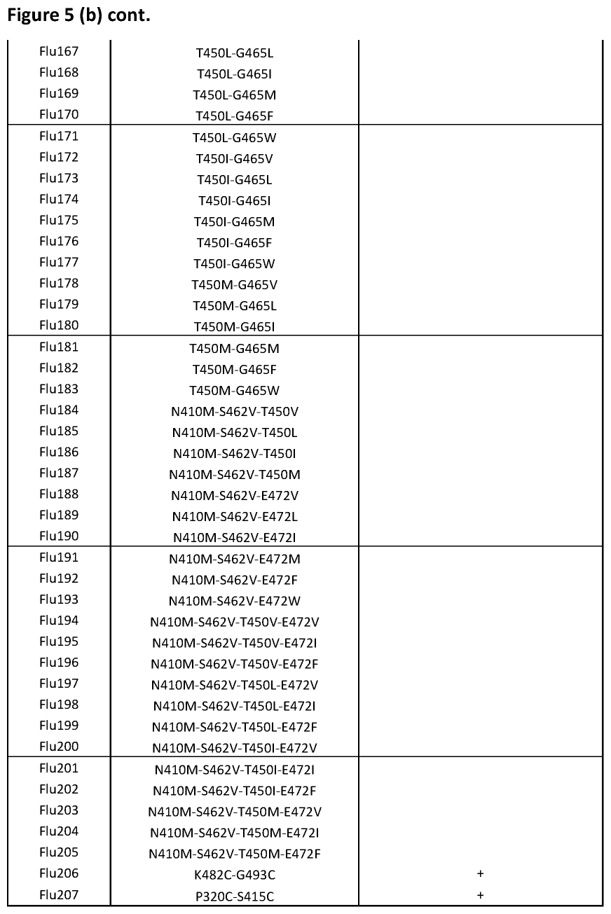

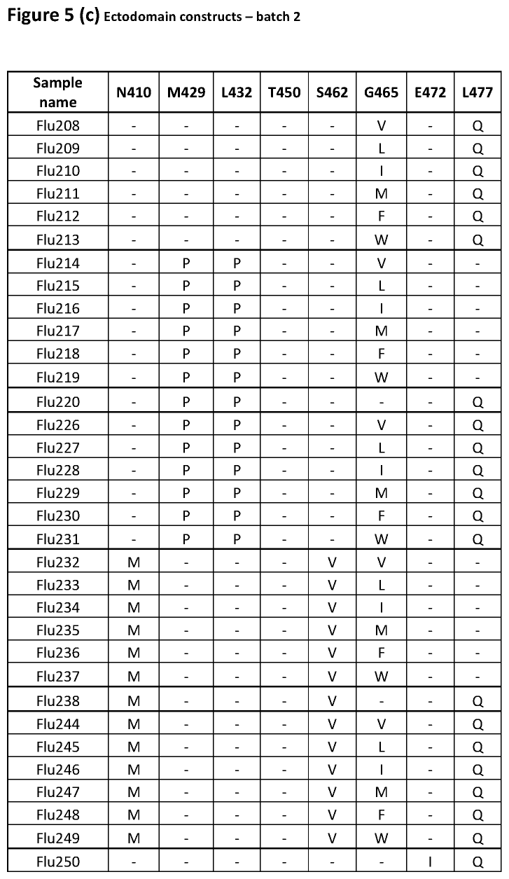

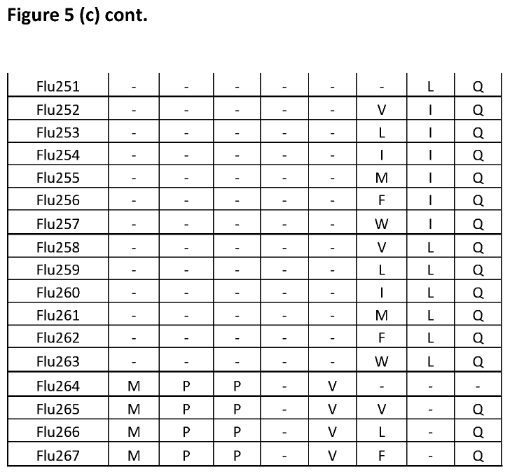

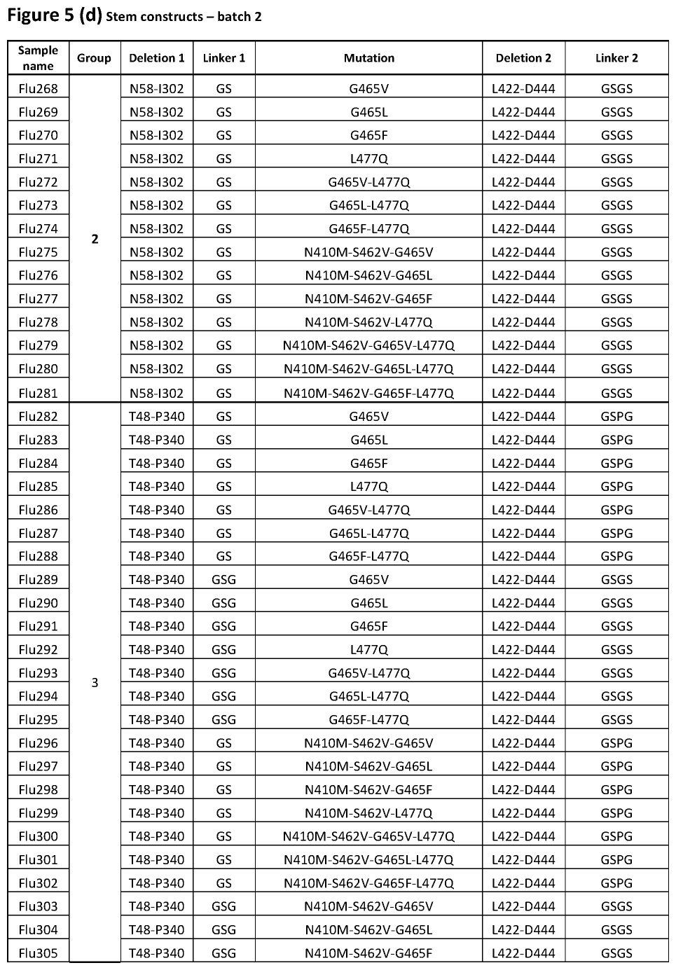

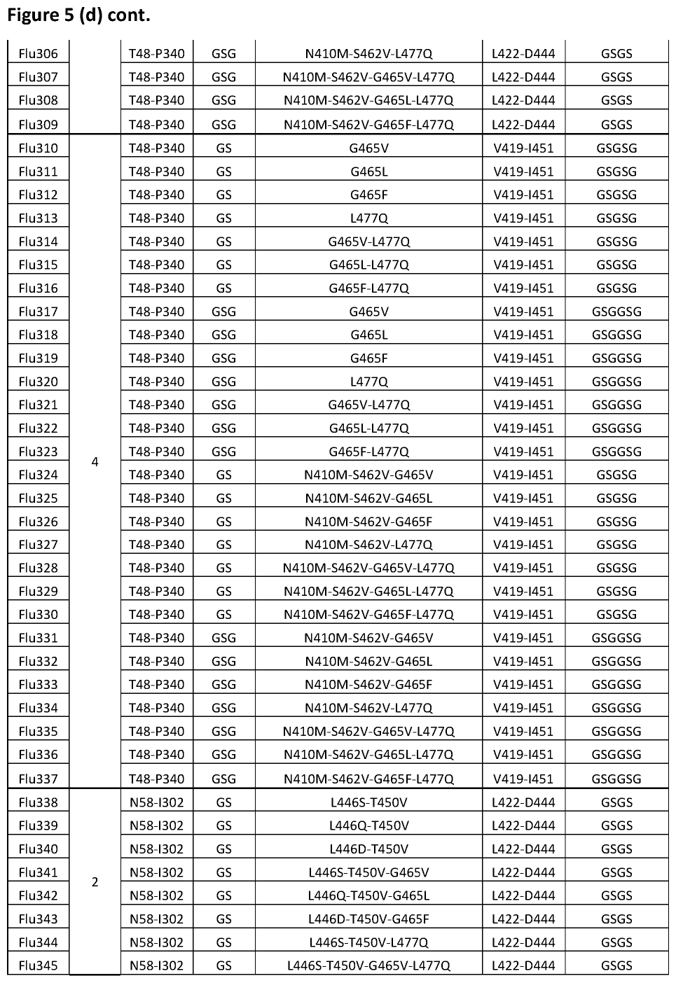

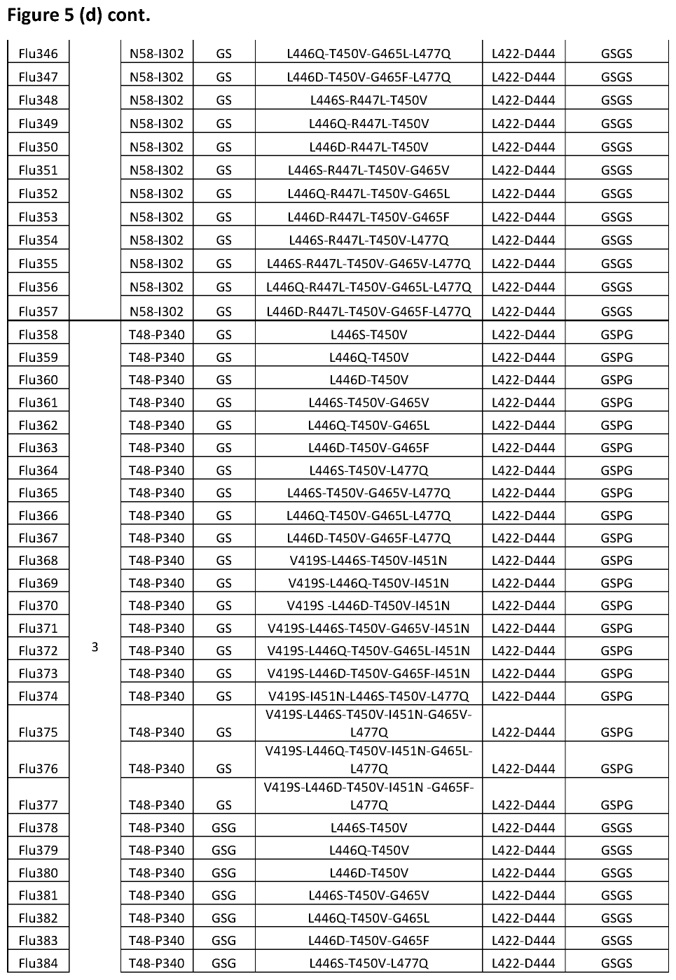

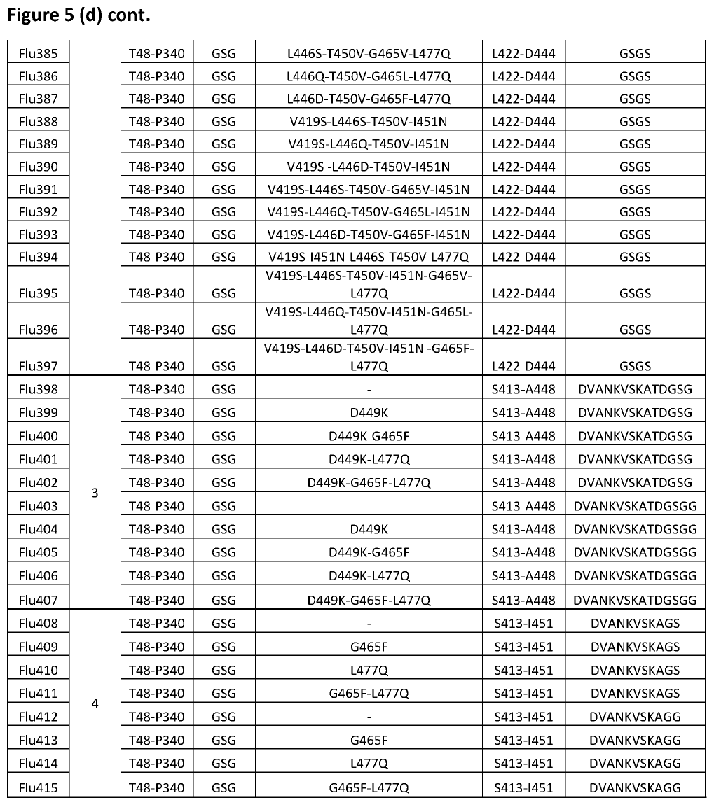

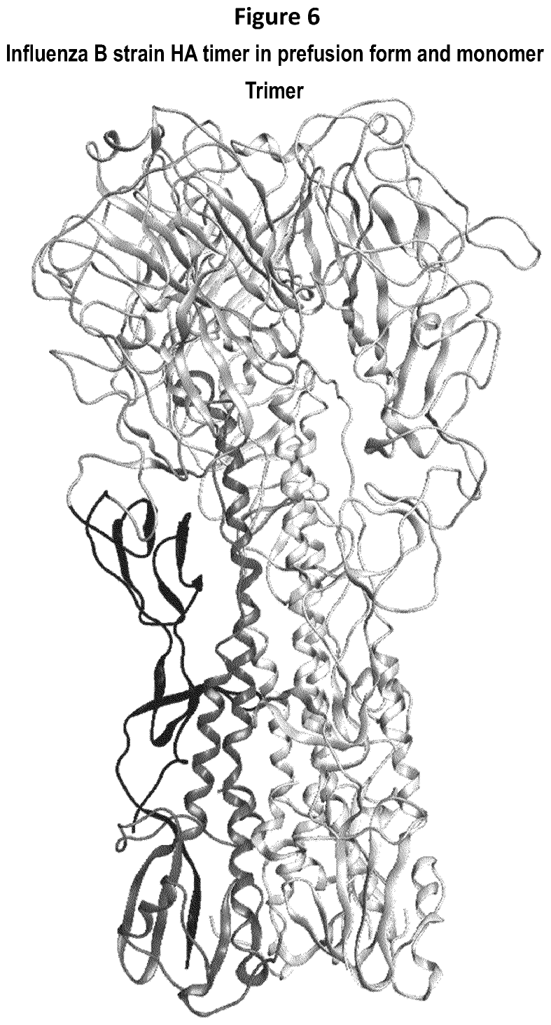

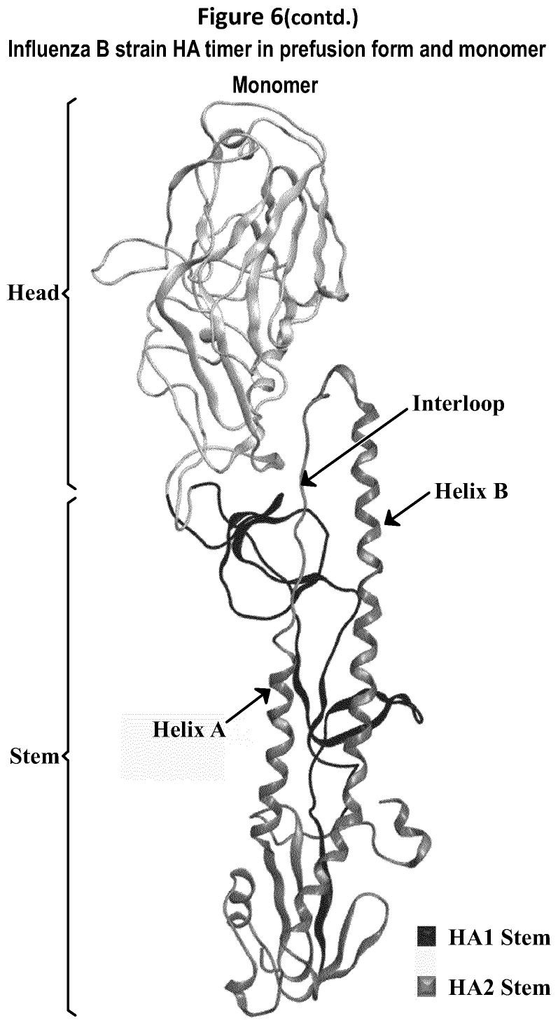

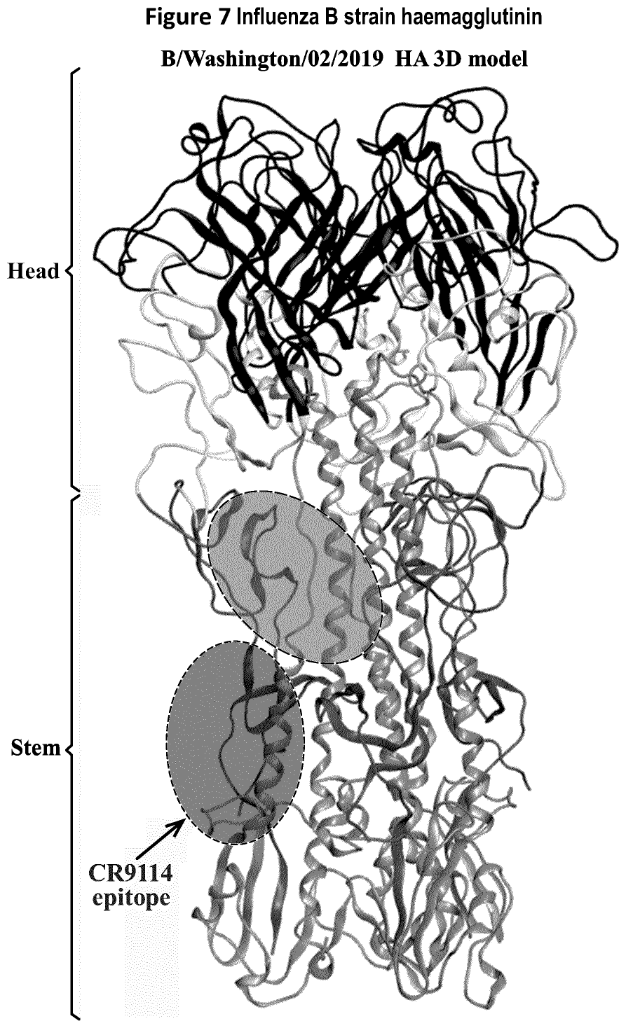

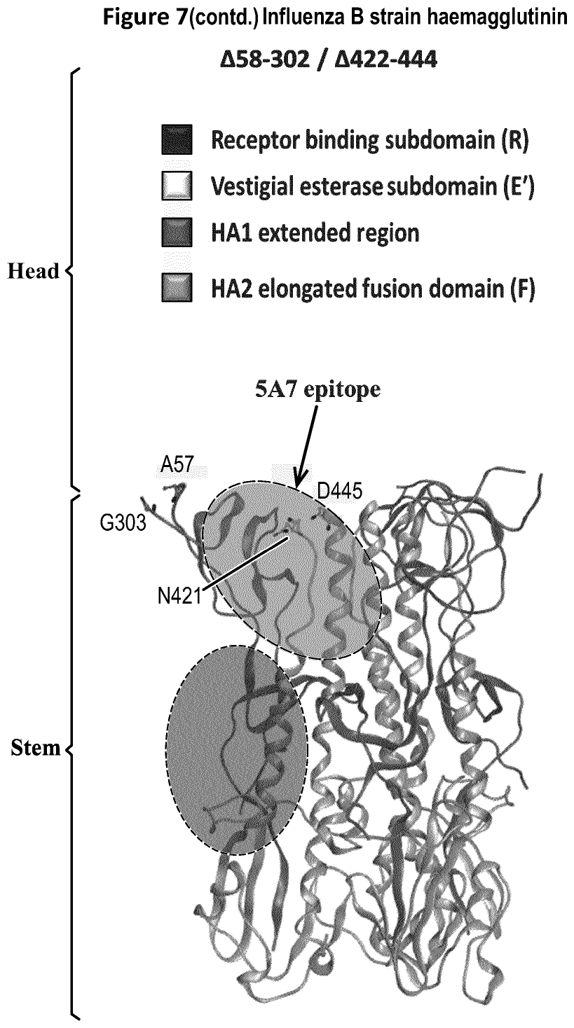

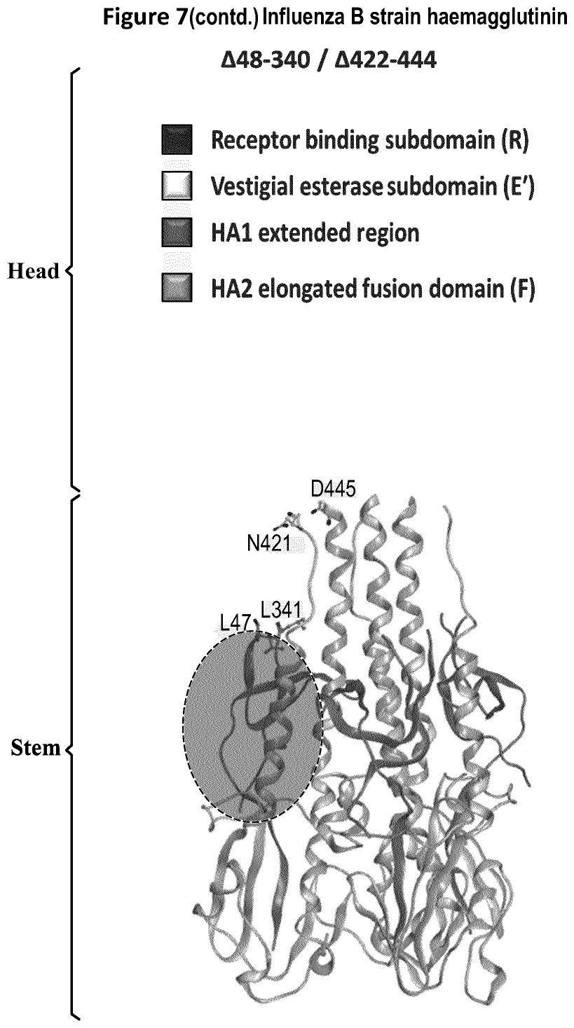

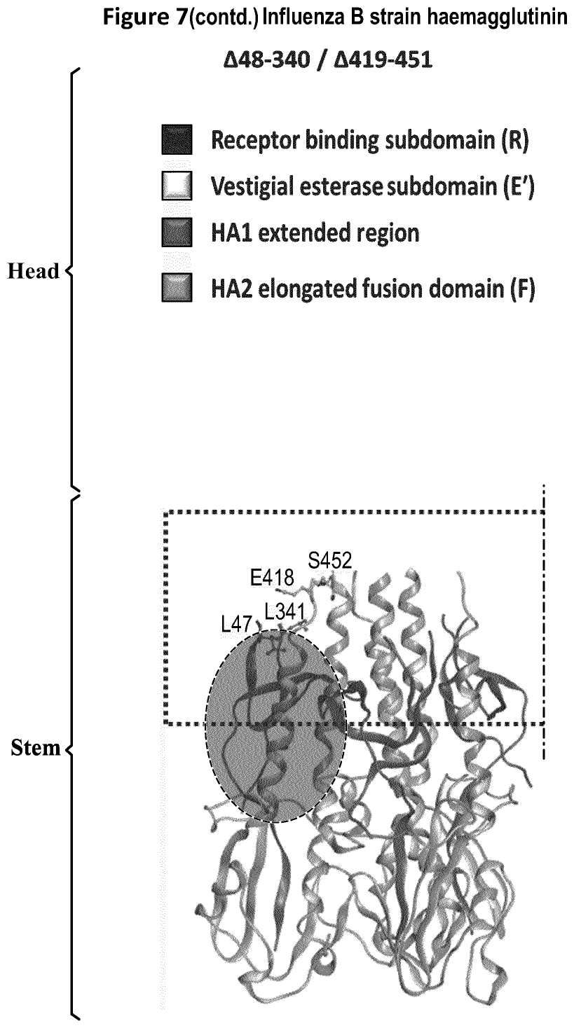

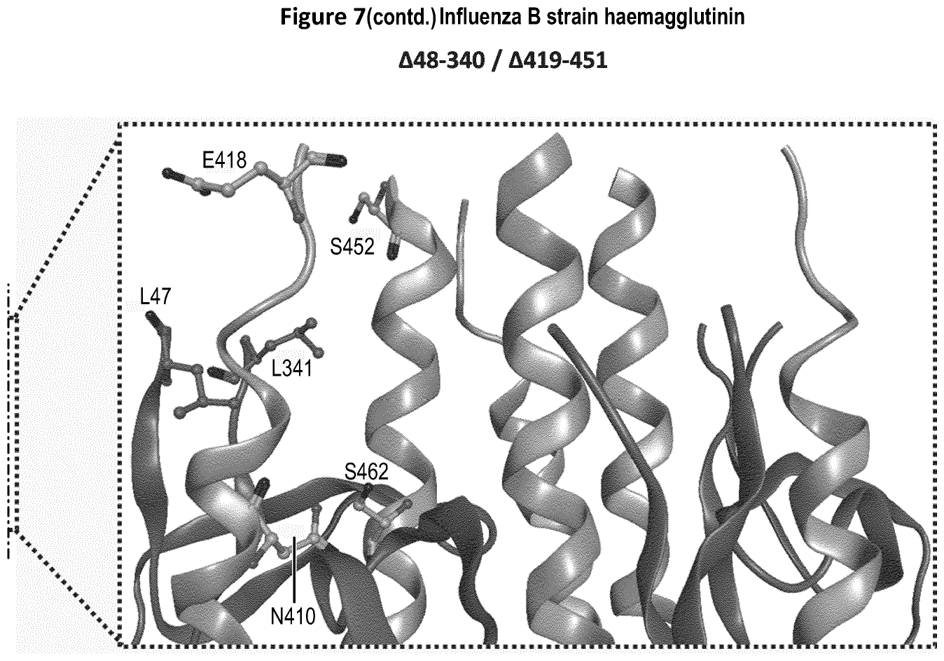

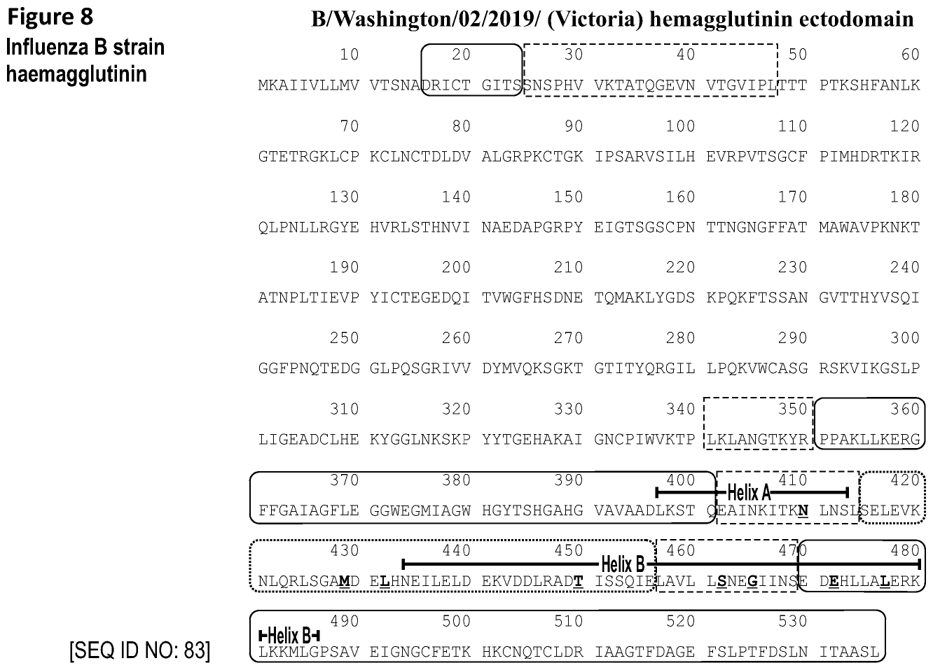

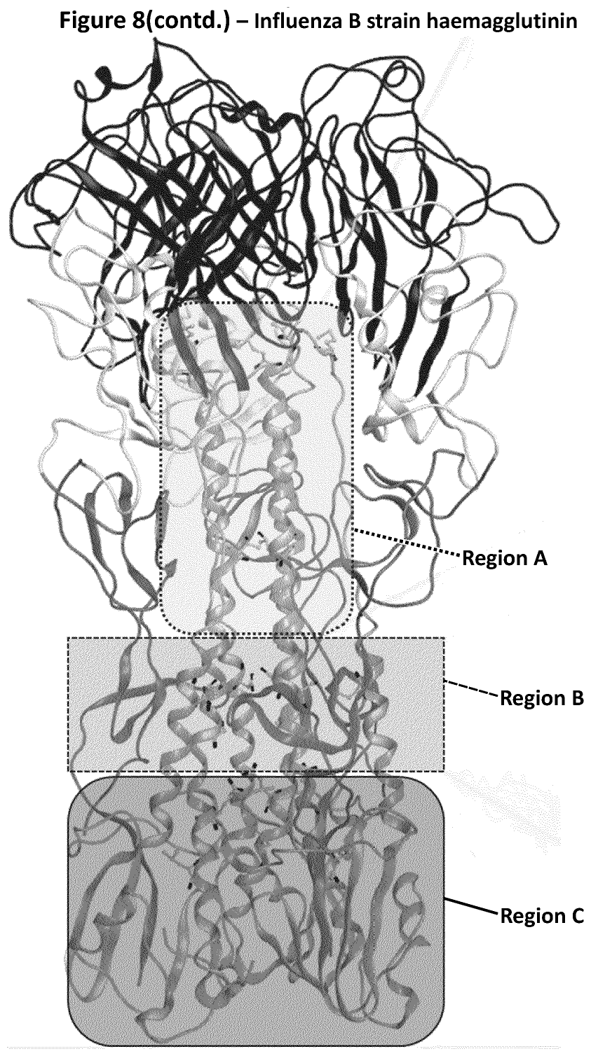

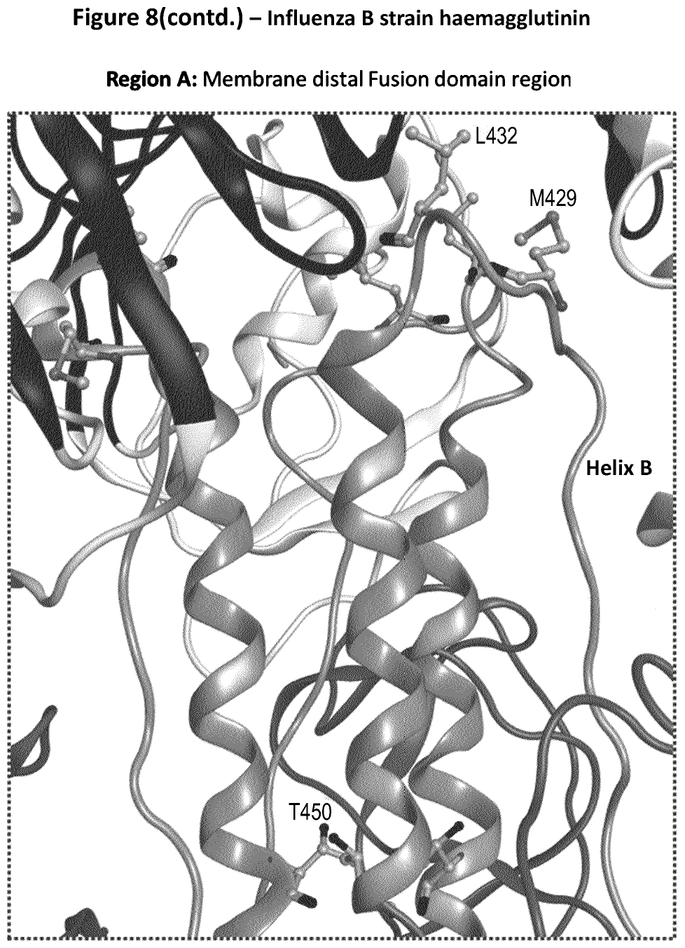

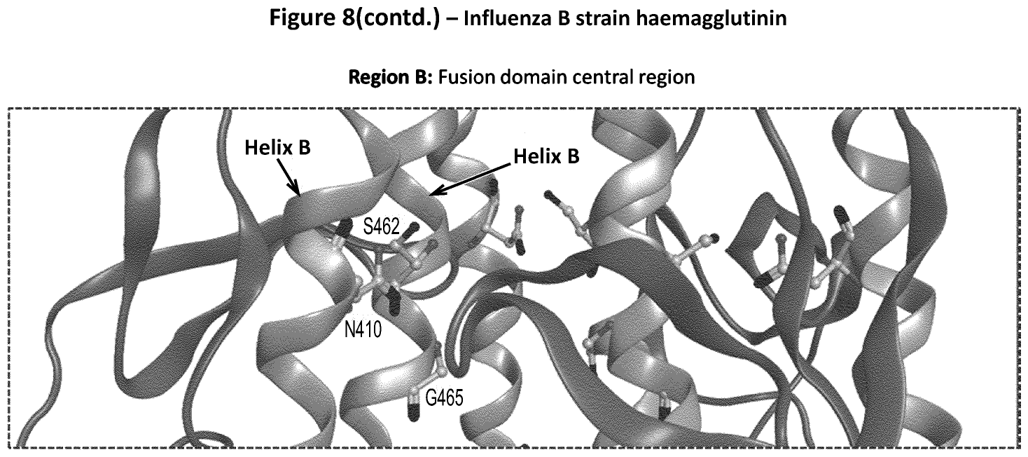

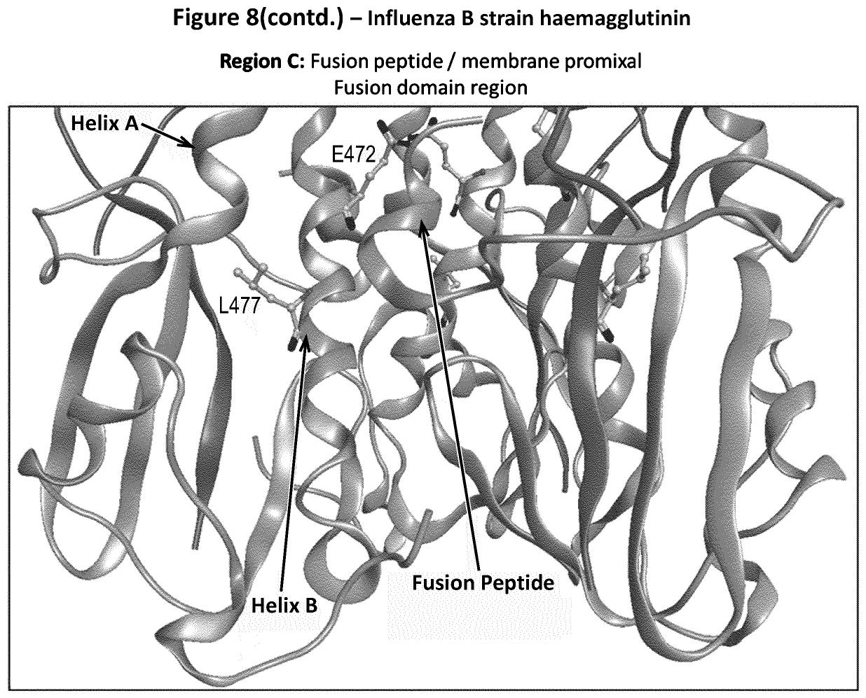

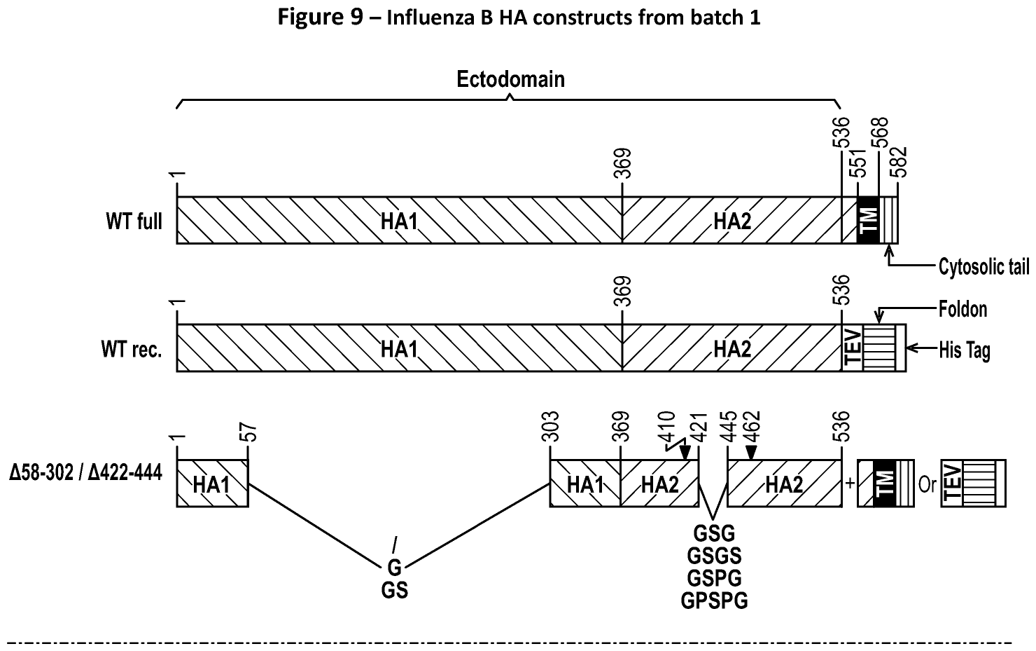

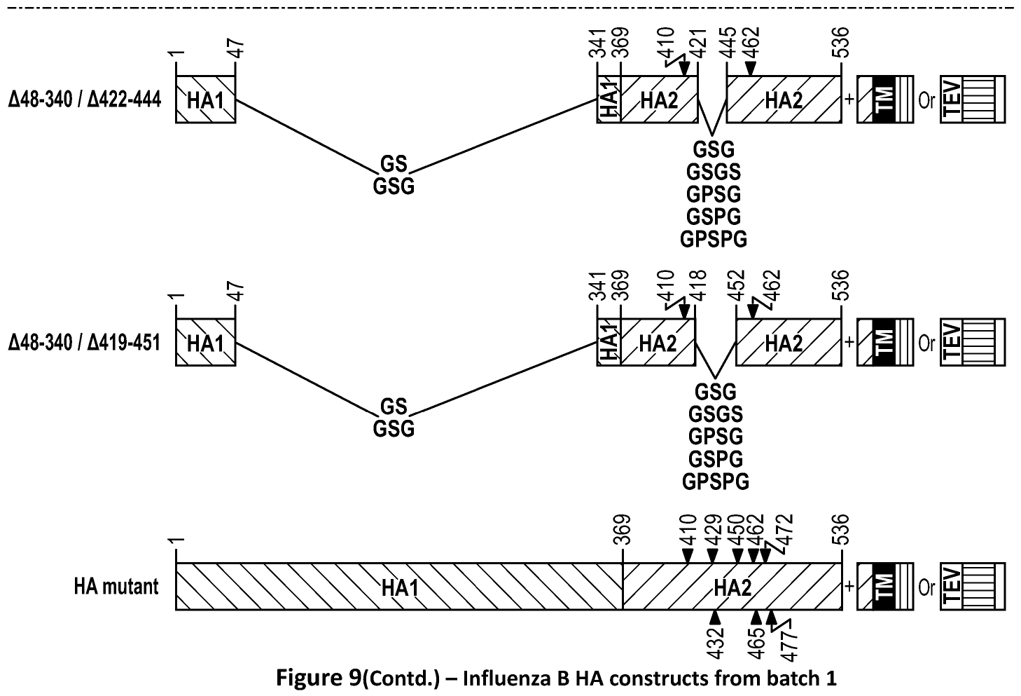

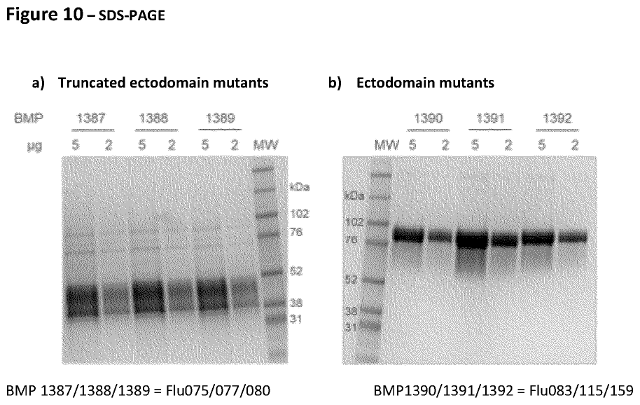

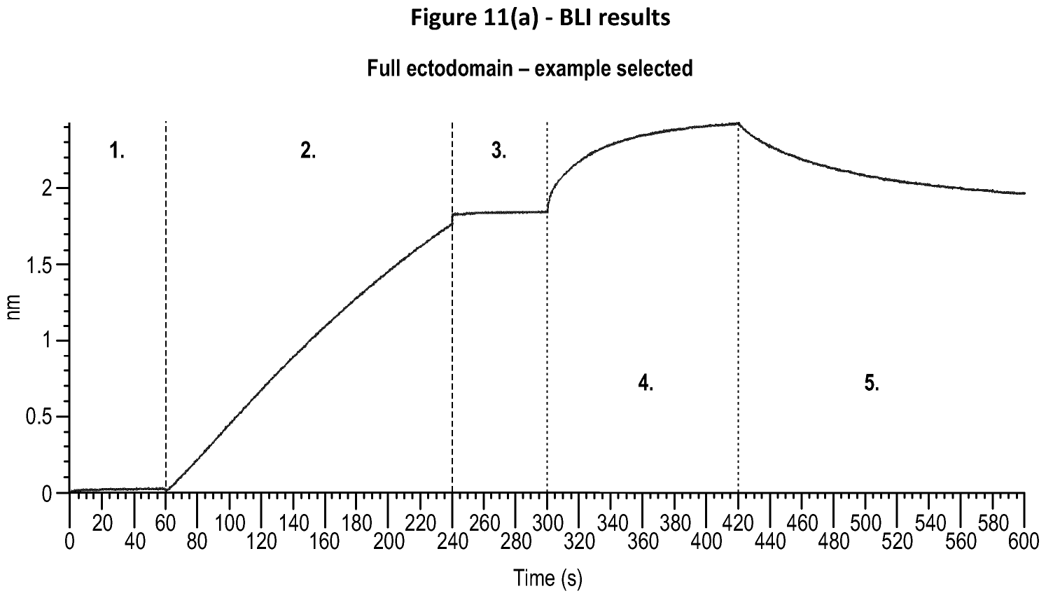

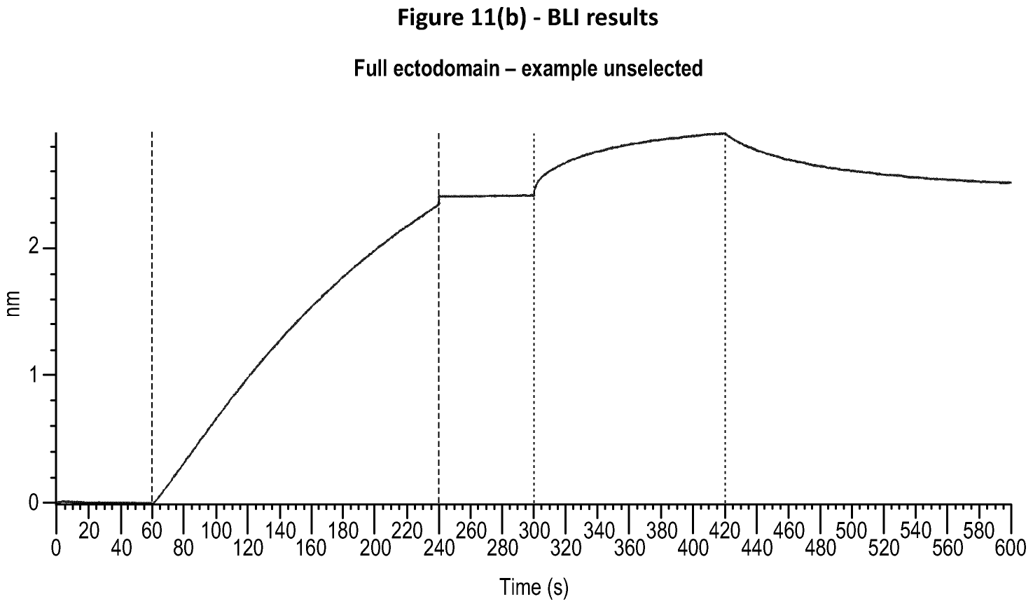

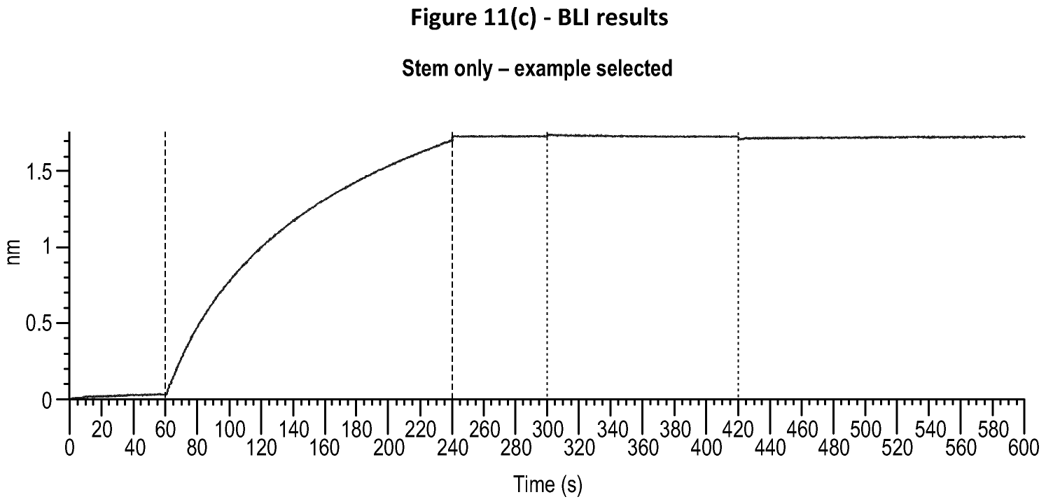

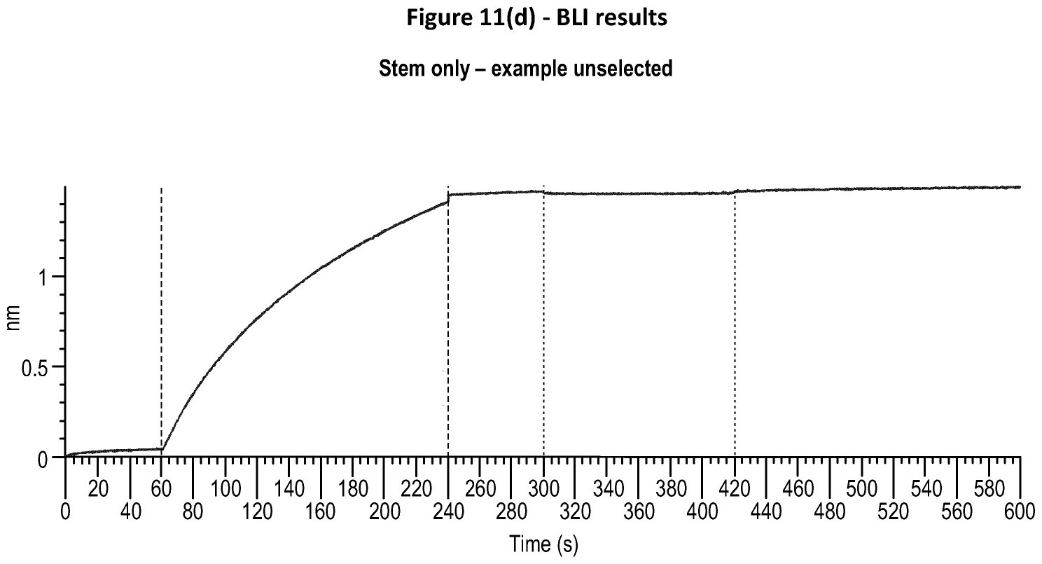

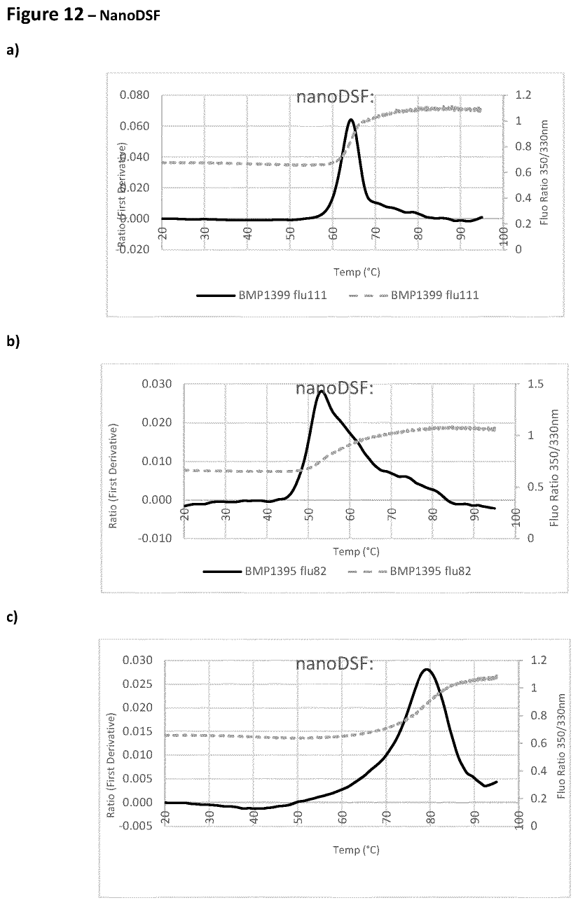

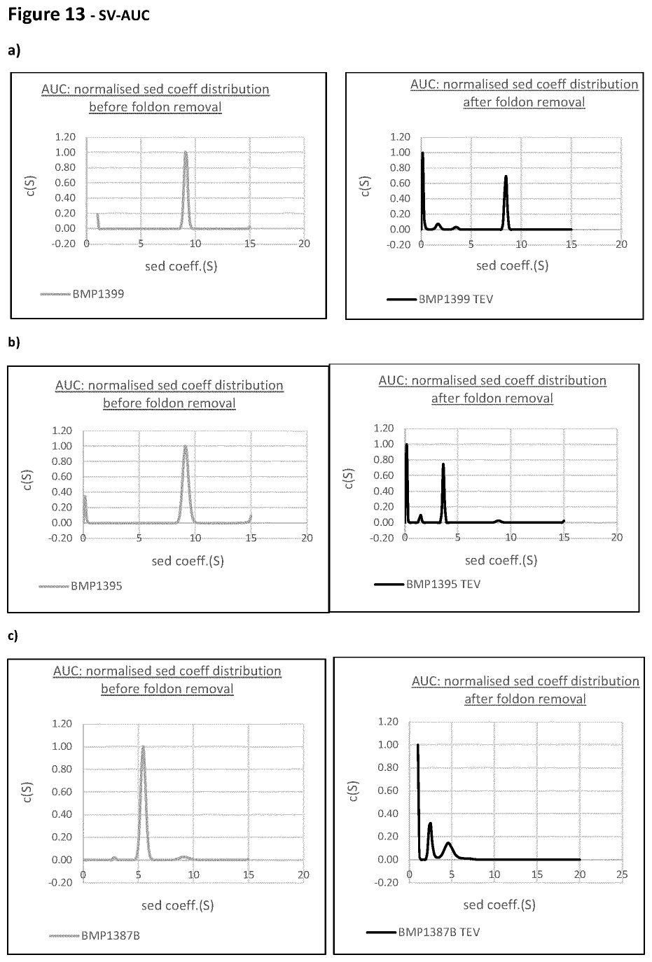

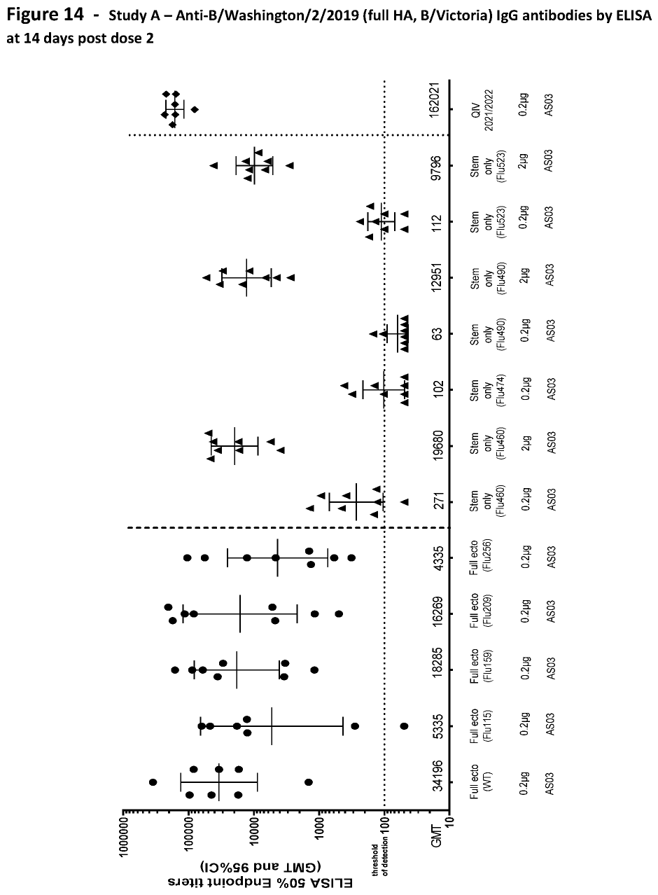

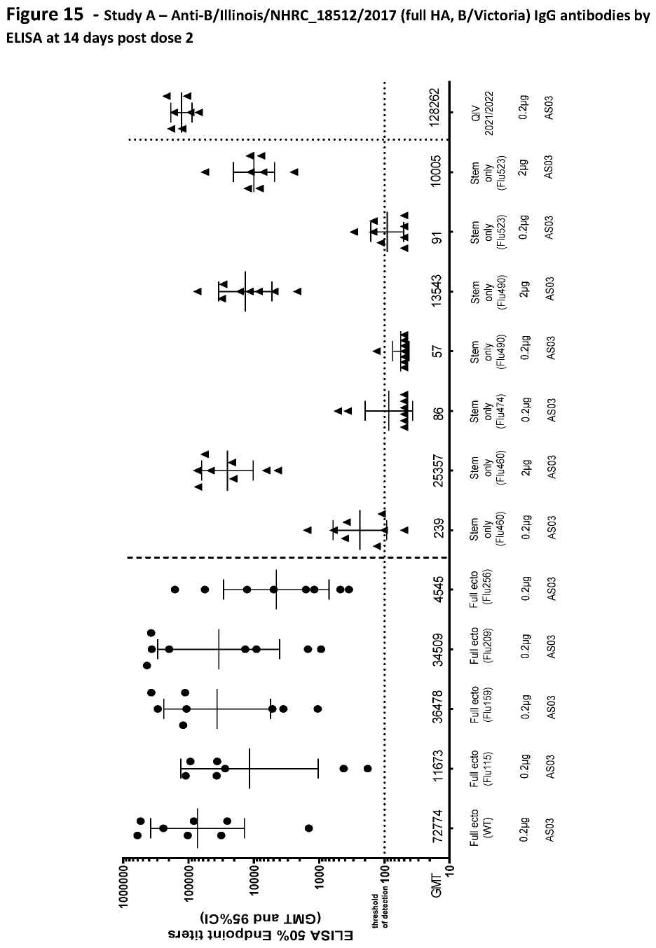

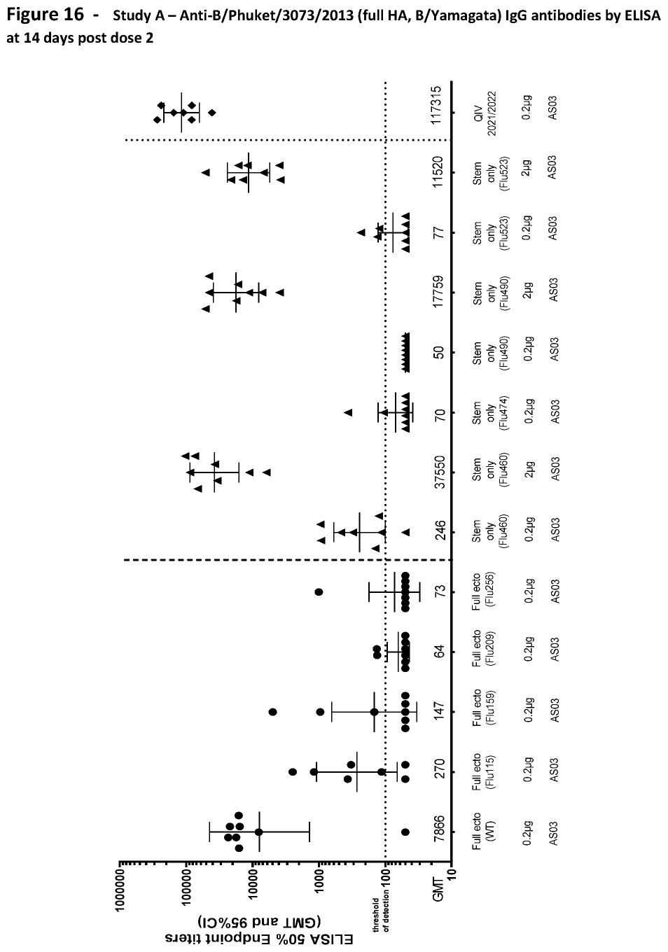

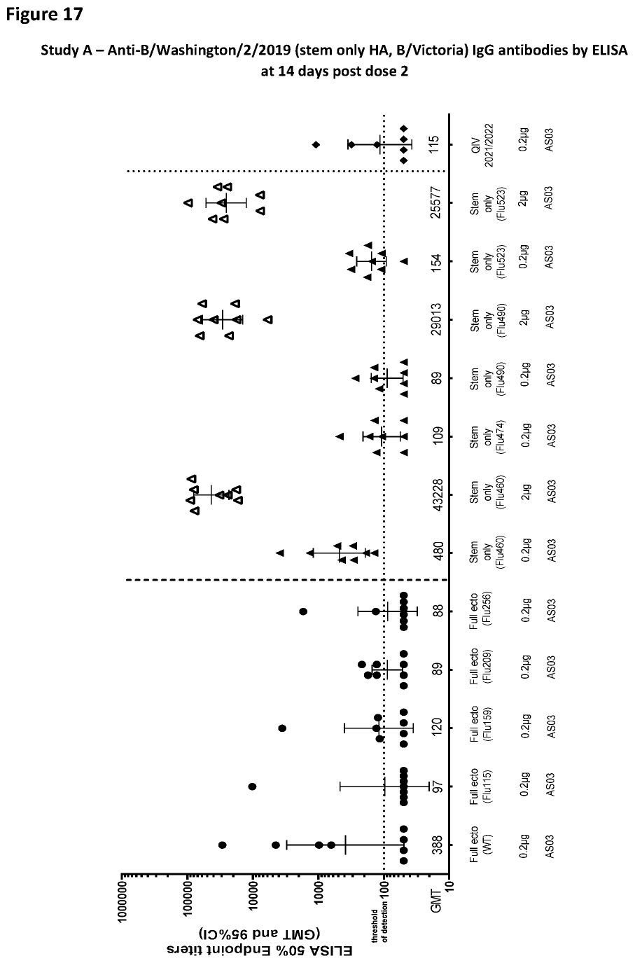

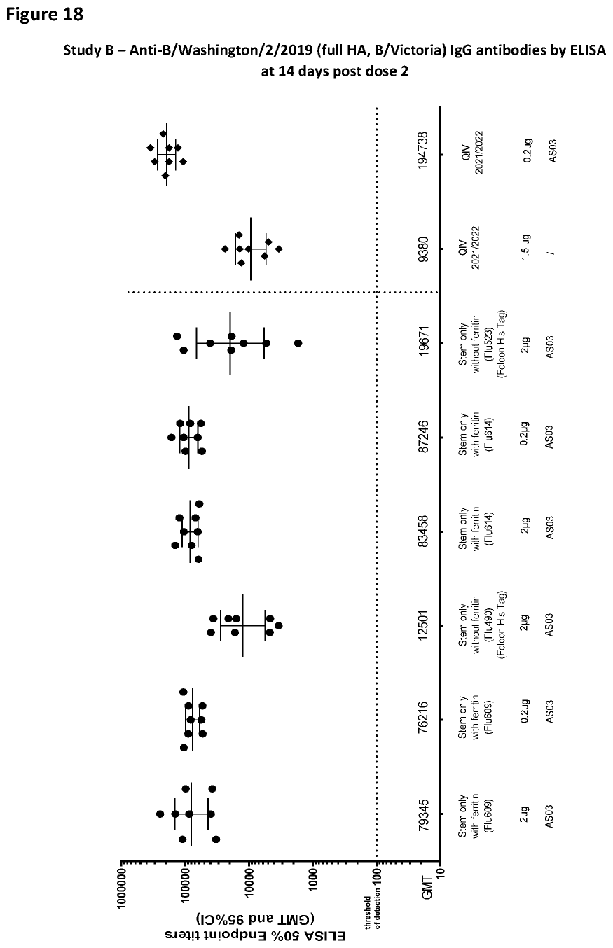

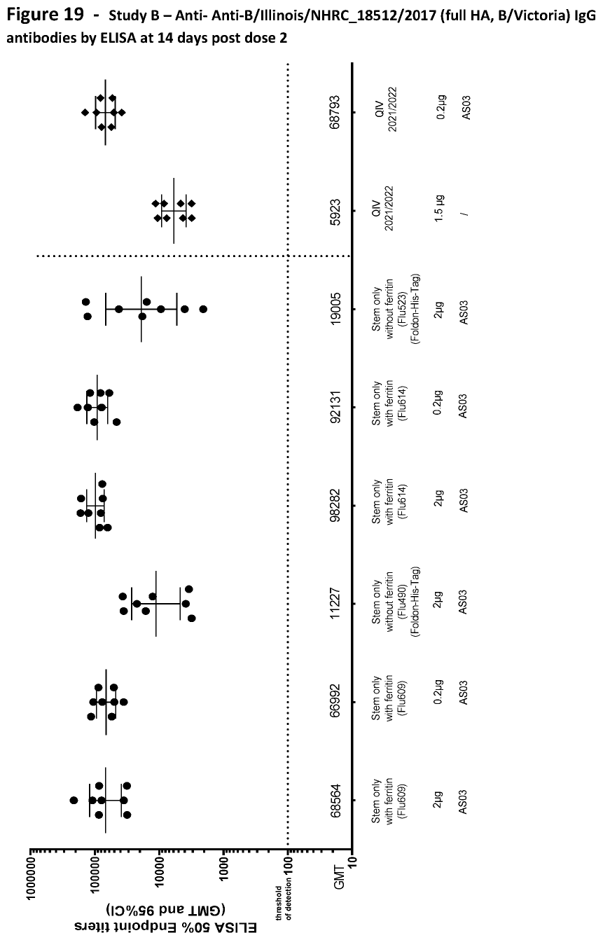

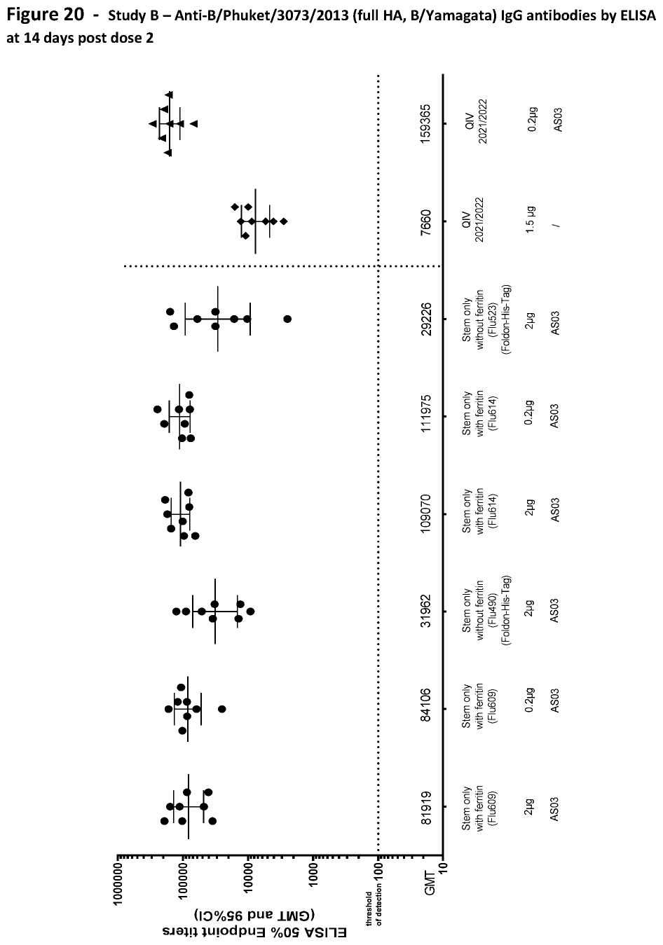

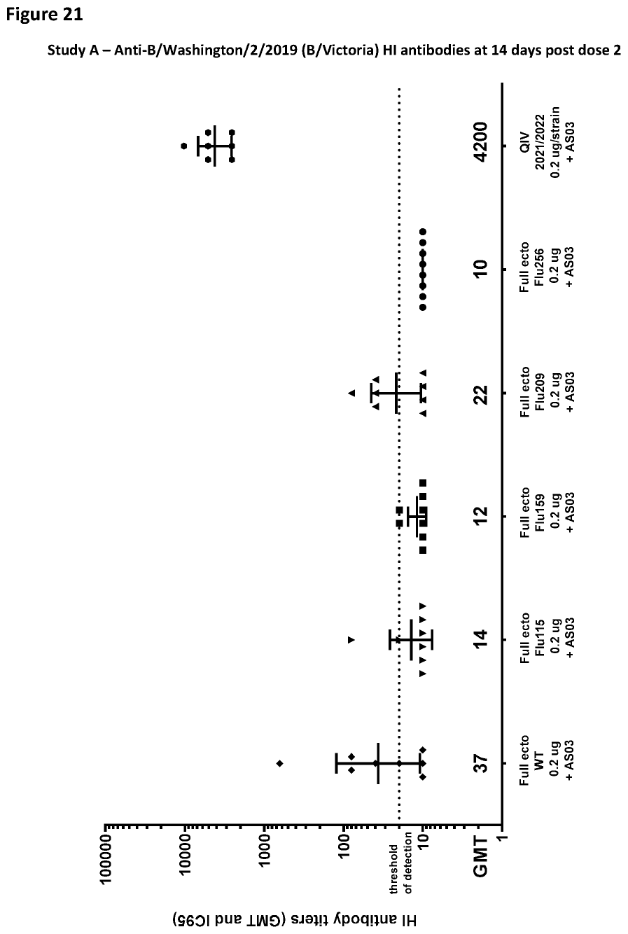

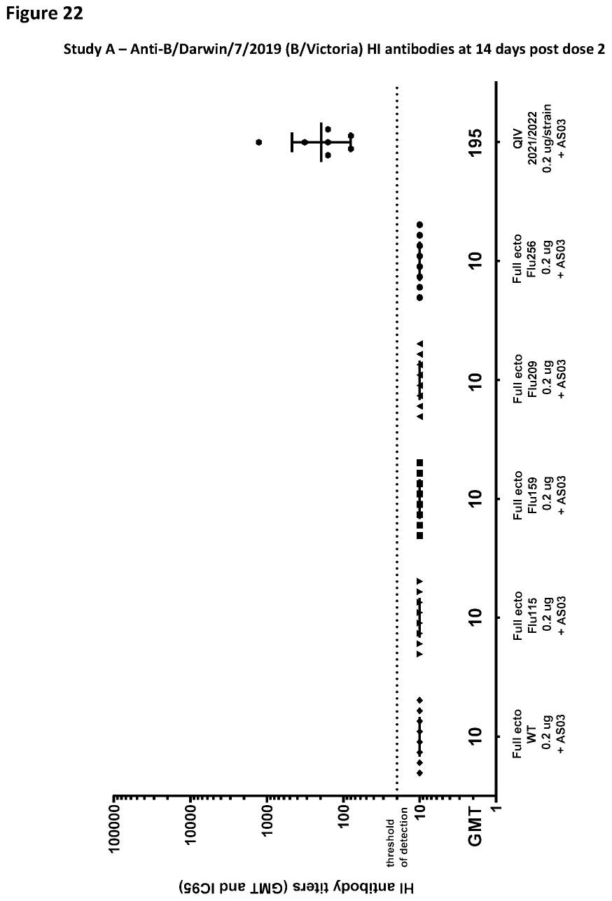

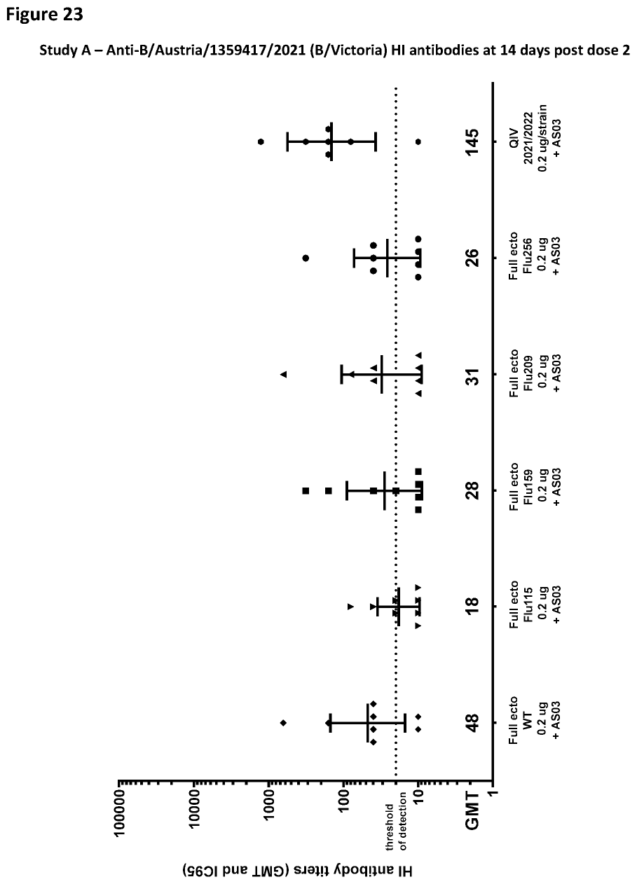

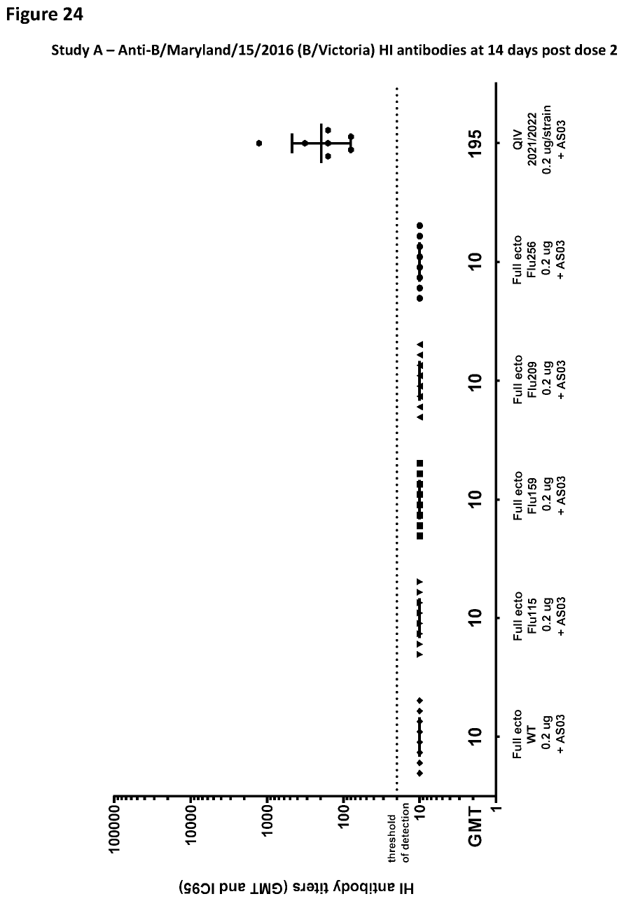

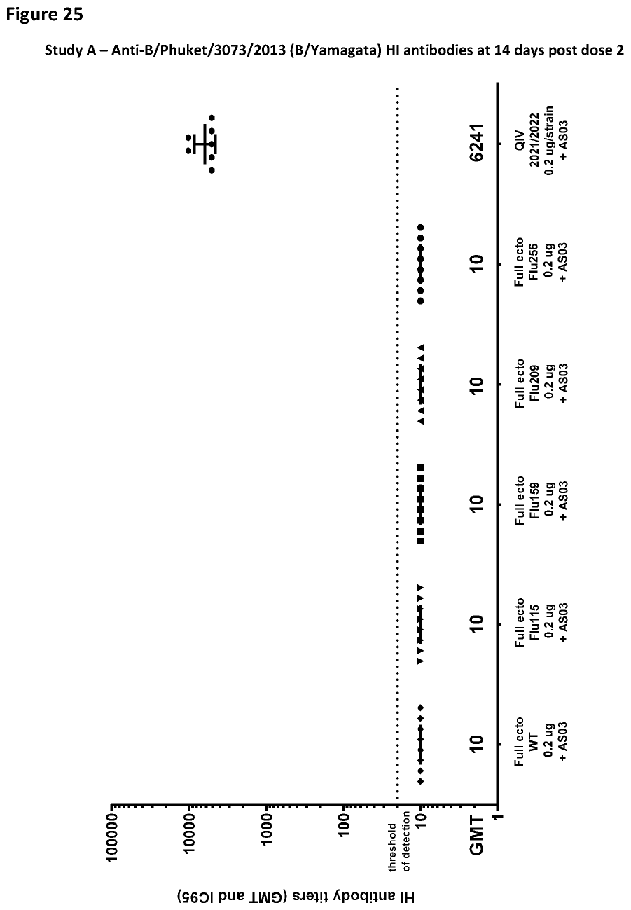

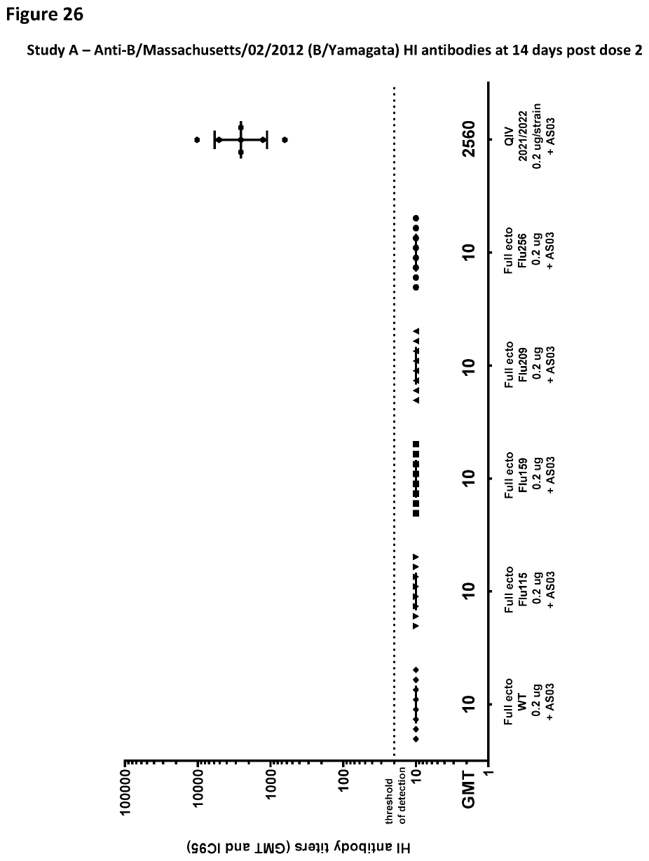

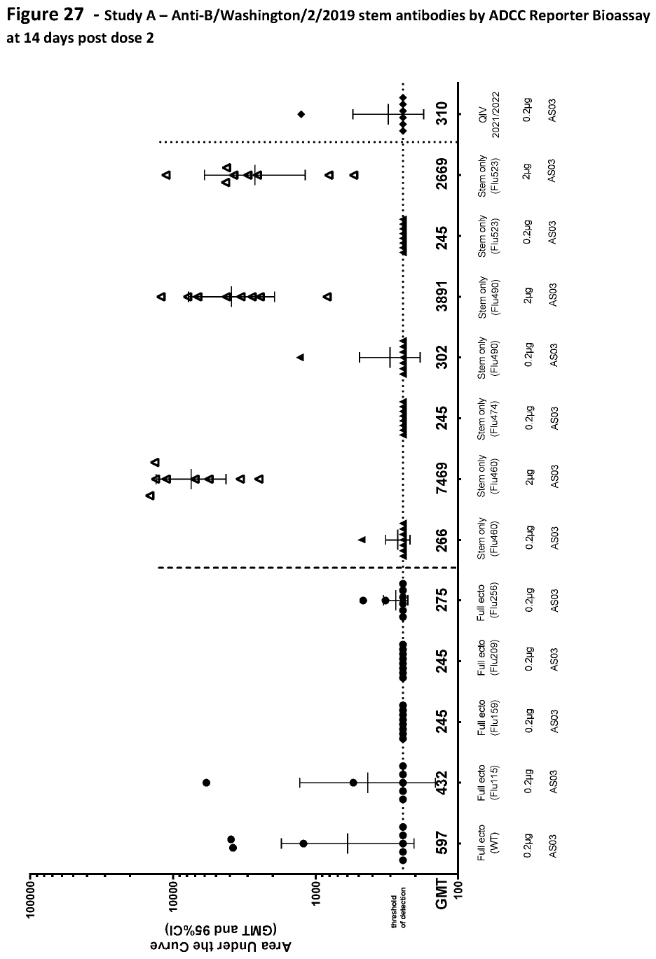

The full-length sequence of the HA polypeptide from B/Washington/02/2019 (Wash19) is used herein as a reference sequence. This sequence is shown in FIG. 2(a) and SEQ ID NO: 1. The specific amino acid sequences and locations referred to herein relate to this reference sequence. However it will be evident that for other B strain isolates and sequences where the numbering and/or amino acids at specific positions may differ, the equivalent sequences and locations in those other B strain isolates and sequences are also included within the scope of the polypeptide and polynucleotide constructs described herein.

In one embodiment, a suitable linker is included at or near to (e.g. within a few amino acids of) the position of a deleted sequence. Suitable linkers may be needed to allow correct folding of the HA stem antigen in the region of a deleted sequence. Typically a suitable linker will be flexible so as to allow such correct folding. Examples of suitable linkers for use in the HA stem antigen include but are not limited to G, GS, GSG, GSGS [SEQ ID NO: 53], GSPG [SEQ ID NO: 54], GPSG [SEQ ID NO: 55], GPSPG [SEQ ID NO: 56], GSGSG [SEQ ID NO: 57], GSGGSG [SEQ ID NO: 58], DVANKVSKATDGSG [SEQ ID NO: 59], DVANKVSKATDGSGG [SEQ ID NO: 60], DVANKVSKAGS [SEQ ID NO: 61], DVANKVSKAGG [SEQ ID NO: 62]. Further examples of linkers are as present in the batch 3 stem only selected constructs Flu452 to Flu536 as shown in FIG. 3(c) [SEQ ID NOS: 63-82].

Typically, linker 1 will be shorter than linker 2.

Typically, a linker at or near deletion 1 is an amino acid sequence of between 1-15 amino acids, such as for example a linker 1 shown in FIG. 5(a), 5(d), or 3(c), more particularly those present in the selected constructs shown in FIG. 3(a), 3(b) or 3(c).

Typically, a linker at or near deletion 2 is an amino acid sequence of between 3-25 amino acids, such as for example a linker 2 shown in FIG. 5(a), 5(d), or 3(c), more particularly those present in the selected constructs shown in FIG. 3(a), 3(b) or 3(c).

A stem antigen described herein typically comprises a linker 1 at or near deletion 1 and a linker 2 at or near deletion 2. Combinations of deletion 1 and 2 and linker 1 and 2 present in a stem antigen described herein may be as shown in the constructs in FIG. 5(a), 5(d), or 3(c), more particularly those present in the selected constructs shown in FIG. 3(a), 3(b) or 3(c).

In one embodiment, the stem antigen comprises a linker 1 in place of a deleted sequence of contiguous amino acids in HA1 as described hereinabove, wherein linker 1 is selected from G, GS, GSG or any of SEQ ID NOs: 63 to 76, in particular SEQ ID NOs: 63 to 76. In one embodiment, the stem antigen comprises a linker 2 in place of a deleted sequence of contiguous amino acids in HA2 as described hereinabove, wherein linker 2 is selected from GSG or any of SEQ ID NOs: 53 to 62 or 77 to 82, in particular any of SEQ ID NOs: 77 to 82.

Influenza B stem antigens described herein can be defined in terms of regions or domains of HA, in addition to or as well as in terms of sequences. A domain of a polypeptide or protein is a structurally defined element within the polypeptide or protein. Thus, in one embodiment, the stem antigen comprises influenza HA B strain haemagglutinin without a receptor binding domain. In a further embodiment, the stem antigen comprises HA B strain haemagglutinin without a receptor binding domain and without a vestigial esterase subdomain. See FIG. 7. However, it will be evident that there may be elements of the head domain sequences that remain in a stem construct, depending on the exact location of the deleted sequences.

In one embodiment, the influenza B stem antigens described herein are capable of binding to antibodies directed to at least one epitope on the wild type HA stem, suitably a neutralising epitope. Thus, modifications to the wild type influenza B HA in the antigens described herein preserve the conformation of at least one stem epitope. In one embodiment, the recombinant influenza B strain HA stem antigen retains one or more stem epitopes present in the wild type, suitably the CR9114 or the 5A7 epitope, or both the CR9114 and the 5A7 epitopes. In another embodiment the HA stem retains the CR9114 epitope but not the 5A7 epitope.

In another embodiment, the recombinant HA antigen comprises both an HA stem and an HA head domain. This is also referred to herein as an ectodomain or full ectodomain, meaning that both head and stem domains are present. In one embodiment, the one or more mutations in a full ectodomain HA antigen comprise one or more amino acid substitutions (e.g. point mutations) designed to stabilise the antigen.

Typically, the recombinant influenza B strain HA antigen described herein contains a number of amino acid substitutions (e.g. point mutations) such as up to 4 or up to 6 or up to 8 or up to 10 amino acid substitutions, e.g. between 1 and 4 or 1 and 6 amino acid substitutions, e.g. 1, 2, 3, 4, 5 or 6 amino acid substitutions compared to native HA. Typically the amino acid substitutions (e.g. point mutations) are in HA2. In the case of HA stem antigens, the recombinant HA antigen typically has a deletion of contiguous amino acids from HA1 and optionally a deletion of contiguous amino acids from HA2, wherein the deleted section is typically longer for HA1 than in HA2. There may be amino acid additions present in the recombinant HA, for example in place of deleted sections of contiguous amino acids. Thus, for HA stem antigens the recombinant HA may comprise an addition of a linker sequence to HA1 and optionally a linker sequence to HA2. In the case of HA ectodomain antigens, there are typically no deletions of contiguous amino acids from within HA1 or HA2 and typically no additions (insertions) of amino acids within HA1 or HA2.

In one embodiment, the recombinant HA stem or ectodomain antigen does not comprise additional elements of HA such as the transmembrane region or cytosolic region. In one embodiment, the HA antigen is not membrane bound, due to the absence of the transmembrane region, or the absence of both the transmembrane and cytosolic regions. In an alternative embodiment, the HA stem or ectodomain antigen comprises a transmembrane region such as an HA transmembrane region, with or without the cytosolic region. The transmembrane region of influenza B strain HA is located from around amino acid positions 551-568 and the cytosolic tail from around positions 568-582. See schematic diagram in FIG. 9.

### Prefusion Conformation

The influenza B strain HA antigen, whether stem or ectodomain, is suitably stabilised in the prefusion conformation or state. The influenza HA antigen may be synthetically stabilised (in the absence of certain regions of the wild type antigen). Stabilisation may be achieved for example by helix stabilization, loop optimization, disulphide bond addition, and side-chain repacking. The HA is in the form of a homotrimer which may be formed from monomers of HA stem without a head domain, or from monomers of an HA ectodomain comprising an HA stem domain and an HA head domain. Stabilisation may be achieved by mutations in the coiled coil region which stabilise the HA trimer. Alternatively or additionally stabilisation may be achieved by the presence of a trimerization domain. In one embodiment, the recombinant HA antigen comprises a heterologous trimerization domain. Mutations in the coiled coil region can potentially stabilise the HA once the trimerization domain has been removed following expression of the recombinant HA antigen, for example by enzymatic cleavage. Thus, in one embodiment, the HA antigen is expressed with a trimerization domain which is removed following expression. In a particular embodiment, the HA B strain antigen is an ectodomain antigen without a trimerization domain, more particularly an ectodomain antigen expressed with a trimerization domain and from which the trimerization domain has been cleaved following expression.

Stabilisation of HA may be achieved by introducing mutations (e.g. point mutations) that form or strengthen ionic bonds, salt bridges, or that increase hydrophobic packing or cavity filling.

Hydrophobic packing promotes and/or drives the association of hydrophobic regions together to exclude water. Cavity filling fills an unoccupied cavity volume found either buried inside (i.e. inside the monomer) or at the interface of (i.e. between the monomers in the trimer) the haemagglutinin protein by introduction of an amino acid (e.g. a point mutation) that fills such a space and allows for and/or promotes good folding/packing and avoids the tendency for water to become encapsulated or incorporated into the folding of the protein. This may be achieved for example by replacing an amino acid with a small side chain such as (but not limited to) serine, with an amino acid with a larger side chain. Stabilisation of homotrimeric HA antigen may be assessed by characterisation studies such as those described in the Examples. In one embodiment, stabilisation in prefusion form is assessed by determining the presence of trimeric HA. Alternatively or additionally, stabilisation in prefusion form may be assessed by determining the presence of epitopes such as the CR9114 and/or 5A7 epitopes, by means of a mAb binding assay.

The prefusion conformation of HA is discussed in the literature for example Wu & Wilson, Viruses, 2020, 12:1053, and Ni et al 2014. Stabilising mutations are discussed in the literature for example in Yassine et al, Nature Medicine, 2015, 21 (9): 1065-1070.

It will be understood that additional alterations may be present in both the head and stem regions compared to wild type HA that will either not negatively affect, or that will further optimise, the recombinant influenza B strain HA described herein. For example, amino acid insertions, deletions or substitutions may be made that do not disrupt the prefusion conformation of the HA or negatively impact the antigenic properties of the HA described herein. Such amino acid substitutions, deletions or insertions may be for example, for the purpose of altering or improving the properties of one or more epitopes of the HA either in the globular head or in the stem region, or both.

### Ectodomain

In one embodiment, the ectodomain comprises all or substantially all of the globular head. By substantially all of the globular head is meant at least 75% or at least 85% or at least 95% of the length of amino acid sequence present in a wild type influenza HA globular head.

In one embodiment the ectodomain comprises all or substantially all of the stem region. By substantially all of the stem region is meant at least 75% or at least 85% or at least 95% of the length of amino acid sequence present in a wild type influenza HA stem region.

In one embodiment the ectodomain comprises all or substantially all of the globular head and all or substantially all of the stem region, of a wild type influenza HA ectodomain.

The HA ectodomain is made up from two chains, HA1 and HA2. HA1 refers to the region of the HA protein including amino acid residues from approximately 1-359 of the HA protein. HA1 comprises all residues that are N-terminal to the HA1/HA2 cleavage peptide of the precursor HA0 protein, including the receptor binding domain of the HA protein.

HA2 refers to the region of the HA protein including amino acid residues from approximately 360-582 of the HA0 polypeptide. The HA2 chain comprises all residues that are after the HA1/HA2 cleavage peptide of the precursor HA0 protein, including the hydrophobic peptide responsible for insertion within the host cell membrane during the process of membrane fusion, transmembrane domain and cytosolic tail.

In one embodiment, the B strain HA ectodomain antigen therefore comprises amino acid residues 1-536 or an immunogenic fragment or derivative thereof having both a head and a stem domain.

### Stabilising Mutations

Stabilisation may be determined by looking at one or more different parameters following expression and purification, such as productivity of expression of the recombinant antigen relative to productivity of the wild type antigen when it is expressed recombinantly. Higher productivity is often associated with more stable folding of a recombinant protein. Alternatively or additionally, structural analyses may be performed such as nanoDSF and/or resistance to stress tests, to confirm improved folding stability. Stress tests can include evaluating the level of degradation and/or aggregation after a week at room temperature or 37° C. Alternatively or additionally, stabilisation may be assessed by presence of trimers and/or by examining presence of epitopes such as the CR9114 and the 5A7 epitopes. Suitably, a stabilised B strain HA antigen will be equal to or improved over wild type recombinant HA in relation to at least one parameter, suitably two or more parameters.

The influenza B strain HA trimeric antigen described herein may be stabilised by mutations introduced into the coiled coil region, suitably site-specific mutations for example individual amino acid substitutions, additions or deletions designed to confer improved stability on the HA antigen or trimer. These may be mutations in the coiled coil core or in the region immediately surrounding the coiled coil core. In one embodiment, the HA antigen comprises one or more stabilising mutations in a helix structure, for example helix A or helix B of prefusion influenza B strain HA, see FIG. 8. In another embodiment, the HA antigen comprises one or more stabilising mutations in one or more of Regions A, B and C (see FIG. 8). For example, it has been found that a mutation (e.g. a single point mutation) at one or more of positions 410, 429, 432, 450, 462, 465, 472 and 477 of HA from B Victoria strain B/Washington/02/2019, can help to stabilise the ectodomain trimer, and that a mutation (e.g., a single point mutation) at one or more of positions 410, 419, 446, 447, 449, 450, 451, 462, 465 and 477 can help to stabilise the stem antigen. In particular, it has been found that an amino acid substitution at position 465 (for hydrophobic reinforcement of the coiled coil) and/or 477 (introducing a non-charged residue in place of hydrophobic one for cavity filling/hydrogen bonding network creation) can have a beneficial effect on a recombinant stem or ectodomain influenza B strain HA. For the purposes herein, substitution refers to replacement of the wild type amino acid with any other amino acid at the same amino acid position.

In one embodiment, the influenza B strain HA antigen comprises a stabilising amino acid substitution (e.g., a single point mutation) at position 465 and/or 477.

In one embodiment, the influenza B strain HA antigen, in particular an ectodomain, comprises one or more, two or more, three or more, four or more, five or more, six or more, seven or more, or eight of the following mutations (e.g. point mutations) compared to wild type HA: N410M, M429P, L432P, T450V/L/I/M (such as T450V/M), S462V, G465V/L/I/M/F/W (such as G465V/L/F/I), E472V/L/I/M/F/W (such as I/L/V), and L477Q. Particular combinations of mutations (e.g. point mutations) that may be present in an influenza B strain HA ectodomain antigen according to the invention are shown in FIGS. 4 and 5.

In one embodiment, the influenza B strain HA antigen, in particular a stem antigen, comprises one or more, two or more, three or more, four or more, five or more, six or more, seven or more, or eight or more or nine or more or ten of the following mutations (e.g. point mutations) compared to wild type HA: N410M, V419S, L446S/Q/D (such as S/Q), R447L/A (such as A), T450V, D449K, 1451N, S462V, G465F/V/L, and L477Q. Particular combinations of mutations (e.g. point mutations) that may be present in an influenza B strain HA stem antigen according to the invention are shown in FIGS. 3 and 5.

The mutations described herein take their numbering from B/Washington/02/2019, the full length HA sequence of which is shown in SEQ ID NO: 1 and FIG. 2. This sequence includes the signal sequence, HA1, HA2, transmembrane and cytosolic regions. It will be evident that equivalent locations for substitutions and other mutations in HA ectodomains from influenza B strains other than B/Washington/02/2019, for example other B Victoria lineage strains, or B Yamagata lineage strains, are included within the present scope and can be located by reference to sequence comparisons with the B/Washington/02/2019 sequence presented herein.

In certain embodiments the antigen, which may be any influenza HA B strain antigen including a stem or an ectodomain antigen, in particular an ectodomain antigen, comprises a mutation or combination of mutations presented in Table 1 (a). Examples of particular HA antigens comprising the Table 1 mutations are described in FIGS. 3, 4 and 5 and in the Examples.

TABLE 1(a)

| | Position | | | | | | | |
|---|---|---|---|---|---|---|---|---|
| | 410 | 429 | 432 | 450 | 462 | 465 | 472 | 477 |
| | Wild type | | | | | | | |
| | N | M | L | T | S | G | E | L |
| Combinations | — | P | — | — | — | — | — | — |
| | — | — | P | — | — | — | — | — |
| | — | P | P | — | — | — | — | — |
| | M | — | — | — | — | — | — | — |
| | — | — | — | V | — | — | — | — |
| | — | — | — | M | — | — | — | — |
| | — | — | — | — | — | V | — | — |
| | — | — | — | — | — | L | — | — |
| | — | — | — | — | — | F | — | — |
| | — | — | — | — | — | — | I | — |
| | — | — | — | — | — | I | L | — |
| | — | — | — | — | — | — | — | Q |
| | — | — | — | V | — | L | — | — |
| | — | — | — | M | — | F | — | — |
| | M | — | — | M | V | — | — | — |
| | M | — | — | — | V | — | V | — |
| | M | — | — | M | V | — | V | — |
| | — | — | — | — | — | L | — | Q |
| | — | — | — | — | — | F | — | Q |
| | — | P | P | — | — | L | — | Q |
| | M | — | — | — | V | L | — | Q |
| | — | — | — | — | — | — | I | Q |
| | — | — | — | — | — | — | L | Q |
| | — | — | — | — | — | I | I | Q |
| | — | — | — | — | — | F | I | Q |
| | — | — | — | — | — | I | L | Q |
| | — | — | — | — | — | F | L | Q |
| | M | P | P | — | V | — | — | |
| | M | P | P | — | V | L | — | Q |

In certain embodiments the antigen, which may be any influenza HA B strain antigen, including a stem or an ectodomain antigen, in particular a stem antigen, comprises a mutation or combination of mutations presented in Table 1 (b). Examples of particular HA antigens comprising the Table 1 mutations are described in FIGS. 3, 4 and 5 and in the Examples.

TABLE 1(b)

| | Position | | | | | | | | | | | |
|---|---|---|---|---|---|---|---|---|---|---|---|---|
| | 410 | 419 | 429 | 432 | 446 | 447 | 450 | 451 | 462 | 465 | 472 | 477 |
| | Wild type | | | | | | | | | | | |
| | N | V | M | L | L | R | T | I | S | G | E | L |
| Combinations | M | — | — | — | — | — | — | — | V | — | — | — |
| | — | — | — | — | — | — | — | — | — | F | — | Q |
| | M | — | — | — | — | — | — | — | V | V | — | |
| | M | — | — | — | — | — | — | — | V | F | — | Q |
| | M | — | — | — | — | — | — | — | V | F | — | — |
| | — | — | — | — | S | — | V | — | — | — | — | — |
| | — | S | — | — | Q | — | V | N | — | — | — | — |
| | — | S | — | — | Q | — | V | N | — | L | — | — |
| | M | — | — | — | S | A | — | — | V | F | — | Q |

The mutations described in Tables 1 (a) and (b) target different regions or zones of HA, which are located by reference to the annotated amino acid sequence and HA structure diagram in FIG. 8:
i) Region A which is the membrane distal fusion domain region;
(ii) Region B which is the central fusion domain region; and
(iii) Region C which is the fusion peptide/membrane proximal fusion domain region.

In one embodiment, the influenza B strain HA antigen comprises one or more Region A mutations for example a substitution (e.g. a point mutation) at a position selected from one or more of 419, 429, 432, 446, 447, 449, 450 and 451.

In another embodiment, the antigen comprises one or more Region B mutations for example a substitution (e.g. a point mutation) at a position selected from one or more of 410, 462 and 465.

In another embodiment, the antigen comprises one or more Region C mutations for example a substitution (e.g. a point mutation) at a position selected from one or both of 472 or 477.

In further embodiments, the antigen comprises one or more mutations from a combination of each of Region A and B, or each of Region A and C, or each of Region B and C, or all three regions A, B and C. In a particular embodiment, the antigen comprises one or more mutations from Region B and one or more mutations from Region C, for example a substitution at position 465 and a substitution at position 472 and/or 477. In a particular embodiment, the antigen comprises a substitution (e.g. a point mutation) in Region B at position 465 for hydrophobic reinforcement at the coiled coil interface such as G465F/L in particular G465F, and/or a substitution (e.g. a point mutation) in Region C at position 477 which replaces a hydrophobic residue with a non-charged residue for cavity filling/hydrogen bond network creation, in particular L477Q.

In a particular embodiment, an influenza B strain ectodomain antigen as described herein has a combination of mutations selected from the combinations shown in Table 1 (a).

In another embodiment, an influenza B strain stem antigen as described herein has a combination of mutations selected from the combinations shown in Table 1 (b), for example a combination of mutations at locations 410, 462, 465 and 477 or 410, 462, 465, 477, 446 and 447, such as N410M, S462V, G465F and L477Q, or N410M, S462V, G465F, L477Q, L446S and R447A.

In one embodiment, the mutations (e.g. point mutations) introduced into the stem or ectodomain HA described herein do not create disulphide bonds.

The influenza B strain HA antigen may be comprised within a construct which comprises further polypeptide sequences. The further polypeptide sequences may include, for example, one or more signal peptides. In some embodiments, the signal peptide is not present in the final construct of the HA antigen or compositions or methods or uses herein.

In particular embodiments, the HA antigen comprises or more suitably consists of a construct described in FIG. 3 or FIG. 4, such as a polypeptide sequence selected from SEQ ID NOS: 2-25 and 84-121 with or without the signal peptide, or a sequence having at least 85% identity or at least 87% identity or at least 90% identity, such as 95% or greater, such as 98% or greater, such as 99% or greater sequence identity to any one of the amino acid sequences of SEQ ID NOS: 2-25 and 84-121 with or without the signal peptide. In further embodiments, the HA antigen comprises a polypeptide sequence of SEQ ID NOS: 2-25 or 84-121, with or without the signal peptide, from which certain elements, such as the His tag or the His tag and the trimerization domain, are absent. In particular embodiments, the HA antigen comprises a polypeptide sequence of SEQ ID NOS: 2-25 and 84-121, with or without the signal peptide, or a sequence having at least 85% identity or at least 87% identity or at least 90% identity, such as 95% or greater, such as 98% or greater, such as 99% or greater sequence identity to any one of the amino acid sequences of SEQ ID NOS: 2-25 and 84-121, with or without the signal peptide, and without the trimerisation domain and without the His tag and further comprising a transmembrane domain (which may be homologous or heterologous and is optionally trimeric), and cytosolic domain. For example, the HA antigen may comprise one of the following, each of which may be without the signal peptide in its final form:
(1) A stem antigen comprising deletions, mutations and linkers as shown for Flu075 in FIG. 3, suitably SEQ ID NO: 6, with or without a trimerisation domain and His tag;
(2) A stem antigen comprising deletions, mutations and linkers as shown for Flu077 in FIG. 3, suitably SEQ ID NO: 7, with or without a trimerisation domain and His tag;
(3) An ectodomain antigen comprising mutations as shown for Flu115 in FIG. 4, suitably SEQ ID NO: 17, with or without a trimerisation domain and His tag;
(4) An ectodomain antigen comprising mutations as shown for Flu159 in FIG. 4, suitably SEQ ID NO: 20, with or without a trimerisation domain and His tag.
(5) A stem antigen comprising deletions, mutations and linkers as shown for Flu460 in FIG. 3, suitably SEQ ID NO: 109, with or without a trimerization domain and His tag;
(6) A stem antigen comprising deletions, mutations and linkers as shown for Flu474 in FIG. 3, suitably SEQ ID NO: 111, with or without a trimerization domain and His tag;
(7) A stem antigen comprising deletions, mutations and linkers as shown for Flu490 in FIG. 3, suitably SEQ ID NO: 113, with or without a trimerization domain and His tag;
(8) A stem antigen comprising deletions, mutations and linkers as shown for Flu523 in FIG. 3, suitably SEQ ID NO: 118, with or without a trimerization domain and His tag;
(9) An ectodomain antigen comprising mutations as shown for Flu209 in FIG. 4, suitably SEQ ID NO: 84, with or without a trimerisation domain and His tag.
(10) An ectodomain antigen comprising mutations as shown for Flu256 in FIG. 4, suitably SEQ ID NO: 91, with or without a trimerisation domain and His tag.

In further embodiments, the HA antigen comprises a polypeptide sequence selected from (1) to (10) above, with or without the signal peptide, and without the trimerisation domain or the His tag, and further comprising a transmembrane domain, which may be homologous or heterologous and is optionally trimeric, and optionally a cytosolic domain. The transmembrane domain may be an HA transmembrane domain, such as the transmembrane domain from the influenza strain from which the antigen is derived i.e. a homologous transmembrane domain. The cytosolic domain may be an

HA cytosolic domain, such as the cytosolic domain from the influenza strain from which the antigen is derived. The purpose of the transmembrane and cytosolic domains is to function as a membrane anchor for the HA antigen. In further embodiments, the HA antigen comprises a polypeptide sequence selected from (1) to (10) above, with or without the signal peptide, and without the trimerization domain or the His tag, and further comprising ferritin such as H. pylori ferritin. Particular embodiments are given in SEQ ID NOs: 160, 162, 164 and 166 which are membrane anchored i.e. fused to a membrane anchoring region, and SEQ ID NOs: 161, 163, 165 or 167 which are ferritin fused. Further embodiments are given in SEQ ID NOs: 168, 170, 172, 174, 176 and 178 which are fused to a membrane anchoring region and SEQ ID NOs: 169, 171, 173, 175, 177 and 179 which are fused to ferritin. Such antigens are also contemplated for nucleic acid vaccine delivery.

Particles/Nanoparticles e.g. Ferritin Nanoparticles

The influenza B strain HA antigen described herein may be presented on the surface of nanoparticles, in a strategy known as nano particularization. In one embodiment, the influenza B strain HA stem or ectodomain antigen is presented on the surface of self-assembling protein nanoparticles, suitably ferritin nanoparticles, such as more suitably insect or bacterial ferritin nanoparticles, such as most suitably H. pylori ferritin nanoparticles (such as those disclosed in Corbett mBio. 2019 10 (1): e02810-18, WO2013/044203, WO2015/183969 and WO2018/045308). It will be evident that alternative protein nanoparticles known in the art may also be used such as but not limited to lumazine and encapsulin, or other protein nanoparticles including artificially constructed protein nanoparticles.

In a particular embodiment, the influenza B strain HA stem or ectodomain is fused to a heterologous polypeptide, such as ferritin. When ferritin is expressed in a fusion with an HA antigen, it can act as a trimerisation domain for the formation and/or stabilisation of HA trimers. Suitably, the heterologous polypeptide (such as ferritin) and the influenza HA stem or ectodomain monomer are connected by a linker, such as for example a linker comprising the polypeptide sequence SGG, such as consisting of the polypeptide sequence SGG, although it will be understood that longer linkers can also be used and linker design can be further adjusted.

Particularization technologies are well known and fusion strategies such as ferritin fusion described here are just one example. Other suitable examples include conjugation, which can be achieved chemically or by using other approaches to protein ligation such as the Streptococcus pyogenes derived system known as SpyTag/SpyCatcher. Multi-component nano particularization technologies can also be applied in which more than one, such as two or more different influenza antigens are displayed. Examples include a fusion with a heterologous polypeptide such as insect ferritin, or combining different antigens in a nanoparticle by means of chemical conjugation. Insect ferritin can be engineered to display two different trimeric antigens in a defined ratio and geometric pattern.

In one embodiment, an influenza HA B strain stem or ectodomain antigen described herein, expressed in a fusion with a transmembrane domain (HA-TM), may be extracted and formulated to generate micelle structures with the transmembrane domain inside and the HA stem or ectodomain protruding outside the structure. In another embodiment, extracted HA-TM, is introduced into a liposomal membrane (e.g. AS01 liposomes).

In a further embodiment, the influenza B strain HA stem or ectodomain is in the form of rosette structures such as those described in WO2017/149054. In a particular embodiment, the influenza B strain HA stem or ectodomain is fused to a hydrophobic signal such as a transmembrane domain (which may be a homologous or a heterologous transmembrane domain) and a heterologous trimerization domain, which constructs form rosette structures in vivo or in vitro.

### Immunogenic Fragments

The influenza HA stem and ectodomain antigens described herein also encompass immunogenic fragments of the HA stem and ectodomain regions which fragments comprise the mutations described herein. In one embodiment the influenza HA stem or ectodomain is a polypeptide consisting of an influenza HA ectodomain antigen described herein, or an immunogenic fragment thereof containing the mutations (e.g. point mutations) described herein. In another embodiment, the influenza HA stem or ectodomain is a polypeptide consisting of an influenza HA stem antigen described herein, or an immunogenic fragment thereof containing the mutations (e.g. point mutations) described herein.

The immunogenic fragment of an influenza HA stem or ectodomain of use in the present invention comprises, such as consists of, a fragment of an influenza HA stem or ectodomain which is capable of eliciting neutralising antibodies and/or a T cell response (such as a CD4 or CD8 T cell response) to influenza B virus, suitably a protective immune response (e.g. reducing partially or completely the severity of one or more symptoms and/or time over which one or more symptoms are experienced by a subject following infection, reducing the likelihood of developing an established infection after challenge and/or slowing progression of illness (e.g. extending survival)).

Suitably the immunogenic fragment of an influenza HA stem or ectodomain comprises one or more epitopes from a full length influenza HA stem or ectodomain, such as one, two or three or more epitopes.

### HA Epitopes

Certain epitopes on influenza B strain HA are known to be neutralising epitopes for influenza virus B strain. These include the CR9114 stem epitope, the 5A7 stem epitope, the CR8033 epitope and the CR071 epitope (CR9114, CR8033 and CR071 are described for example in Dreyfus et al Science, 2012, 337 (6100): 1343-8; and 5A7 in Yasugi et al PLOS Pathogens, 2013, 9 (2): e1003150).

In one embodiment, the CR9114 epitope is present in the influenza B strain HA antigen described herein. In another embodiment, the 5A7 epitope is present in the influenza B strain HA antigen described herein. In another embodiment, one or two or three or all four epitopes selected from CR9114, 5A7, CR8033 and CR8071 epitopes are present. In another embodiment, both the CR9114 and 5A7 epitopes are present in the HA antigen. When epitopes are present in the recombinant HA antigen, this means that mAbs directed against those epitopes are capable of binding to the recombinant antigen in a suitable assay.

### Recombinant HA Antigen

A recombinant HA antigen comprises or is encoded by one or more nucleic acids that are derived from a nucleic acid which was artificially constructed. For example, the nucleic acid can comprise, or be encoded by, a cloned nucleic acid formed by joining heterologous nucleic acids.

The recombinant HA antigen includes haemagglutinin-derived sequences HA1 and HA2 of the ectodomain, or part of those sequences in the case of stem only antigen, and may include other non-haemagglutinin derived sequences, for example a linker sequence which may be a flexible linker sequence, or a cleavage site such as a furin cleavage site. A linker sequence may be present for example between the HA1 and HA2 regions of the recombinant HA. A linker sequence can facilitate the independent folding of the HA domains. The linker sequence may be an amino acid sequence that is synthesized as part of a recombinant fusion protein. In other embodiments a chemical linker is used to connect synthetically or recombinantly produced sub-sequences. Such flexible linkers are known to those skilled in the art. Alternatively a cleavage site, such as a furin cleavage site, may be present between the HA1 and HA2 regions of the HA. A furin cleavage site can be used to allow activation e.g. from a vector technology. This may improve antigen representativity and immunogenicity.

Linkers may alternatively or additionally be present at or near a region of deleted HA1 or HA2 or both, as already discussed.

The recombinant HA antigen may further comprise, in addition to or as an alternative to flexible linkers, polypeptide sub-sequences from proteins which are unrelated to haemagglutinin, for example a sequence with affinity to a known antibody to facilitate affinity purification and/or detection. Such detection and purification-facilitating domains include, but are not limited to, metal chelating peptides such as polyhistidine tracts and histidine-tryptophan modules that allow purification on immobilized metals and protein A domains that allow purification on immobilized immunoglobulin. Examples include heterologous fusion sequences encoding gD tags, AviTag, c-Myc eptiopes, polyhistidine tags, fluorescent proteins (e.g. GFP), beta-galactosidase protein or glutathione S transferase or any other sequence useful for detection or purification of the fusion protein expressed in or on a cell. A preferred further polypeptide sequence is a polyhistidine tag, such as a four or a six or an eight or a ten-histidine tag, in particular a six-histidine tag. The inclusion of a cleavable linker sequence between the purification domain (e.g. polyhistidine tag) and the HA antigen may be useful to facilitate purification. For example, an enzyme cleavage site, such as a TEV cleavage site or a thrombin cleavage site, may be included between the further polypeptide and the rest of the recombinant HA sequence.

A cleavable linker sequence, for example an enzyme cleavage site such as a TEV cleavage site or a thrombin cleavage site may alternatively or additionally be included between the trimerization domain and the rest of the recombinant HA sequence. This can allow the trimerization domain to be removed in the final recombinant HA. Hence, the recombinant HA described herein may comprise or consist of (in order) a stem or ectodomain of HA, heterologous trimerization domain, purification tag (e.g. polyhistidine tag) and optionally a cleavable linker sequence i) between the purification tag and the rest of the recombinant HA and/or ii) between the trimerization domain and the HA stem or ectodomain.

In some embodiments, the His tag and optionally the trimerization domain (e.g. foldon or GCN4) if present, are cleaved following expression of the protein and thus are not present in the final HA antigen.

For example, the recombinant HA antigen described herein may comprise i) an amino acid sequence comprising the stem or ectodomain of HA, and ii) a heterologous trimerization domain e.g. foldon as shown in SEQ ID NO: 26 or a derivative thereof that maintains the ability to induce recombinant HA monomers to form trimers. In particular, the recombinant HA antigen described herein may comprise or consist of any one of the amino acid sequences shown in SEQ ID NOS: 2 to 25 and 84 to 121. These sequences all contain a foldon, which is optionally removed to provide the final HA antigen.

A recombinant HA antigen described herein presented in a nanoparticle may comprise for example i) an amino acid sequence comprising the stem or ectodomain of HA, such as any of the amino acid sequences shown in SEQ ID NOS: 2 to 35 and 84 to 121, minus the foldon; and optionally ii) a heterologous polypeptide capable of forming a nanoparticle such as ferritin. In one embodiment the recombinant HA antigen is a stem or ectodomain described herein, fused to H. pylori ferritin, for example as shown in SEQ ID Nos: 161, 163, 165, 167, 169, 171, 173, 175, 177 and 179.

A recombinant HA antigen described herein delivered as nucleic acid such as mRNA, may comprise i) an amino acid sequence comprising the stem or ectodomain of HA, and optionally ii) a transmembrane domain (homologous or heterologous, and optionally trimeric) such that the antigen is membrane anchored once the antigen is expressed, or a heterologous polypeptide capable of forming a nanoparticle, such as ferritin, such that the antigen once expressed is presented on the surface of nanoparticles.

### Polynucleotide Encoding HA Antigen

A polynucleotide construct encoding the recombinant HA antigen may include a signal sequence. Typically, the signal sequence is appropriate for the host cell in which the recombinant HA is expressed. In one embodiment, the wild type signal peptide sequence is used. In another embodiment, a heterologous signal peptide sequence is used.

Accordingly, a polynucleotide encoding the recombinant influenza B strain HA antigen described herein may comprise a sequence encoding stem or ectodomain of HA, a heterologous trimerization domain (e.g. foldon), a purification tag (e.g. polyhistidine tag) and a signal peptide (e.g. SEQ ID NO: 27), such as in the order: a signal peptide (e.g. SEQ ID NO: 27), stem or ectodomain of HA B strain, heterologous trimerization domain (e.g. foldon), purification tag (e.g. polyhistidine tag). In another example, the polynucleotide encoding the recombinant HA antigen described herein comprises a sequence encoding (in order): a signal peptide (e.g. SEQ ID NO: 27), the stem or ectodomain of HA, a cleavable linker sequence (e.g. TEV cleavage site), a heterologous trimerization domain (e.g. foldon), and a purification tag (e.g. polyhistidine tag).

The polynucleotide sequence encoding the recombinant influenza B strain HA antigen may comprise i) a polynucleotide sequence encoding the stem or ectodomain of influenza B strain HA and ii) SEQ ID NO: 26 encoding the foldon or a derivative of the foldon that maintains the ability to induce expressed recombinant HA monomers to form trimers.

In particular embodiments, the polynucleotide sequence encoding the recombinant HA antigen described herein comprises or consists of a sequence encoding the polypeptide of any one of SEQ ID NOs: 2 to 25 or 84 to 121, such as for example a polynucleotide sequence of SEQ ID NOs: 29 to 52 or 122 to 159.

In another embodiment, the polynucleotide sequence encoding the recombinant influenza B strain HA antigen comprises a sequence encoding the stem or ectodomain, and a transmembrane domain, such as for example any of SEQ ID NOs: 160, 162, 164, 166, 168, 170, 172, 174, 176 and 178. In one embodiment the transmembrane domain is the native influenza B strain HA transmembrane such as

SEQ ID NO: 28 or a functional derivative that anchors the antigen in a membrane. In one embodiment, the polynucleotide sequence is formulated for delivery as a nucleic acid vaccine.

In another embodiment, the polynucleotide sequence encoding the recombinant influenza B strain HA antigen comprises a sequence encoding the stem or ectodomain, and a polypeptide capable of forming a nanoparticle, such as for example any of SEQ ID NOs: 161, 163, 165, 167, 169, 171, 173, 175, 177 and 179. In one embodiment the polypeptide capable of forming a nanoparticle is ferritin. In one embodiment, the polynucleotide sequence is formulated for delivery as a nucleic acid vaccine.

Nucleic acid-based vaccines are contemplated herein for any of the influenza B strain HA antigens described. The nucleic acid may, for example, be RNA (i.e. an RNA-based vaccine or mRNA delivery platform) or DNA (i.e. a DNA-based vaccine, such as a plasmid DNA vaccine), including viral vectors. The sequence of the nucleic acid molecule may be modified, e.g. to increase the efficacy of expression or replication of the nucleic acid, or to provide additional stability or resistance to degradation, or to reduce reactogenicity or to activate the interferon pathway impacting antigen expression.

Messenger RNA (mRNA) can direct the cellular machinery of a subject to produce proteins. The term mRNA as used herein includes conventional mRNA or mRNA analogs, such as those containing modified backbones or modified bases (e.g. pseudouridine, or the like). mRNA, may or may not have a 5′ cap. The mRNA may encode more than one antigen. For example, the mRNA encoding an HA antigen as described herein may encode only the HA antigen or it may encode a second HA antigen or additional proteins. Where additional proteins are encoded, mRNA may be polycistronic.

mRNA may be non-replicating or may be replicating, also known as self-amplifying. A self-amplifying mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest.

mRNA may also be codon optimised. In some embodiments, mRNA may be codon optimised for expression in human cells.

A range of carrier systems have been described which encapsulate or complex mRNA in order to facilitate mRNA delivery and consequent expression of encoded antigens as compared to mRNA which is not encapsulated or complexed. The present invention may utilise any suitable carrier system. Particular carrier systems include lipid nanoparticles (LNPs) which are non-virion liposome particles in which mRNA can be encapsulated; cationic nanoemulsion (CNE) delivery systems in which cationic oil-in-water emulsions can be used to deliver the mRNA to the interior of a cell; and lipidoid-coated iron oxide nanoparticles (LION) which are capable of delivering mRNA into cells and may be aided after administration to a subject by application of an external magnetic field. In one embodiment the mRNA encoding an HA antigen as described herein is encapsulated or complexed in a carrier system selected from LNPs, CNE and LION.

### Trimerisation Domains

A suitable trimerization domain is one that induces the recombinant HA antigen monomers to form trimers. Suitably the trimerization domain is or is derived from the natural trimerization domain of the T4 phage fibritin “foldon”. A foldon sequence may be used which forms a B-propeller structure comprising the C terminus of the fibritin domain of the T4 bacteriophage. For example, the trimerization domain may comprise or consist of the foldon amino acid sequence shown in SEQ ID NO: 26 or a derivative of this sequence that maintains the ability to induce recombinant monomers to form trimers.

Another suitable trimerisation domain is a leucine zipper trimerization motif derived from the yeast transcription activator GCN4. Further suitable trimerization domains include chloramphenicol acetyl transferase (CAT). The trimerization domain is placed at the C terminus of the HA ectodomain, i.e. at the stem end of the HA. Further trimerization domains include a human-derived trimerization domain such as Trimer-Tag, or an HIV-derived trimerization domain. Typically, the trimerization domain is fused via a short linker region to the HA sequence. The region between the trimerization domain and the HA sequence may include a cleavable linker sequence, so it is possible to isolate the

HA sequence from the trimerization domain at a later stage. Thus, the HA sequence may be linked (e.g. in order), optionally via a linker sequence, to a heterologous sequence comprising a protease cleavage site, the trimerization domain and a purification tag such as a histidine tag to aid in purification. Such heterologous trimerization domains may be linked to HA sequences by techniques known in the art, such as molecular cloning.

### Preparation of Recombinant HA Antigen

Use of recombinant DNA technology to produce influenza vaccines offers several advantages. These include avoiding the steps of adaptation and passage of infectious viruses in eggs, and production of more highly purified protein under safer and more stringently controlled conditions. Moreover, no virus inactivation step has to be included. Any suitable cloning and expression system may be used to recombinantly produce the recombinant HA antigen.

Nucleotide sequences encoding the recombinant HA antigens of the invention may be synthesized, and/or cloned and expressed according to techniques well known to those in the art. See for example, Sambrook, et al. Molecular Cloning, A Laboratory Manual, Vols. 1-3, Cold Spring Harbor Press, Cold Spring Harbor, NY (1989). In some embodiments, the polynucleotide sequences will be codon optimised for a particular recipient host cell using standard methodologies. For example, a DNA construct encoding a haemagglutinin sequence can be codon optimised for expression in other hosts e.g. bacteria, mammalian or insect cells. Suitable host cells may include bacterial cells such as E. Coli, fungal cells such as yeast, insect cells such as Drosophila S2, Spodoptera Sf9, Sf00+ or Hi-5 and animal cells such as CHO.

Haemagglutinin sequences may be produced by standard recombinant methods known in the art, such as polymerase chain reaction (PCR) or reverse transcriptase PCR, reverse engineering, or the DNA can be synthesized. For PCR, primers can be prepared using haemagglutinin nucleotide sequences that are available in public databases.

### Sequence Identity

Identity with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA or MUSCLE or CLUSTALO, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 (a standard scoring matrix; see Dayhoff et al (1978) A model of evolutionary change in proteins, in Atlas of Protein Sequence and Structure, vol. 5, supp. 3) can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences (e.g., divided by the length of the alignment).

### Influenza Strains

The HA stem or ectodomain sequence of the recombinant influenza B strain HA antigens described herein may be derived from any influenza B virus strain, including strains from the two known lineages B (Victoria) and B (Yamagata).

In one embodiment, the HA sequence is from naturally occurring HA from a circulating influenza B virus or a strain recommended by the WHO for seasonal flu vaccines. For example, the circulating or vaccine-recommended influenza B virus may be a strain identified by WHO as a circulating or vaccine-recommended seasonal influenza virus strain, or identified by the WHO in a previous season as a circulating or vaccine-recommended seasonal strain. In one embodiment, the HA sequence is from a strain identified by the WHO as having the potential to cause an epidemic in a subsequent influenza season. In one embodiment, the HA sequence is from a strain which is a new influenza virus strain against which the large majority of the human population has no immunity. Typically, the WHO identifies and publicises such strains.

Currently circulating/WHO recommended B strains for flu vaccines are:
Yamagata strain: B/Phuket/3073/2013 (Phu13) and
Victoria strain: B/Washington/02/2019 (Wash19) (replaced in 2022 with Victoria strain: B/Austria/1359417/2021)

### Additional Antigens

The present invention may involve a plurality of antigenic components, for example with the objective to elicit a broad immune response to influenza virus. Consequently, more than one antigen may be present, more than one polynucleotide encoding an antigen may be present, one polynucleotide encoding more than one antigen may be present or a mixture of antigen(s) and polynucleotide(s) encoding antigen(s) may be present. Polysaccharides such as polysaccharide conjugates may also be present.

By the term antigen is meant a polypeptide which is capable of eliciting an immune response. Suitably the antigen comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

### Vaccine Delivery

It will be evident that the recombinant HA antigen described herein can be delivered in any suitable vaccine delivery mode, such as in the form of a protein, or in the form of nucleic acid, including nucleic acid delivery platforms such as DNA (including for example a viral delivery platform such as adenovirus) or RNA including for example mRNA, optionally formulated in a carrier system. Suitable delivery systems include viral vectors such as adenovirus vectors. In any case, the delivery mode will include a pharmaceutically acceptable diluent or carrier. Optionally one or more adjuvants may be used.

### Immunogenic Composition

In a further aspect, an immunogenic composition comprising an influenza B strain HA antigen or polynucleotide as described herein and a pharmaceutically acceptable carrier is provided.

In one embodiment, the immunogenic composition comprising a HA antigen as described herein further comprises an adjuvant. Preferably, the adjuvant is an oil-in-water emulsion adjuvant. Oil in water emulsion adjuvants are well known in the art and are described below in more detail.

In one embodiment, the immunogenic composition comprising a HA polynucleotide encoding a HA antigen as described herein further comprises a polynucleotide carrier or delivery system.

In one embodiment, the immunogenic composition is monovalent, i.e. comprises influenza HA antigen from only one B strain. In alternative embodiments, the composition is multivalent, i.e. comprises influenza virus antigens from multiple strains. For example, the composition may be bivalent, trivalent or quadrivalent, e.g. may contain two or three seasonal strains with the recombinant HA antigen described here.

In one embodiment, the immunogenic composition is an improved seasonal influenza vaccine in which the B strain HA antigen is capable of inducing an immune response against at least one other influenza B strain, from the same or a different lineage. In further embodiments, the immunogenic composition is capable of inducing an immune response against two or three or four or more different B strains including one or more from each of two different lineages such as B/Victoria and B/Yamagata.

In one embodiment there is provided an immunogenic composition comprising (i) an influenza B strain HA stem or ectodomain antigen in trimeric form, said antigen comprising one or more stabilising mutations described herein in the coiled coil region; and (ii) a squalene-based adjuvant.

### Adjuvant

In one embodiment, an immunogenic composition of the invention comprises an adjuvant. In particular, the adjuvant may be an emulsion, such as an oil-in-water emulsion. Optionally, other immunostimulants may be present in the oil-in-water emulsion. In a specific embodiment, an oil-in-water emulsion comprises a metabolisable, non-toxic oil such as squalene or squalane, optionally a tocol such as tocopherol in particular alpha tocopherol, and an emulsifier (or surfactant) such as the non-ionic surfactant polyoxyethylene sorbitan monooleate (TWEEN-80™ or polysorbate 80™). Mixtures of surfactants can be used such as polyoxyethylene sorbitan monooleate/sorbitan trioleate (SPAN 85™) mixtures, or polyoxyethylene sorbitan monooleate/t-octylphenoxypolyethoxyethanol (TRITON X-100™) mixtures.

In one aspect, the oil-in-water emulsion has one of the following compositions:
From 0.5 to 11 mg squalene, from 0.05 to 5% polyoxythylene sorbitan monooleate (TWEEN-80™ or POLYSORBATE 80™) and optionally, from 2 to 12% alpha-tocopherol; or
About 5% squalene, about 0.5% polyoxyethylene sorbitan monooleate (TWEEN-80™ or POLYSORBATE 80TM) and about 0.5% sorbitan trioleate (SPAN 85™). This adjuvant is called MF59.

Squalene emulsion adjuvants are described in more detail below.

An alternative adjuvant that may be used comprises an immunologically active saponin fraction derived from the bark of Quillaja Saponaria Molina (e.g. QS21) presented in the form of a liposome and a lipopolysaccharide (e.g. 3D-MPL), optionally further including a sterol (cholesterol). In one embodiment, the adjuvant comprises or consists of a saponin (e.g. QS21) presented in the form of a liposome, a lipid A derivative such as 3D-MPL and a sterol (e.g. cholesterol). The liposomes suitably contain a neutral lipid, for example, phosphatidylcholine, dioleoyl phosphatidylcholine (DOPC) or dilauryl phosphatidylcholine. The liposomes may also contain a charged lipid which increases the stability of the liposome-QS21 structure for liposomes composed of saturated lipids. An example of such an adjuvant is AS01, which comprises 3D-MPL and QS21 in a quenched form with cholesterol, and can be made as described in WO96/33739. Either the AS01B or AS01E forms of this adjuvant may be used. The AS01 B adjuvant comprises liposomes, which in turn comprise dioleoyl phosphatidylcholine (DOPC), cholesterol and 3D-MPL (in an amount of approximately 1000 micrograms DOPC, 250 micrograms cholesterol and 50 micrograms 3D-MPL per vaccine dose), QS21 (50 micrograms/dose), phosphate NaCl buffer and water to a volume of 0.5 ml.

The AS01E adjuvant comprises the same ingredients than AS01B but at a lower concentration in an amount of approximately 500 micrograms DOPC, 125 micrograms cholesterol, 25 micrograms 3D-MPL and 25 micrograms QS21, phosphate NaCl buffer and water to a volume of 0.5 ml.

In one embodiment, the influenza B strain HA stem or ectodomain antigen is in physical association with an adjuvant e.g. liposomes of AS01 or emulsion of a squalene-containing adjuvant such as AS03.

### Squalene Emulsion Adjuvant

The term ‘squalene emulsion adjuvant’ as used herein refers to a squalene containing oil-in-water emulsion adjuvant.

Squalene, is a branched, unsaturated terpenoid ([(CH3)2C[═CHCH2CH2C(CH3)]2=CHCH2-]2; C30H50; 2,6,10,15,19,23-hexamethyl-2,6,10,14,18,22-tetracosahexaene; CAS Registry Number 7683-64-9). Squalene is readily available from commercial sources or may be obtained by methods known in the art. Squalene shows good biocompatibility and is readily metabolised.

The squalene emulsion adjuvant may comprise one or more tocopherols, suitably wherein the weight ratio of squalene to tocopherol is 20 or less (i.e. 20 weight units of squalene or less per weight unit of tocopherol or, alternatively phrased, at least 1 weight unit of tocopherol per 20 weight units of squalene).

Any of the a, B, y, 8, E and/or & tocopherols can be used, but a-tocopherol (also referred to herein as alpha-tocopherol) is typically used. D-alpha-tocopherol and D/L-alpha-tocopherol can both be used. Tocopherols are readily available from commercial sources or may be obtained by methods known in the art. In some embodiments the squalene emulsion adjuvant contains alpha-tocopherol, especially D/L-alpha-tocopherol.

Squalene emulsion adjuvants will typically have a submicron droplet size. Droplet sizes below 200 nm are beneficial in that they can facilitate sterilisation by filtration. There is evidence that droplet sizes in the 80 to 200 nm range are of particular interest for potency, manufacturing consistency and stability reasons (Klucker, 2012; Shah, 2014; Shah, 2015; Shah, 2019). Suitably the squalene emulsion adjuvant has an average droplet size of less than 1 um, especially less than 500 nm and in particular less than 200 nm. Suitably the squalene emulsion adjuvant has an average droplet size of at least 50 nm, especially at least 80 nm, in particular at least 100 nm, such as at least 120 nm. The squalene emulsion adjuvant may have an average droplet size of 50 to 200 nm, such as 80 to 200 nm, especially 120 to 180 nm, in particular 140 to 180 nm, such as about 160 nm.

Uniformity of droplet sizes is desirable. A polydispersity index (PdI) of greater than 0.7 indicates that the sample has a very broad size distribution and a reported value of 0 means that size variation is absent, although values smaller than 0.05 are rarely seen. Suitably the squalene emulsion adjuvant has a polydispersity of 0.5 or less, especially 0.3 or less, such as 0.2 or less.

The droplet size, as used herein, means the average diameter of oil droplets in an emulsion and can be determined in various ways e.g. using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan). See Light Scattering from Polymer Solutions and Nanoparticle Dispersions Schartl, 2007. Dynamic light scattering (DLS) is the preferred method by which droplet size is determined. The preferred method for defining the average droplet diameter is a Z-average i.e. the intensity-weighted mean hydrodynamic size of the ensemble collection of droplets measured by DLS. The Z-average is derived from cumulants analysis of the measured correlation curve, wherein a single particle size (droplet diameter) is assumed and a single exponential fit is applied to the autocorrelation function. Thus, references herein to average droplet size should be taken as an intensity-weighted average, and ideally the Z-average. PdI values are easily provided by the same instrumentation which measures average diameter.

In order to maintain a stable submicron emulsion, one or more emulsifying agents (i.e. surfactants) are generally required. Surfactants can be classified by their ‘HLB’ (Griffin's hydrophile/lipophile balance), where a HLB in the range 1-10 generally means that the surfactant is more soluble in oil than in water, whereas a HLB in the range 10-20 means that the surfactant is more soluble in water than in oil. HLB values are readily available for many surfactants of interest or can be determined experimentally, e.g. polysorbate 80 has a HLB of 15.0 and TPGS has a HLB of 13 to 13.2. Sorbitan trioleate has a HLB of 1.8. When two or more surfactants are blended, the resulting HLB of the blend is typically calculated by the weighted average e.g. a 70/30 wt % mixture of polysorbate 80 and TPGS has a HLB of (15.0×0.70)+(13×0.30) i.e. 14.4. A 70/30 wt % mixture of polysorbate 80 and sorbitan trioleate has a HLB of (15.0×0.70)+(1.8×0.30) i.e. 11.04.

Surfactant(s) will typically be metabolisable (biodegradable) and biocompatible, being suitable for use as a pharmaceutical. The surfactant can include ionic (cationic, anionic or zwitterionic) and/or non-ionic surfactants. The use of only non-ionic surfactants is often desirable, for example due to their pH independence. The invention can thus use surfactants including, but not limited to:
the polyoxyethylene sorbitan ester surfactants (commonly referred to as the Tweens or polysorbates), such as polysorbate 20 and polysorbate 80, especially polysorbate 80;
copolymers of ethylene oxide (EO), propylene oxide (PO), and/or butylene oxide (BO), sold under the DOWFAX™, Pluronic™ (e.g. F68, F127 or L121 grades) or Synperonic™ tradenames, such as linear EO/PO block copolymers, for example poloxamer 407, poloxamer 401 and poloxamer 188;
octoxynols, which can vary in the number of repeating ethoxy (oxy-1,2-ethanediyl) groups, with octoxynol-9 (Triton X-100, or t-octylphenoxypolyethoxyethanol) being of particular interest;
(octylphenoxy) polyethoxyethanol (IGEPAL CA-630/NP-40);
phospholipids such as phosphatidylcholine (lecithin);
polyoxyethylene fatty ethers derived from lauryl, cetyl, stearyl and oleyl alcohols (known as Brij surfactants), such as polyoxyethylene 4 lauryl ether (Brij 30, Emulgen 104P), polyoxyethylene-9-lauryl ether and polyoxyethylene 12 cetyl/stearyl ether (Eumulgin™ B1, cetereth-12 or polyoxyethylene cetostearyl ether);
sorbitan esters (commonly known as the Spans), such as sorbitan trioleate (Span 85), sorbitan monooleate (Span 80) and sorbitan monolaurate (Span 20);
or tocopherol derivative surfactants, such as alpha-tocopherol-polyethylene glycol succinate (TPGS).

Many examples of pharmaceutically acceptable surfactants are known in the art e.g. see Handbook of Pharmaceutical Excipients 6th edition, 2009. Methods for selecting and optimising the choice of surfactant used in a squalene emulsion adjuvant are illustrated in Klucker, 2012. In general, the surfactant component has a HLB between 10 and 18, such as between 12 and 17, in particular 13 to 16. This can be typically achieved using a single surfactant or, in some embodiments, using a mixture of surfactants. Surfactants of particular interest include: poloxamer 401, poloxamer 188, polysorbate 80, sorbitan trioleate, sorbitan monooleate and polyoxyethylene 12 cetyl/stearyl ether either alone, in combination with each other or in combination with other surfactants. Especially of interest are polysorbate 80, sorbitan trioleate, sorbitan monooleate and polyoxyethylene 12 cetyl/stearyl ether either alone, or in combination with each other. A particular surfactant of interest is polysorbate 80. A particular combination of surfactants of interest is polysorbate 80 and sorbitan trioleate. A further combination of surfactants of interest is sorbitan monooleate and polyoxyethylene cetostearyl ether.

In certain embodiments the squalene emulsion adjuvant comprises one surfactant, such as polysorbate 80. In some embodiments the squalene emulsion adjuvant comprises two surfactants, such as polysorbate 80 and sorbitan trioleate or sorbitan monooleate and polyoxyethylene cetostearyl ether. In other embodiments the squalene emulsion adjuvant comprises three or more surfactants, such as three surfactants.

If tocopherol is present, the weight ratio of squalene to tocopherol may be 20 or less, such as 10 or less. Suitably the weight ratio of squalene to tocopherol is 0.1 or more. Typically the weight ratio of squalene to tocopherol is 0.1 to 10, especially 0.2 to 5, in particular 0.3 to 3, such as 0.4 to 2. Suitably, the weight ratio of squalene to tocopherol is 0.72 to 1.136, especially 0.8 to 1, in particular 0.85 to 0.95, such as 0.9.

If surfactant is present, typically the weight ratio of squalene to surfactant is 0.73 to 6.6, especially 1 to 5, in particular 1.2 to 4. Suitably, the weight ratio of squalene to surfactant is 1.71 to 2.8, especially 2 to 2.4, in particular 2.1 to 2.3, such as 2.2.

The amount of squalene in a single dose, such as a human dose, of squalene emulsion adjuvant is typically at least 1.2 mg. Generally, the amount of squalene in a single dose, such as a human dose, of squalene emulsion adjuvant is 50 mg or less. The amount of squalene in a single dose, such as a human dose, of squalene emulsion adjuvant may be 1.2 to 20 mg, in particular 1.2 to 15 mg. The amount of squalene in a single dose, such as a human dose, of squalene emulsion adjuvant may be 1.2 to 2 mg, 2 to 4 mg, 4 to 8 mg or 8 to 12.1 mg. For example, the amount of squalene in a single dose, such as a human dose, of squalene emulsion adjuvant may be 1.21 to 1.52 mg, 2.43 to 3.03 mg, 4.87 to 6.05 mg or 9.75 to 12.1 mg.

If tocopherol is present, the amount of tocopherol in a single dose, such as a human dose, of squalene emulsion adjuvant is typically at least 1.3 mg. Generally, the amount of tocopherol in a single dose, such as a human dose, of squalene emulsion adjuvant is 55 mg or less. The amount of tocopherol in a single dose, such as a human dose, of squalene emulsion adjuvant may be 1.3 to 22 mg, in particular 1.3 to 16.6 mg. The amount of tocopherol in a single dose, such as a human dose, of squalene emulsion adjuvant may be 1.3 to 2 mg, 2 to 4 mg, 4 to 8 mg or 8 to 13.6 mg. For example, the amount of tocopherol in a single dose, such as a human dose, of squalene emulsion adjuvant may be 1.33 to 1.69 mg, 2.66 to 3.39 mg, 5.32 to 6.77 mg or 10.65 to 13.53 mg.

If surfactant is present, the amount of surfactant in a single dose, such as a human dose, of squalene emulsion adjuvant is typically at least 0.4 mg. Generally, the amount of surfactant in a single dose, such as a human dose, of squalene emulsion adjuvant is 18 mg or less. The amount of surfactant in a single dose, such as a human dose, of squalene emulsion adjuvant may be 0.4 to 9.5 mg, in particular 0.4 to 7 mg. The amount of surfactant in a single dose, such as a human dose, of squalene emulsion adjuvant may be 0.4 to 1 mg, 1 to 2 mg, 2 to 4 mg or 4 to 7 mg. For example, the amount of surfactant in a single dose, such as a human dose, of squalene emulsion adjuvant may be 0.54 to 0.71 mg, 1.08 to 1.42 mg, 2.16 to 2.84 mg or 4.32 to 5.68 mg.

In certain embodiments the squalene emulsion adjuvant may consist essentially of squalene, surfactant and water. In certain other embodiments the squalene emulsion adjuvant may consist essentially of squalene, tocopherol, surfactant and water. Squalene emulsion adjuvants may contain additional components as desired or required depending upon the intended final presentation and vaccination strategy, such as buffers and/or tonicity modifying agents, for example modified phosphate buffered saline (disodium phosphate, potassium biphosphate, sodium chloride and potassium chloride).

High pressure homogenization (HPH or microfluidisation) may be applied to yield squalene emulsion adjuvants comprising tocopherol which demonstrate uniformly small droplet sizes and long-term stability (see EP0868918 and WO2006/100109). Briefly, oil phase composed of squalene and tocopherol may be formulated under a nitrogen atmosphere. Aqueous phase is prepared separately, typically composed of water for injection or phosphate buffered saline, and polysorbate 80. Oil and aqueous phases are combined, such as at a ratio of 1:9 (volume of oil phase to volume of aqueous phase) before homogenisation and microfluidisation, such as by a single pass through an in-line homogeniser and three passes through a microfluidiser (at around 15000 psi). The resulting emulsion may then be sterile filtered, for example through two trains of two 0.5/0.2 μm filters in series (i.e. 0.5/0.2/0.5/0.2), see WO2011/154444. Operation is desirably undertaken under an inert atmosphere, e.g. nitrogen. Positive pressure may be applied, see WO2011/154443.

International patent application WO2020160080 and Lodaya, 2019 describe squalene emulsion adjuvants comprising tocopherol which are self-emulsifying adjuvant systems (SEAS) and their manufacture.

### Vaccination Regimes, Dosing and Efficacy Criteria

Suitably, the immunogenic compositions described herein are a standard 0.5 ml injectable dose in most cases, and contain 15 μg or less, of hemagglutinin antigen component from an influenza virus strain, as measured by single radial immunodiffusion (SRD) (J. M. Wood et al.: J. Biol. Stand. 5 (1977) 237-247; J. M. Wood et al., J. Biol. Stand. 9 (1981) 317-330). Suitably the vaccine dose volume will be from 0.25 ml to 1 ml, in particular a standard 0.5 ml, or 0.7 ml vaccine dose volume. Slight adaptation of the dose volume will be made routinely depending on the HA concentration in the original bulk sample and depending also on the delivery route with smaller doses being given by the intranasal or intradermal route. Immunogenic compositions for use according to the invention may contain a low amount of HA antigen—e.g. any of 1, 2, 3, 4, 5, 6, 7, 8, 9, 10, 11, 12, 13, 14 μg of HA per influenza virus strain or which does not exceed 15 μg of HA per strain. Said low amount of HA amount may be as low as practically feasible provided that it allows formulation of a vaccine which meets the international e.g. EU or FDA criteria for efficacy. A suitable low amount of HA is from 1 to 7.5 μg of HA per influenza virus strain, suitably from 3.5 to 5 μg, such as 3.75 or 3.8 μg of HA per influenza virus strain, typically about 5 μg of HA per influenza virus strain. Another suitable amount of HA is from 0.1 to 5 μg of HA per influenza virus strain, suitably from 1.0 to 2 μg of HA per influenza virus strain, such as 1.9 μg of HA per influenza virus strain.

The influenza medicament (e.g. immunogenic composition) described herein suitably meets certain international criteria for vaccines. Standards are applied internationally to measure the efficacy of influenza vaccines.

Serological variables are assessed according to criteria of the European Agency for the Evaluation of Medicinal Products for human use (CHMP/BWP/214/96, Committee for Proprietary Medicinal Products (CPMP)) or as updated.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the recombinant HA antigen may therefore be part of a multi-dose administration regime. For example, the recombinant HA antigen may be provided as a priming dose in a multidose regime, especially a two- or three-dose regime, in particular a two-dose regime. The recombinant HA antigen may be provided as a boosting dose in a multidose regime, especially a two- or three-dose regime, such as a two-dose regime.

Priming and boosting doses may be homologous or heterologous. Consequently, the recombinant HA antigen may be provided as a priming dose and boosting dose(s) in a homologous multidose regime, especially a two- or three-dose regime, in particular a two-dose regime. Alternatively, the recombinant HA antigen may be provided as a priming dose or boosting dose in a heterologous multidose regime, especially a two- or three-dose regime, in particular a two-dose regime, and the boosting dose(s) may be different (e.g. a different HA antigen; or an alternative antigen presentation such as protein or virally vectored antigen—with or without adjuvant).

The time between doses may be two weeks to six months, such as three weeks to three months. Periodic longer-term booster doses may be also be provided, such as every 2 to 10 years.

### Methods of Treatment

In a further embodiment, the recombinant HA antigen or immunogenic composition comprising said antigen is for use in medicine, such as for use in the prevention of, or vaccination against, influenza e.g. administered to a person (e.g. subject) at risk of influenza infection.

In a yet further embodiment, the recombinant HA antigen or immunogenic composition comprising said antigen is for use in the prevention of influenza caused by a different B strain lineage than the lineage on which the HA antigen was based. For example, a B Yamagata lineage HA antigen could be used for protection against influenza caused by a non-Yamagata lineage virus e.g. a B Victoria lineage virus, or vice versa.

In a further aspect, there is provided a method of prevention and/or treatment of influenza disease, comprising the administration of a recombinant HA antigen or immunogenic composition as described herein to a person in need thereof, e.g. to a person (e.g. subject) at risk of influenza infection, e.g. an elderly person (age 50 or over, particularly age 65 or over).

In one embodiment of the above described method or use, less than 15 micrograms, such as from 3.75 to 10 micrograms of HA is administered per dose.

In one aspect, the invention provides the recombinant HA described herein at a dose of below 10 micrograms, or below 8 micrograms, or from 1-7.5 micrograms, or from 1-5 micrograms of recombinant HA for use in a vaccination regimen for the prevention of influenza, wherein the hemagglutinin sequences are from, or derived from a strain of influenza identified by an international organisation such as the WHO that monitors outbreaks of influenza virus, as associated with an outbreak or as having the potential to be associated with a future outbreak.

### Routes of Administration

The composition of the invention may be administered by any suitable delivery route, such as intradermal, mucosal (e.g. intranasal), oral, intramuscular (IM) or subcutaneous. Other delivery routes are well known in the art.

The intramuscular delivery route is particularly suitable for the immunogenic composition, in particular for the adjuvanted immunogenic composition. The composition may be presented in a mono-dose container, or alternatively, a multi-dose container. In this instance an antimicrobial preservative such a thiomersal may be present to prevent contamination during use. A thiomersal concentration of 5 μg/0.5 ml dose (i.e. 10 μg/ml) or 10 μg/0.5 ml dose (i.e. 20 μg/ml) is suitably present. A suitable IM delivery device could be used such as a needle-free liquid jet injection device, for example the Biojector 2000 (Bioject, Portland, OR). Alternatively, a pen-injector device, such as is used for at-home delivery of epinephrine, could be used to allow self-administration of vaccine. The use of such delivery devices may be particularly amenable to large scale immunization campaigns.

Intradermal delivery is another suitable route. Any suitable device may be used for intradermal delivery, for example short needle devices. Such devices are well known in the art. Intradermal vaccines may also be administered by devices which limit the effective penetration length of a needle into the skin, such as those described in WO99/34850 and EP1092444, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Also suitable, are ballistic powder/particle delivery devices which use compressed gas to accelerate vaccine in powder form through the outer layers of the skin to the dermis. Additionally, conventional syringes may be used in the classical mantoux method of intradermal administration.

Another suitable administration route is the subcutaneous route. Any suitable device may be used for subcutaneous delivery, for example classical needle. Suitably, a needle-free jet injector service is used. Such devices are well known in the art. Suitably said device is pre-filled with the liquid vaccine formulation.

Alternatively the vaccine is administered intranasally. Typically, the vaccine is administered locally to the nasopharyngeal area, suitably without being inhaled into the lungs. It is desirable to use an intranasal delivery device which delivers the vaccine formulation to the nasopharyngeal area, without or substantially without it entering the lungs.

Suitable devices for intranasal administration of the vaccines according to the invention are spray devices. Suitable commercially available nasal spray devices include Accuspray™ (Becton Dickinson). Nebulisers produce a very fine spray which can be easily inhaled into the lungs and therefore does not efficiently reach the nasal mucosa. Nebulisers are therefore not preferred.

Suitable spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO 91/13281 and EP 311 863 B and EP 516 636, incorporated herein by reference. Such devices are commercially available from Pfeiffer GmbH and are also described in Bommer, R. Pharmaceutical Technology Europe, September 1999.

Alternatively, the epidermal or transdermal vaccination route is also contemplated herein.

### EXAMPLES

### Example 1—B Strain Stem Conservation Analysis

To assess the potential for a single stem only prototype to induce cross-reactive antibodies against both Victoria and Yamagata lineage strains, 5800 non-redundant influenza B strain haemagglutinins (HA) were aligned using the MUSCLE alignment tool.

The resulting multiple sequence alignment (MSA) was used to calculate pairwise percentage identity over the 5800 sequences. On average, it was found that Yamagata and Victoria HAs share 92.2% identity. This high identity percentage already indicates potential for cross-reactivity between both lineages.

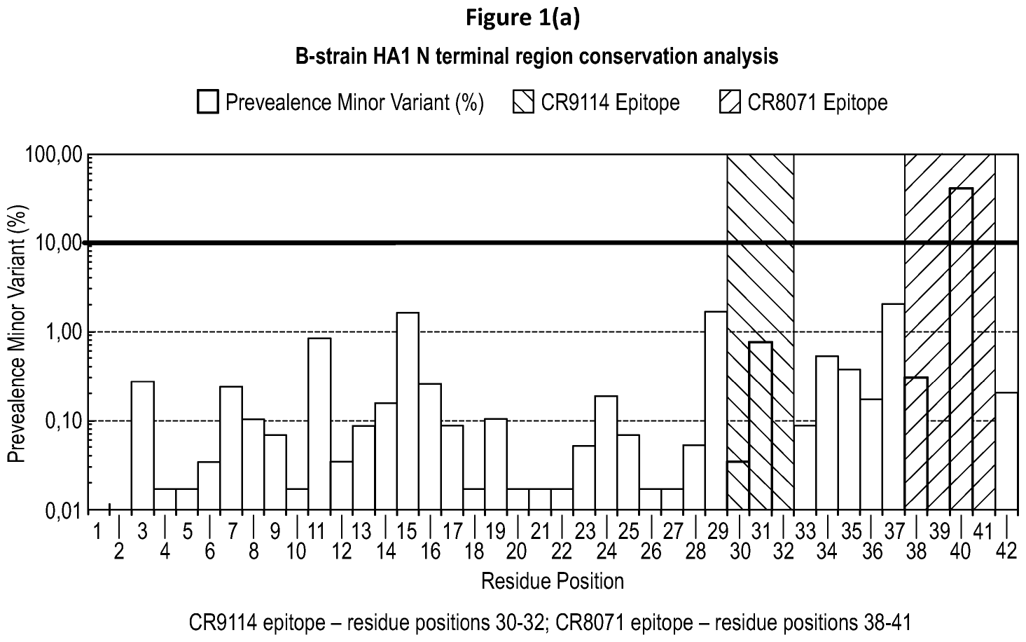

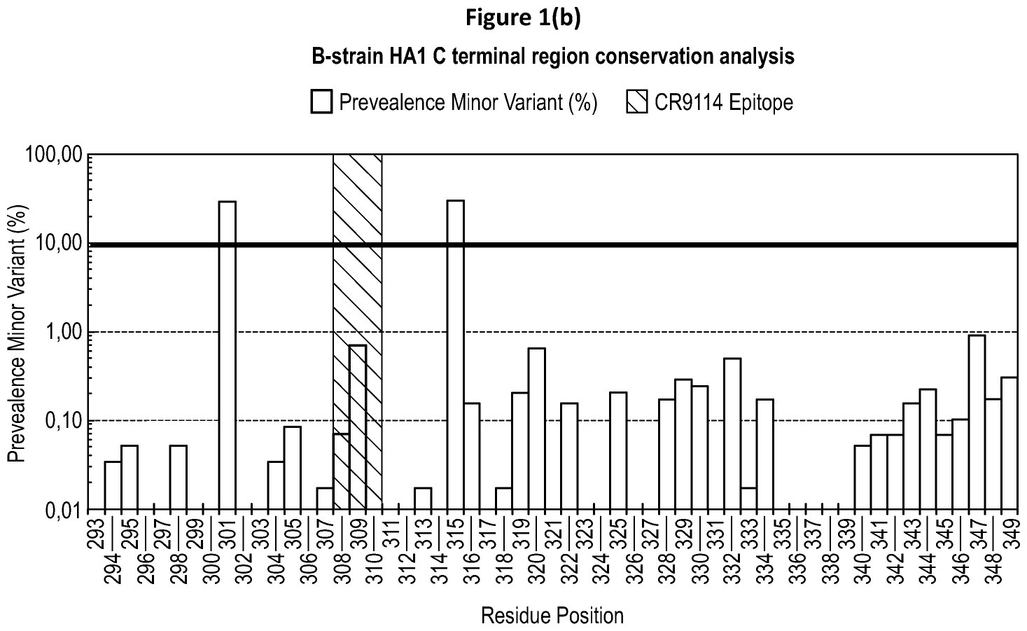

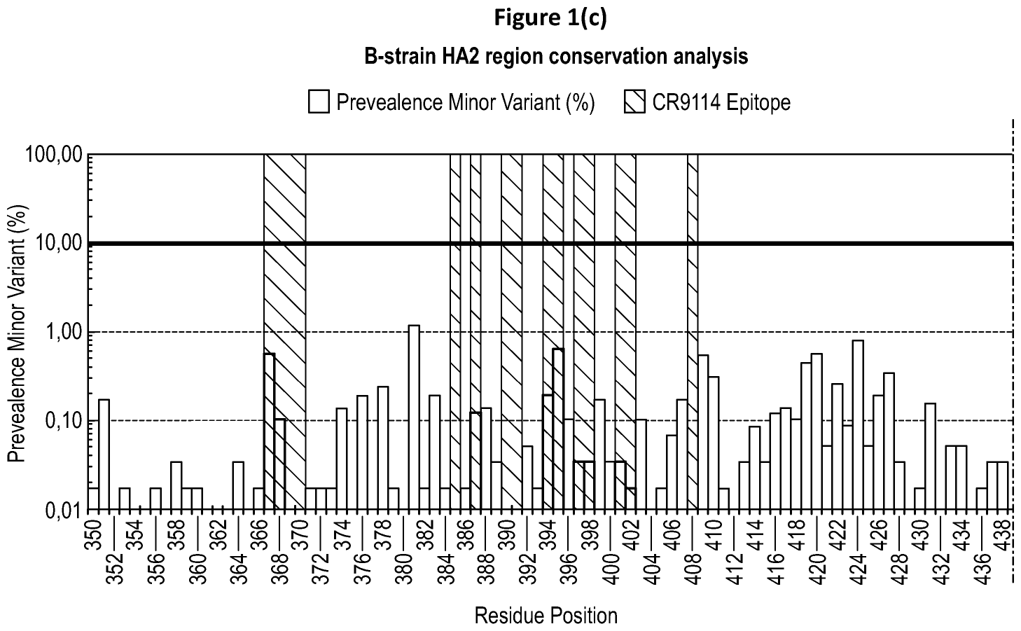

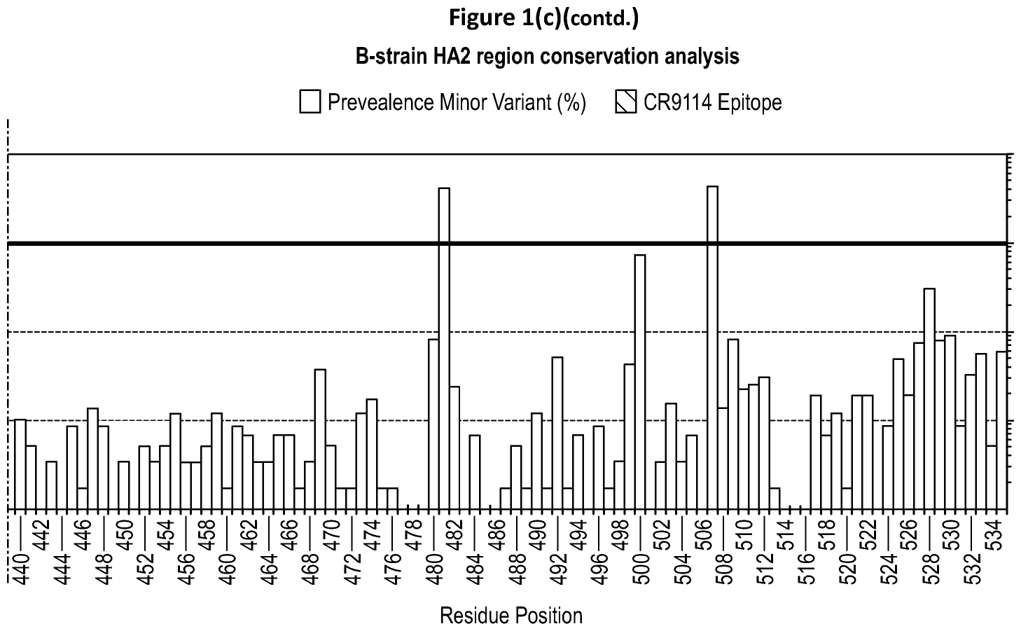

To better evaluate potential cross-reactivity for a stem construct, a deeper analysis of the HA stem regions (N-terminal HA1, C-terminal HA1 and HA2) was carried on. Overall, each region exhibits quite a high degree of conservation with over 90% of the positions found to have polymorphisms appearing with frequency below 1% over the 5800 sequences (FIG. 1). The CR9114 epitope, the so far only well-known cross-reactive stem epitope for influenza group B, was found to be also well conserved between lineages (FIG. 1).
N-terminal HA1 stem region:
   38/42 positions with polymorphism frequency <1%
   CR9114 epitope position very conserved
C-terminal HA1 stem region:
   55/57 positions with polymorphism frequency <1%
   CR9114 epitope position very conserved
HA2 stem region:
   181/186 positions with polymorphism frequency <1%
   CR9114 epitope position very conserved

Taken together, the results from the analysis confirm the potential for a single stem only construct to be able to provide cross-reactive protection against both influenza B lineages.

### Example 2—Design and Construction of the B Strain Constructs

B/Washington/02/2019 (B/Victoria lineage) strain HA 3D structure was modeled using MOE software and a validated model was used for a stem only and a full ectodomain design strategy. In both cases, for characterization purposes, the constructs were expressed as recombinant soluble proteins each containing a foldon as a trimerization domain separated from the designed HA sequence by a TEV cleavage site, and followed by a 6-His tag.
Design of a B strain stem only construct with the objective to focus immune response against HA more conserved stem epitopes.

B strain HA stem region is known to be less susceptible to mutation and exhibits a higher conservation across lineage and time compared to HA head. Among the known epitopes in HA stem, the one targeted by CR9114 (Dreyfus et al Science, 2012, 337 (6100): 1343-8) has been shown as the most cross-reactive against a panel of strains from both group A and B influenza virus.

To redirect the immune response against stem epitopes, HA engineering was performed to design stem only constructs through peptide deletion and mutation of the HA sequence in SEQ ID NO: 1 (see FIG. 2). Deletions in the HA full ectodomain included:
N58-1302 and L422-D444 (Group2)
T48-P340 and L422-D444 (Group3)
T48-P340 and V419-1451 (Group4)

Linkers selected for the peptide sequences before and after N58-1302/T48-P340 were: None, G, GS, GSG.

Linkers selected for the peptide sequences before and after L422-D444/V419-1451 were: GSG, and GSGS, GSPG, GPSG, GPSPG, GSGSG, GSGGSG [SEQ ID Nos: 53-58 respectively].

To further stabilize the trimeric conformation, N410M-S462V mutations were introduced in the prototypes. Also tested were M429P and L432P substitutions.

Some of the combinations of deletions, linkers and amino acid substitutions that were used in the stem only constructs are shown in the tables in FIG. 3. Further combinations are shown in FIG. 5(a).
Design of a stabilized B strain full ectodomain with the objective to improve manufacturability and antigenicity.

H1 haemagglutinin expressed recombinantly has been shown to be suboptimal, with difficulty maintaining its trimeric conformation. To mitigate the risk of losing the B strain ectodomain trimeric conformation and to improve both manufacturability and antigenic features, a thorough analysis of the B/Washington/02/2019 HA 3D modeled structure was performed using MOE to identify potentially interesting positions in HA2 helix B (see FIGS. 6 and 8) and its surrounding environment to introduce mutations to improve trimer conformation stability.

Using the B/Washington/02/2019 (B/Victoria lineage) strain HA 3D modeled structure, several positions were identified to mutate to attempt to stabilize the trimeric conformation:
Positions 450, 465, 472 where mutation to hydrophobic residues could promote hydrophobic exlusion and favour trimer formation, with formed trimer coiled coil structure stabilized by these hydrophobic interactions.
Positions 429 and 432, found at the end of the interloop region connecting helix A and B, for which mutation to proline could prevent post-fusion conformation formation through steric hindrance introduced by the particular side chain from proline.
Positions 410, 462 and 477 in HA2 helix A for the 1st and HA2 helix B for the last two, respectively, could be targeted to increase HA1/HA2 interaction either by hydrophobic reinforcement (410/462) or cavity filling/hydrogen bond introduction (477).

The mutations were introduced as single point mutations or in combination for a potentially additive effect. Some of the combinations of mutations that were used in ectodomain constructs are shown in the tables in FIG. 4. Further combinations are shown in FIG. 5(b).

### Example 3—Cloning, Protein Expression and Purification

### Cloning

Genes were codon optimized for human protein expression, synthesized and cloned into pmaxCloning™ vector (Lonza, Cat. VDC-1040) by GENEWIZ, using EcoRI/NotI restriction sites. The pmaxCloning™ vector backbone contains the immediate early promoter of cytomegalovirus (PCMV IE) for protein expression, a chimeric intron for enhanced gene expression and the pUC origin of replication for propagation in E. coli. The bacterial Promoter (P) provides kanamycin resistance gene expression in E. coli. The multiple cloning site (MCS) is located between the CMV promoter and the SV40 polyadenylation signal (SV40 poly A).

Each construct comprised a sequence encoding an Influenza haemagglutinin (HA) ectodomain of SEQ ID NO: 1, with mutations as shown in the table in FIG. 4, or an ectodomain of SEQ ID NO: 1 with deletions and mutations shown in FIG. 3. All constructs were fused at the C-terminus with a TEV cleavage site followed by a foldon, followed by a 6×His-tag.

### Expression

Expi293F™ cells (ThermoFisher, Cat. A14528) were used for recombinant protein expression. Cell culture and transfection were performed following manufacturer's instructions. Small scale cultures (3 mL cultures in 24-deep well plates) were used for screening of candidates and medium scale cultures (125 mL) were performed on selected top candidates.

The day before transfection, cell density and viability were assessed using a TC20™ Automated Cell Counter (Bio-Rad). Cells were seeded in fresh, prewarmed Expi293™ Expression medium (ThermoFisher, Cat. A1435102) at a density of 2·10⁶ cells/mL and cultured in a humidified 8% CO₂ incubator at 37° C. and 110 rpm. The day of the transfection, cell density and viability were assessed (viability ≥95%) and cells were diluted to a final density of 3·10⁶ cells/mL with fresh, prewarmed Expi293™ Expression medium. Transfection was performed using ExpiFectamine™ 293 Transfection Kit (Thermofisher, Cat. A14524), containing transfection enhancers and ExpiFectamine 293 transfection reagent. Briefly, plasmid DNA and transfection reagent were diluted separately in OptiMEM medium (Thermofisher, Cat. 31985062) and incubated for 5 min at RT (1 μg of plasmid DNA was used per 1 mL of cell culture). Both mixtures were then combined and incubated for 20 additional min at RT. The ExpiFectamine™ 293/plasmid DNA complexes solution was then carefully added to the cells. Cells were cultured in a humidified 8% CO₂ incubator at 37° C. and 110 rpm. On day 1 post-transfection (18-22h post-transfection), ExpiFectamine™ 293 Transfection Enhancers 1 and 2 were added. On day 4 post-transfection cells were harvested by centrifugation at 4° C. and 5000×g for 10 min. Cell pellets were discarded and supernatants were supplemented with Complete™ Protease Inhibitor Cocktail (Roche, Cat. 11697498001). Protein expression was checked by SDS-PAGE and Western blot before purification (data not shown).

### Purification

Purification on HTP expression (2.5 mL culture on a 24 Deep Well format) was performed by adding 200 μL of Nickel Sepharose Excel (GE) slurry preequilibrated in buffer A (20 mM Bicine, 500 mM NaCl, 20 mM Imidazole, pH 8.3) with 0.2 mM 4-(2-aminoethyl)benzenesulfonyl fluoride hydrochloride (AEBSF) (Sigma) and 20 mM Bicine pH 8.3. After rocking at 900 rpm overnight, the samples were transferred to a 96 DW Thompson filter plate and washed 3 times with 1 mL of buffer A under negative pressure. The proteins were eluted by centrifugation (10 minutes at 800 g) with 2 times 110 μL of buffer B (20 mM Bicine, 500 mM NaCl 500 mM Imidazole, pH 8.3), desalted by PD multitrap G-25 and analysed by SDS-PAGE.

Purification on medium scale expression (125 mL culture) was performed by gravity flow column packed with 3 mL of Nickel Sepharose Excel (GE) preequilibrated in buffer A (20 mM Bicine, 500 mM NaCl, 20 mM Imidazole, pH 8.3). After sample loading, the resins were washed with 15 CV of buffer A and the proteins were eluted with 4CV of buffer B (20 mM Bicine, 500 mM NaCl 500 mM Imidazole, pH 8.3). The proteins were then concentrated using Vivaspin 20 with a cut-off of 10 KDa at 3000 g at 4° C. The concentrated samples were loaded onto Superdex 200 increase 10/300 (GE) or Superdex 200 16/600 (GE) equilibrated in buffer C (20 mM bicine, 150 mM NaCl, pH 8.3) with a flow rate of 0.75 ml/min. Fractions corresponding to the proteins of interested were pooled together, filtered 0.22 μM and stored at −80° C.

Protein concentrations were determined by RCDC assay (Biorad) and the purity by SDS-PAGE. A sample set of the medium scale protein purification analysis carried out by SDS-PAGE is shown in FIG. 10: a) proteins corresponding to different truncated HA ectodomain mutants and b) different HA ectodomain mutants. MW, molecular weight marker; BMP, purified protein ID; μg, amount of purified protein loaded (in μg).

### Characterization

### UHPLC—Ultra High Performance Liquid Chromatography

The stability of the trimer assembly of the semi-purified HA constructs from the high throughput screening (HTS) experiments was assessed by HPLC-SEC-UV. Briefly, 10 μl of each preparation was injected on a Superdex 200 increase 5/150 GL column (Cytiva ref. 2899094) at 0.5 ml/min. UV at 280 nm was recorded during the 10 minutes run. The column was maintained at 30° C. and samples at 8° C. during the experiment. Elution times of HA peaks were compared to calibration standards (Waters BEH200 SEC Protein Standard Mix, ref. Waters 186006518). Based on retention times, peak areas in predefined regions of elution for aggregates, oligomers, trimers and monomers respectively, were recorded.

### BLI—Bio-Layer Interferometry

An Octet Red instrument (Pall-ForteBio, Menlo Park, USA) was used for all the IgG binding measurements. All measurements were made in Kinetics Buffer (KB) (Pall-ForteBio, Menlo Park, USA) 1× and a constant agitation of 1000 rpm was kept constant. Mutant proteins were prepared by diluting 50 μl of the protein solution in 150 μl of KB 1× and immobilised on Ni-NTA sensortips for 180 seconds. Washing of unbound ligand was performed by incubation of sensortips in buffer solution for 60 seconds. Binding to fAb CR9114 was monitored upon immersion into a 20 μg/ml solution of CR9114 in KB for 120 seconds. Dissociation was monitored for 180 seconds upon immersion in KB 1× buffer.

### NanoDSF—Nano Differential Scanning Florimetry

Protein solutions were loaded in glass capillaries and submitted to a linear heating process (20 to 95° C. at 1° C./min) inside a NanoDSF NT-Plex instrument (Nanotemper Technologies, Munich, Germany). The fluorescence intensity at 330 nm and 350 nm was constantly recorded during the heating process. First derivative of the fluorescence ratio (350 nm/330 nm) plotted against the temperature was used to determine the temperature of melting Tm.

### AUC

Sedimentation velocity analytical ultracentrifugation (SV-AUC) was performed to determine the molecular weight and stoichiometry of the proteins by measuring the rate at which molecules move through the buffer in response to centrifugal force.

SV-AUC was performed using a Beckman-Coulter Optima AUC analytical ultracentrifuge with an AN-60Ti rotor and the protein was at 0.5 mg/ml and frozen at −80° C. before experiment. The selected rotor speed for the run was 20,000 rpm, the temperature was maintained at 20° C. and the absorbance profile at 280 nm was recorded every min.

Protein-specific density and solvent density were calculated by using the software SEDNTERP 1 (Sedimentation Interpretation Program Version 1.11). The dataset was analysed with Sedfit 15.01b program using a continuous size distribution c(s) model.

### Example 4—Batch 1 Results

A first batch of constructs was designed, prepared and assessed according to the previous Examples. In all, 80 stem only constructs and 127 ectodomain constructs were investigated. For lead constructs, read-outs from characterisation studies described in Example 3 are shown here. The read-outs were combined to produce an aggregated view of the data in Table 2. Unless otherwise noted, experiments were carried out on stem and ectodomains with trimerisation domain present.

TABLE 2
Consolidated view of characterisation read-outs for the midscale production of preselected influenza
B strain constructs. The selected candidates are marked by crosses in the last column.

| | | | | | | | | OCTET | | |
|---|---|---|---|---|---|---|---|---|---|---|
| | | | | | | | | CR9114 | | |
| | | | | AUC | | quantity | productivity | 30° C. | NanoDSF | |
| | FluB | BMP | UPLC | no TEV S | TEV S | purified | total qty | response | Tm1 | Selected |
| | # | # | oligomer | (S) | (S) | mg | mg/L | (nm) | ° C. | Mutants |
| STEM | 18 | 1383 | Aggreg | aggr | — | 2.2 | 17.9 | 0.00 | 79.8 | |
| | 29 | 1384 | Aggreg | aggr | — | 3.6 | 28.8 | 0.00 | 80.9 | |
| | 35 | 1385 | Aggreg | 3m | 1m + aggr | 1.0 | 8 | 0.00 | 80.4 | |
| | 36 | 1386 | Aggreg | 3m | 1m + aggr | 0.9 | 7 | 0.01 | 79.9 | |
| | 75 | 1387A | Aggreg | ND | — | 1.0 | 7.9 | 0.00 | 81.0 | x |
| | 75 | 1387B | 1m/3m | 3m | 1m + 3m | 0.6 | 4.6 | −0.02 | 79.3 | x |
| | 77 | 1388A | Aggreg | ND | — | 1.9 | 15 | 0.01 | 81.0 | x |
| | 77 | 1388B | 1m/3m | 3m | 1m + 3m | 0.6 | 4.7 | 0.01 | 79.3 | x |
| | 80 | 1389 | 3m | 9 | — | 0.9 | 7.3 | 0.00 | 80.8 | |
| ECTO | 83 | 1390 | 3m | 3m | 1m | 16.0 | 128 | 0.59 | 57.3 | |
| | 115 | 1391 | 3m | 3m | 3m | 12.0 | 96 | 0.17 | 65.5 | x |
| | 159 | 1392 | 3m | 3m | 3m | 10.0 | 80 | 0.69 | 61.0 | x |
| | 81 | 1394 | 1m | 3m | 1m | — | — | 0.57 | 53.8 | |
| | 82 | 1395 | 1m | 3m | 1m | 22.0 | 176 | 0.48 | 53.6 | x |
| | 84 | 1396 | 3m | 3m | 3m | 6.0 | 48 | 0.35 | 63.2 | |
| | 87 | 1397 | 3m | 3m | 3m | 4.1 | 32.6 | 0.43 | 62.2 | |
| | 90 | 1398 | 3m | 3m | 3m | 5.5 | 44 | 0.50 | 60.9 | |
| | 111 | 1399 | 3m | 3m | 3m | 12.5 | 100 | 0.13 | 64.2 | |
| | 112 | 1400 | 3m | 3m | 3m | 11.0 | 88 | 0.19 | 65.7 | |
| | 119 | 1401 | 3m | 3m | 3m | 2.5 | 20 | 0.16 | 66.3 | |
| | 136 | 1402 | 3m | 3m | 3m | 15.0 | 120 | 0.17 | 66.0 | x |
| | 161 | 1403 | 3m | 3m | 3m | 7.0 | 56 | 0.20 | 65.6 | |
| | 182 | 1404 | 3m | 3m | 3m | 6.0 | 48 | 0.20 | 65.0 | |
| | 187 | 1405 | 3m | 3m | 3m | 6.0 | 48 | 0.11 | 62.1 | |
| | 188 | 1406 | 3m | 3m | 3m | 8.0 | 64 | 0.08 | 65.2 | |
| | 203 | 1407 | 3m | 3m | 3m | 5.0 | 40 | 0.09 | 63.7 | |
| CTRL | Wash | 1378 | 3m | 3m | 3m | 90 | 90 | 0.48 | 62.4 | |

### Key for Table 2:

3m: trimer; 1m: monomer; ND: not detected; Oligo: aggregated of smaller order; Aggreg: high order aggregate;
The control shown at the bottom of Table 2 BMP1378 is purified B/Washington/02/2019 HA, with the same backbone as for the ectodomain constructs: full ectodomain fused with TEV cleavage motif, trimerisation domain and His tag.
“No TEV” means before cleavage, “TEV” means after cleavage (TEV protease was used to cleave the trimerization domain from the HA part of the construct).

### High-Throughput Screening:

The 207 batch 1 mutant candidates (FIGS. 5(a) and (b)) expressed at small scale and partly purified were characterised with the following read-outs:
Productivity: The quantity of protein (expressed in mg/L) after purification was measured by colorimetric method. Previous analysis suggested that higher productivity is often associated with a more stable folding. We used this parameter as a selection criterion for full ectodomain constructs. Stem constructs did not express similarly to full ectodomain and were not selected against this parameter.
UHPLC-SEC: to discriminate between soluble forms of the proteins (either monomer, trimer, higher order oligomers or soluble aggregates). Each form displays as a (partly) separated peak on the elution profile.
BLI: biosensor technology used to quantify the binding of structure-specific immuno tools to the immobilised mutants. Here, fAb of the CR9114 monoclonal antibody was used to probe the stem region of the proteins in comparison to the control B/Washington/02/2019. The binding was recorded as an increase of the thickness (expressed in nm) of the sensing surface as the antibody ligand binds to the immobilised HA protein mutant. In previous experiments, we observed an increase of the binding to more stable HA trimers.
NanoDSF: protein folding fingerprinting technology used to confirm the tightness of protein folding by measuring the defolding temperature (Tm, expressed in ° C.) compared to that of the reference B/Washington/02/2019. This read-out is used to ensure that the mutation pattern does not alter the protein folding.

Overall, stem constructs displayed poor productivity, a prominent aggregation pattern upon UHPLC, and relatively poor binding to fAb CR9114. Selection of leads in this group of constructs was therefore predominantly based on their expression level, their relative BLI binding response, as well as theoretical basis (intended impact of specific mutation). 7 constructs were selected.

Full ectodomain constructs showed a more favourable UHPLC separation pattern and were selected based on the observation of stable trimers, as well as binding response to CR9114 in BLI experiments. Combining these analytical attributes as well as a review of the theoretical impact of the mutation was used to select lead candidates.

Sample plots for protein characterization by NanoDSF (before foldon removal by TEV cleavage) are shown in FIG. 12: a) thermal unfolding fingerprinting obtained for full ectodomain construct BMP1399, b) thermal unfolding profile obtained for full ectodomain construct BMP1395 and c) thermal unfolding profile obtained for stem construct BMP1387B.

In all cases, nanoDSF was not considered as a selection tool, but rather for the confirmation of the preservation of the protein folding similar to the reference.

After selection of 24 candidates (7 stem and 17 full ectodomain constructs), midscale expression and purification were performed both to confirm the productivity of each selected candidate and to provide more protein material for characterization purposes. At midscale, the following characterization assays were performed:
AUC replaced UHPLC for assessment of the trimer conformation. This read-out offers higher particle size separation power, but at the expense of sample consumption, and is therefore not compatible with HTS. Using AUC, foldon-containing (TEVs) and foldon-cleaved (no TEVs) samples were compared to infer first the occurrence of a trimer conformation, as well as its stability in the absence of the foldon trimerisation domain.
BLI was repeated on foldon-containing samples using fAb CR9114 as a probe for the conformation of the stem region of the constructs, relative to the reference.

Sample results for BLI are shown in FIG. 11: a) and b) are ectodomain and c) and d) stem only. Consecutive steps of the BLI workflow were as follows: 1. Baseline signal (sensors in buffer); 2. loading of full ecto or stem candidate onto the sensing surface (sensors in protein solution); 3. second baseline (sensors in buffer) suggesting stable loading of the protein analyte; 4. association phase showing the interaction of protein analyte with CR9114 ligand (sensors in CR9114 solution); 5. dissociation phase (sensors in buffer) showing unbinding of CR9114 from the analyte.

Sample results for medium scale protein characterization by SV-AUC are shown in FIG. 13: a) sedimentation profile obtained for full ectodomain construct BMP1399 before and after foldon removal by TEV cleavage; b) sedimentation profile obtained for full ectodomain construct BMP1395 before and after foldon removal by TEV cleavage; and c) sedimentation profile obtained for stem construct BMP1387B before and after foldon removal by TEV cleavage.

Analysis of the stem candidates again showed poor productivity, and prominent aggregation. Two constructs showed a partial trimer conformation in solution, and a partial monomerisation upon foldon removal. Because all the other constructs were aggregated, these 2 constructs were selected.

Analysis of the full ectodomain candidates expressed at midscale showed the expected profile of trimers that were stable upon foldon removal, except for 2 constructs. Most of these constructs exhibited either productivity equivalent or superior to the reference batch.

### Example 5—Batch 2 Results

A second batch of constructs was designed, prepared and assessed according to the previous Examples, with some additional deletions and mutations incorporated.
B strain stem only construct

HA engineering was performed to design further stem only constructs through peptide deletion and mutation of the HA sequence in SEQ ID NO: 1 (see FIG. 2). Deletions from the HA full ectodomain included:
N58-1302 and L422-D444 (Group2)
T48-P340 and L422-D444 (Group3)
T48-P340 and V419-1451 (Group4)
T48-P340 and S413-A448 (Group 3 variation)
T48-P340 and S413-1451 (Group 4 variation)

Linkers selected for the peptide sequences before and after N58-1302/T48-P340 were: GS, GSG.

Linkers selected for the peptide sequences before and after L422-D444/V419-1451/S413-A448/S413-1451 were: GSGS [SEQ ID NO: 53], GSPG [SEQ ID NO: 54], GSGSG [SEQ ID NO: 57], GSGGSG [SEQ ID NO: 58], DVANKVSKATDGSG [SEQ ID NO: 59], DVANKVSKATDGSGG [SEQ ID NO: 60], DVANKVSKAGS [SEQ ID NO: 61] and DVANKVSKAGG [SEQ ID NO: 62].

To further stabilize the trimeric conformation, mutations were introduced in the prototypes as for batch 1, plus additional mutations selected from: V419S, L446S/Q/D, R447L, D449K, T450V, 1451N, G465V/L/F and L477Q.

Some of the combinations of deletions, linkers and amino acid substitutions that were used in the stem only constructs are shown in the table in FIG. 3(b) and FIG. 5(d).
B strain full ectodomain construct

Further ectodomain constructs were designed using combinations of mutations selected from the same list of positions and mutations as for Example 2.

Some of the combinations of mutations that were used in ectodomain constructs of batch 2 are shown in the table in FIG. 4 and in FIG. 5(c).

In all, 148 stem only constructs and 50 ectodomain constructs were investigated in this batch. High throughput screening was carried out as described in Example 4. For lead constructs from batch 2, read-outs from characterisation studies described in Example 3 are shown here. The read-outs were combined to produce an aggregated view of the data in Table 3 below. Unless otherwise noted (e.g. AUC TEV experiment), experiments were carried out on stem and ectodomains with trimerisation domain present. The 198 constructs from batch 2 are shown in FIG. 5(c) Ectodomain and (d) Stem only.

TABLE 3
Consolidated view of characterisation read-outs for midscale production of preselected influenza B
strain constructs from Batch 2. The selected candidates are marked by crosses in the last column.

| | | | | | | | OCTET | | | | |
|---|---|---|---|---|---|---|---|---|---|---|---|
| | | | | | Quantity | | Fab | | | | |
| | | | AUC | | quantity | Productivity | CR9114 | NanoDSF | | | |
| | | | Conformation | | purified | Yield (mg/L) | response | Tm1 | Tm2 | Tm3 | Selected |
| | FluB | BMP | no TEV cleaved | TEV cleaved | mg | mg/L | nm | ° C. | ° C. | ° C. | Mutants |
| STEM | Flu289 | 1422 | Trimer | Monomer | 1.5 | 12.4 | −0.02 | 79.0 | | | |
| | Flu295 | 1423 | Trimer | Monomer | 1.0 | 7.7 | −0.03 | 79.0 | | | |
| | Flu303 | 1424 | Trimer | Monomer | 6.4 | 51.3 | −0.02 | 82.0 | | | |
| | Flu309 | 1425 | Trimer | Monomer + Dimer | 6.9 | 55.1 | −0.02 | 83.2 | | | |
| | Flu330 | 1426 | Trimer | Monomer + Dimer | 2.5 | 20.3 | −0.02 | 82.0 | | | |
| | Flu333 | 1446 PII | Trimer | Monomer + Dimer | 1.3 | 10.4 | 0.00 | 83.1 | | | |
| | Flu337 | 1427 | Trimer | Monomer + Dimer | 2.5 | 19.7 | −0.03 | 84.1 | | | |
| | Flu378 | 1428 | Trimer | Monomer | 0.9 | 7.5 | −0.03 | 80.2 | | | |
| | Flu389 | 1429 | Trimer | Monomer | 1.6 | 12.4 | −0.02 | 77.0 | | | |
| | Flu392 | 1430 | Trimer | n/a | 1.5 | 11.7 | −0.04 | 78.5 | | | |
| | Flu408 | 1431 | Trimer | Monomer | 1.0 | 7.7 | −0.05 | 79.3 | | | |
| | Flu412 | 1432 | Trimer | Monomer | 0.9 | 7.3 | −0.04 | 79.1 | | | |
| ECTO | Flu209 | 1434 | Trimer | Trimer | 16.7 | 133.2 | 0.25 | 65.1 | | | x |
| | Flu212 | 1435 | Trimer | Trimer | 14.8 | 118.0 | 0.29 | 63.5 | | | x |
| | Flu227 | 1436 | Trimer | Monomer | 17.8 | 142.4 | 0.27 | 49.8 | 62.2 | 76.4 | |
| | Flu245 | 1437 | Trimer | Trimer + Monomer | 14.9 | 119.2 | 0.17 | 64.4 | | | |
| | Flu250 | 1438 | Trimer | Trimer | 12.4 | 98.8 | 0.23 | 64.6 | | | |
| | Flu251 | 1439 | Trimer | Trimer | 5.9 | 46.8 | 0.20 | 64.9 | | | |
| | Flu254 | 1440 | Trimer | Trimer + Monomer | 14.15 | 113.2 | 0.24 | 65.1 | | | x |
| | Flu256 | 1441 | Trimer | Trimer | 13.3 | 106.0 | 0.24 | 65.5 | | | x |
| | Flu260 | 1442 | Trimer | Trimer + Monomer | 11.2 | 89.6 | 0.22 | 64.5 | | | |
| | Flu262 | 1443 | Trimer | Trimer | 12.8 | 102.4 | 0.21 | 65.6 | | | x |
| | Flu264 | 1444 | Trimer | Monomer | 12.9 | 103.2 | 0.13 | 50.3 | 62.2 | 77.2 | |
| | Flu266 | 1445 | Trimer | Monomer | 4.9 | 39.2 | 0.10 | 51.0 | 63.4 | 78.2 | |
| Ctrl | Wash19 | 1378 | Trimer | Trimer | 90.0 | 90.0 | 0.38 | 62.4 | — | — | |
| WT | | | | | | | | | | | |

### Key for Table 3:

The control shown at the bottom of Table 3 BMP1378 is purified B/Washington/02/2019 HA, with the same backbone as for the ectodomain constructs: full ectodomain fused with TEV cleavage motif, trimerisation domain and His tag.
“No TEV cleaved” means before cleavage, “TEV cleaved” means after cleavage (TEV protease was used to cleave the trimerization domain from the HA part of the construct).

### Example 6—Batch 3 Results

A third batch of constructs was designed, prepared and assessed according to the previous Examples, with some additional deletions and amino acid substitutions incorporated. This batch was made up of stem only constructs, using two constructs from Batch 2: Flu337 and Flu309, as the basis or ‘backbone’ for further prototypes, all containing the amino acid substitutions: N410M-S462V-G465F-L477Q. Additional substitutions were present in the Flu309 backbone constructs: L446S-R447A. Selected constructs are shown in FIG. 3(c).

Further linkers were employed in these constructs. Linker 1 was selected from: ASTG, IPGTGT, LPTS, YADGG, LKGTGG, QSGG, TPSGS, DOTTQT, IAGPQGSV, LTVNGEDVG, VAIPNTSYVV, FENNLFRA, VAFNNDNNQ, STYAAIGNAL [SEQ ID NOS: 63-76 respectively]. Linker 2 was selected from: SKILTGDYTS, SAFYEAFPGAT, SATLDEIPSLKE, SESFKQTEGQPA, SETVAKWEKQLAAGDP, SNAMSKLAQQFTDQPTP [SEQ ID NOS: 77-82 respectively].

### Example 7—Mouse Immunogenicity Studies

### Suggested Study

The immunogenicity of influenza B strain stem and ectodomain vaccine candidates is evaluated in CB6F1 mice. Female CB6F1 mice are immunized at days 0 and 28 with:
(a) recombinant stem or ectodomain (e.g. SEQ ID Nos: 2-25 or SEQ ID Nos: 84-121 or SEQ ID Nos: 160-167) without adjuvant;
(b) recombinant stem or ectodomain (e.g. SEQ ID Nos: 2-25 or SEQ ID Nos: 84-121 or SEQ ID Nos: 160-167) adjuvanted with for example 25 μL AS03;
(c) QIV or TIV (commercially available quadrivalent or trivalent influenza vaccine comprising inactivated split influenza virions) without adjuvant;
(d) QIV or TIV formulated with adjuvant as for (b); or
(e) NaCl solution.

Serum samples are collected and analysed using suitable assay protocols, such as those described below.

### Study A

The immunogenicity of “HA Rix” B constructs (full ectodomain and stem-only HA proteins, all containing foldon and based on B/Washington/2/2019 sequence) was evaluated in BALB/c mice. Eight female BALB/c mice were immunized at Day 0 and Day 28 with:
(a) Full ectodomain HA (wild-type sequence), 0.2 μg/dose, adjuvanted with AS03A, −1/10 human dose (HD)
(b) Full ectodomain HA (Flu115), 0.2 μg/dose, adjuvanted with AS03A, −1/10 human dose (HD)
(c) Full ectodomain HA (Flu159), 0.2 μg/dose, adjuvanted with AS03A, −1/10 human dose (HD)
(d) Full ectodomain HA (Flu209), 0.2 μg/dose, adjuvanted with AS03A, −1/10 human dose (HD)
(e) Full ectodomain HA (Flu256), 0.2 μg/dose, adjuvanted with AS03A, −1/10 human dose (HD)
(f) Stem-only HA (Flu460), 0.2 μg/dose, adjuvanted with AS03A, −1/10 human dose (HD)
(g) Stem-only HA (Flu460), 2 μg/dose, adjuvanted with AS03A, −1/10 human dose (HD)
(h) Stem-only HA (Flu474), 0.2 μg/dose, adjuvanted with AS03A, −1/10 human dose (HD) (this antigen was only tested at a dose of 0.2 μg, as there was not enough material to test at the higher dose)
(i) Stem-only HA (Flu490), 0.2 μg/dose, adjuvanted with AS03A, −1/10 human dose (HD)
(j) Stem-only HA (Flu490), 2 μg/dose, adjuvanted with AS03A, −1/10 human dose (HD)
(k) Stem-only HA (Flu523), 0.2 μg/dose, adjuvanted with AS03A, −1/10 human dose (HD)
(l) Stem-only HA (Flu523), 2 μg/dose, adjuvanted with AS03A, −1/10 human dose (HD)
(m) QIV 2021/2022 (commercially available quadrivalent influenza vaccine from GSK comprising inactivated split influenza virions of the strains A/Victoria/2570/2019 (H1N1), A/Tasmania/503/2020 (H3N2), B/Washington/02/2019 (B/Victoria) and B/Phuket/3073/2013 (B/Yamagata), 0.2 μg/strain/dose, adjuvanted with AS03A, −1/10 human dose (HD)

Note, the sample size of the QIV adjuvanted group was eventually reduced to 7, as one mouse died during the course of the study. The remaining volume for one serum sample of group 2 (Flu115) was not sufficient to perform ELISA, so there were only 7 values available in this group for these assays.

Serum samples were collected at day 42 (corresponding to 14 days post second immunization) and analyzed as described in Examples 9 to 12 using the assay protocols described in Example 8.

### Study B

Another study, similar to Study A, was conducted to compare stem-only HA constructs with or without ferritin-based nanoparticularization. The proteins expressed on ferritin nanoparticles did not contain foldon. Eight female BALB/c mice were immunized at Day 0 and Day 28 with:
(a) Stem-only HA on ferritin nanoparticles (Flu609), 2 μg/dose, adjuvanted with AS03A, −1/10 human dose (HD)
(b) Stem-only HA on ferritin nanoparticles (Flu609), 0.2 μg/dose, adjuvanted with AS03A, −1/10 human dose (HD)
(c) Stem-only HA with foldon (Flu490, equivalent HA sequence to Flu609), 2 μg/dose, adjuvanted with AS03A, −1/10 human dose (HD)
(d) Stem-only HA on ferritin nanoparticles (Flu614), 2 μg/dose, adjuvanted with AS03A, −1/10 human dose (HD)
(e) Stem-only HA on ferritin nanoparticles (Flu614), 0.2 μg/dose, adjuvanted with AS03A, −1/10 human dose (HD)
(f) Stem-only HA with foldon (Flu523, equivalent HA sequence to Flu614), 2 μg/dose, adjuvanted with AS03A, −1/10 human dose (HD)
(g) QIV 2021/2022 (commercially available quadrivalent influenza vaccine from GSK comprising inactivated split influenza virions of the strains A/Victoria/2570/2019 (H1N1), A/Tasmania/503/2020 (H3N2), B/Washington/02/2019 (B/Victoria) and B/Phuket/3073/2013 (B/Yamagata), as a commercial comparator, 1.5 μg/strain/dose (corresponding to 1/10 human dose), not adjuvanted
(h) QIV 2021/2022, 0.2 μg/strain/dose), adjuvanted with AS03A, −1/10 human dose (HD)

Serum samples were collected at day 42 (corresponding to 14 days post second immunization) and analyzed as described in Example 9 using the assay protocols described in Example 8.

### Example 8—Assay Protocols

### Anti-Hemagglutinin (HA) IgG Serology ELISA

Quantification of mouse anti-HA IgG antibodies was performed by ELISA using recombinant HA (full ectodomain or stem-only proteins) as coating antigens diluted to reach a concentration of 8 μg/ml (for full ectodomain proteins) or 5 μg/ml (for stem-only protein) in PBS (50 μl/well), and adsorbed overnight at 4° C. in 96-well microtiter plates (Maxisorb Immunoplate Nunc 439454). The plates were then incubated for 1 hour at 37° C. with 100 μl/well of PBS+10% milk (saturation buffer). Twelve two-fold dilutions of sera (diluted in PBS+1% BSA+0.1% Tween 20) were added to the coated plates (50 μl/well), and incubated for 90 minutes at 37° C. The plates were then washed four times with PBS+0.1% Tween 20. Peroxydase-conjugated goat anti-mouse IgG (Jackson 115-035-003) diluted 1/200 in dilution buffer was added to each well (50 μl/well), and incubated for 1 hour at 37° C. After another washing step, plates were incubated 20 minutes at RT with OPDA substrate (Sigma P4664). The reaction was stopped with H2SO4 2N, and optical densities were read at 490-620 nm. The titers were expressed as ELISA 50% endpoint titers corresponding to the dilution of sample corresponding to an optical density of 1.5 (50% of the high plateau). In the absence of detection of binding activity, the corresponding sample was assigned an arbitrary titer corresponding to half the first serum dilution (1:100), namely 50.

### Hemagglutination Inhibition Assay (HI)

The principle of the HA1 assay is based on the ability of specific anti-influenza antibodies to inhibit hemagglutination of red blood cells (RBC) by influenza virus hemagglutinin (HA). Sera were first treated, to remove non-specific inhibitors, with Receptor Destroying Enzyme (Sigma cat. C-8772) at a concentration of 2% (incubation 18H at 37° C.), heat-inactivated 30 min at 56° C. and treated with chicken RBC at a concentration of 5% (incubation 1h at +4° C.). After pre-treatment, two-fold dilutions of decanted sera were incubated 30 min at RT with 4 hemagglutination units of whole influenza virus (egg-derived whole inactivated influenza). Chicken RBC were then added at a concentration of 0.5% and the inhibition of hemagglutination was scored. The titers were expressed as the reciprocal of the highest dilution of serum that fully inhibited hemagglutination. As the first dilution of sera was 1:20, a titer of 10 was used for samples below the limit of detection.

### In Vitro Influenza Neutralization Assay

Neutralization Assay was performed using the following infectivity medium: Ultra-MDCK medium (BioWhittaker cat. BE12-749Q) supplemented with 1% Penicillin-Streptomycin (Invitrogen cat. 15140-122) and 2 μg/ml TPCK-treated trypsin (Sigma cat. T1426). Twelve two-fold serial dilutions of sera were prepared in monoplicate in flat bottom 96-well culture plates (Nunc cat. 167008). Sera were mixed with an equal volume (50 μL/well) of influenza viruses (egg-derived influenza virus) diluted in infectivity medium to reach 100 TCID50/well (with an acceptance range of 30-300), and were incubated at room temperature (RT) for 1 h 30. One column was used as the virus only control, and one column as the cells only control. After incubation, MDCK cells suspension was prepared and added at a concentration of 24000 cells to each well. Plates were incubated at 35° C., 5% CO₂ for 5 days. After incubation, the presence of the virus was detected using an hemagglutination test. 50 μl of supernatant were transferred in V-bottom 96 wells plates (Greiner cat. 1651101), and 50 μl of a 0.5% chicken red-blood cells suspension were added to each well. After an incubation of 1h at RT, the hemagglutination was observed and the neutralization titers were determined, corresponding to the highest serum dilution for which no hemagglutination was observed. As the first dilution of sera was 1:20, a titer of 10 was used for samples below the limit of detection.

### Antibody Dependent Cell Cytotoxicity (ADCC) Reporter Bioassay (Promega)

For determination of ADCC functionality, the mouse FcgRIII kit from Promega was used, with the following protocol. Serial dilutions of sera were prepared in 96-well plates. Target cells (Expi293 cells transfected in house to express hemagglutinin stem antigen from B/Washington/2/2019 on their surfaces) were added to each well (24000 cells/well). Effector cells (Jurkat cells, from the kit, transfected with an enzymatic pathway inducing bioluminescence when activated by antigen-antibody-FcgRIII complex) were also added to each well (60000 cells/well), and incubated 6 hours at 37° C. Luciferase activity was then measured, after having applied the Bio-Glow substrate (provided in the kit), using a Luminescence plate reader. Results were expressed as Area Under the Curve (AUC).

### Example 9—Anti-HA IgG Antibodies by ELISA at 14 Days Post Dose 2

Serum titers in IgG antibodies binding to 3 different full ectodomain HA (2 from B/Victoria group and 1 from B/Yamagata group) and, for study A, to one stem only HA (expressed on nanoparticles to avoid the detection of anti-foldon antibodies) were measured by ELISA at 14 days post second immunization (day 42). The results from Study A are shown in FIGS. 14 to 17, and the results from Study B are shown in FIGS. 18 to 20.

In each figure, individual titers values are shown together with the geometric mean titers (GMT) and 95% confidence interval (95Cl).

### Conclusions for Study a:

When a full ectodomain HA from B/Victoria group was used for the coating (FIGS. 14 and 15), a better, although highly variable, antibody response was measured in the sera of the groups that had been immunized with a full HA antigen in comparison to the groups that had been immunized with stem only HA (the comparison could only be performed for the dose of 0.2 μg). When a full ectodomain HA from B/Yamagata group was used for the coating (FIG. 16), the same trend was observed but only for the groups that had been immunized with wild-type full ectodomain HA. Furthermore, in all the ELISA performed with full ectodomain HA, high antibody responses could be observed in the groups that had been immunized with 2 μg of stem only HA. But the antibody IgG response measured in the recombinant HA groups had the global trend to be lower than the one measured in the QIV adjuvanted group.

A coating with a stem only HA was also used for the assay to better detect anti-stem antibodies. The stem only HA used were expressed on nanoparticles to ensure antigen stability while avoiding the use of a foldon the presence of which could result in the detection of non-Flu but foldon-specific Ab responses. In this assay (FIG. 17), low levels of stem-specific antibody titers were detected for the groups that had been immunized with 0.2 μg of full ectodomain HA, stem only HA or QIV. But slightly higher antibody titers were detected for groups that had been immunized with the dose of 2 μg of stem only HA, with similar titers for the 3 constructs tested at this dose.

### Conclusions for Study B:

For all the B strains tested, the stem only HA with ferritin induced higher antibody titers than the stem only without ferritin, without any impact when the dose was reduced to 0.2 μg and any obvious between-construct difference (FIGS. 18, 19 and 20). The level of antibodies elicited by stem only HA with ferritin was close to the ones elicited by QIV adjuvanted, but remained lower for the B/Washington strain. The level of antibodies induced by stem only without ferritin was not drastically higher than QIV unadjuvanted.

### Example 10—Hi Antibodies at 14 Days Post Dose 2

HI functional antibodies against different B strains from B/Victoria and B/Yamagata groups were measured at 14 days post second immunization (day 42). The results from Study A are shown in FIGS. 21 to 26. Only the groups from study A that received full ectodomain HA and QIV were tested in HI, as this functionality is mediated by the head of the HA. Therefore, the stem only HA antigens, lacking the HA head, are not expected to elicit any HI activity, and were not tested here for HI titers.

In each figure, individual titers values are shown together with the geometric mean titers (GMT) and 95% confidence intervals (95Cl).

### Conclusions:

Low levels (or absence) of HI antibody responses were observed in all HA adjuvanted groups, even for the homologous strain B/Washington/2/2019 (FIGS. 21 to 26). By contrast, the QIV adjuvanted group showed higher HI titers against all strains. Interestingly, the highest HI titers for HA adjuvanted groups in comparison to QIV adjuvanted group were observed against B/Austria/1359417/2021 (FIG. 23), which is the new B/Victoria vaccine strain (2022-23 flu season).

One possible explanation for the low HI titers may be the use of virus propagated in eggs in the HI assay, which may have different glycosylation compared to the full ectodomain constructs.

### Example 11—Neutralizing Antibodies at 14 Days Post Dose 2

Neutralizing antibodies against B/Washington/2/2019 and B/Phuket/3073/2013 were measured at 14 days post second immunization (day 42) for Study A only. The stem only groups were tested only as pooled sera from 8 mice. The results from this assay are not shown in the figures. Values were geometric mean titers (GMT) and 95% confidence interval (95Cl), when applicable.

### Conclusions:

Except for 2 mice in one full ectodomain HA group (Flu209 0.2 μg, with AS03), that had a low titer of 40 against B/Washington/2/2019 strain, there was no detection of neutralizing antibody response in all HA adjuvanted groups. In contrast, high neutralizing antibody titers were detected against both strains in the control group previously immunized with QIV. Once again, a possible explanation for the lack of detection of a neutralizing antibody response could be that the virus used in the assay was egg adapted.

### Example 12—Anti-Stem Antibodies by ADCC Reporter Bioassay at 14 Days Post Dose 2

ADCC activity against B/Washington/2/2019 stem was measured by ADCC Reporter Bioassay Promega at 14 days post second immunization (day 42). The results from Study A are shown in FIG. 27.

Individual AUC (Area under the curve) values are shown together with the geometric mean titers (GMT) and 95% confidence intervals (95Cl).

### Conclusions:

High levels of stem specific functional antibodies were detected for the groups that received the HA stem at a dose of 2 μg adjuvanted, correlating with the high levels of antibodies detected in the ELISA performed with stem antigen for these groups. Low or very variable antibody titers were detected in the other groups.

1. A recombinant influenza B strain haemagglutinin (HA) antigen in trimeric form, comprising one or more mutations.

2. The recombinant HA antigen of claim 1, comprising an HA stem domain in the absence of an HA head domain.

3. (canceled)

4. (canceled)

5.

6. (canceled)

7. (canceled)

8. (canceled)

9. The recombinant HA antigen of claim 1, comprising both an HA stem domain and an HA head domain.

10. (canceled)

11. (canceled)

12. (canceled)

13. (canceled)

14. The recombinant HA antigen claim 1, in which the CR9114 epitope is present.

15. The recombinant HA antigen of claim 1, in which the 5A7 epitope is present.

16. The recombinant HA antigen of claim 1, stabilised in the prefusion conformation.

17. The recombinant HA antigen of claim 1, having at least one stabilising amino acid substitution.

18. The recombinant HA antigen of claim 17, having at least one stabilising amino acid substitution in one or more of Regions A, B and C.

19. The recombinant HA antigen of claim 18, having a stabilising amino acid substitution at position 465 and/or 477.

20. The recombinant HA antigen of claim 17, having at least one stabilising amino acid substitution at one or more of positions 410, 429, 432, 450, 462, 465, 472 and 477.

21. The recombinant HA antigen of claim 17, having at least one stabilising amino acid substitution at one or more of positions 410, 419, 446, 447, 449, 450, 451, 462, 465, and 477.

22. The recombinant HA antigen of claim 1, having an amino acid sequence having at least 85% or at least 87% or at least 90% identity to an amino acid sequence selected from SEQ ID Nos: 2-25 and 84-121 and 160-179, with or without the signal sequence.

23. An isolated polynucleotide such as DNA or mRNA, encoding the recombinant HA antigen of claim 1.

24. The polynucleotide of claim 23 in a nucleic acid delivery platform.

25. An immunogenic composition comprising a recombinant HA, or a polynucleotide encoding a recombinant HA, of claim 1, and pharmaceutically acceptable carrier.

26. (canceled)

27. (canceled)

28. (canceled)

29. The immunogenic composition of claim 25, wherein the recombinant B strain HA is capable of inducing an immune response against at least one additional influenza B strain, wherein the B strains are from the same or different lineages.

30. (canceled)

31. A method of generating an immune response against influenza B strain, comprising administering a recombinant HA antigen or polynucleotide or immunogenic composition of claim 1 to a human subject.

32. (canceled)


SEQUENCE LISTING (179 sequences)

SEQ ID NO: 1 - protein, 582 aa, Influenza B virus
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 2 - protein, 320 aa, Artificial
  Recombinant influenza B haemagglutinin stem - TEV cleavage site - foldon - poly His
MKAIIVLLMVVTSNADRICTGITSSNSPHVVKTATQGEVNVTGVIPLTTTPTKSHFAGSGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNGSGSDLRADTISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 3 - protein, 273 aa, Artificial
  Recombinant influenza B haemagglutinin stem - TEV cleavage site - foldon - poly His
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKNLNSLSELEVKNGPSPGDLRADTISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 4 - protein, 262 aa, Artificial
  Recombinant influenza B haemagglutinin stem - TEV cleavage site - foldon - poly His
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKNLNSLSELEGSGSSSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 5 - protein, 263 aa, Artificial
  Recombinant influenza B haemagglutinin stem - TEV cleavage site - foldon - poly His
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKNLNSLSELEGSGSGSSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 6 - protein, 262 aa, Artificial
  Recombinant influenza B haemagglutinin stem with amino acid substitutions - TEV cleavage site - foldon - poly His
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKMLNSLSELEGSGSSSQIELAVLLVNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 7 - protein, 264 aa, Artificial
  Recombinant influenza B haemagglutinin stem with amino acid substitutions - TEV cleavage site - foldon - poly His
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKMLNSLSELEGSGGSGSSQIELAVLLVNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 8 - protein, 265 aa, Artificial
  Recombinant influenza B haemagglutinin stem with amino acid substitutions - TEV cleavage site - foldon - poly His
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKMLNSLSELEGSGGSGSSQIELAVLLVNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 9 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAPDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 10 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDEPHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 11 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAPDEPHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 12 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKMLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 13 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADVISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 14 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADMISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 15 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEVIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 16 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNELIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 17 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 18 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDIHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 19 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEIIINSEDLHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 20 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 21 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADVISSQIELAVLLSNELIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 22 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADMISSQIELAVLLSNEFIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 23 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKMLNSLSELEVKNLQRLSGAMDELHNEILELDEKVDDLRADMISSQIELAVLLVNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 24 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKMLNSLSELEVKNLQRLSGAMDELHNEILELDEKVDDLRADTISSQIELAVLLVNEGIINSEDVHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 25 - protein, 582 aa, Artificial
  Recombinant influenza B haemagglutinin ectodomain with amino acid substitutions - TEV cleavage site - foldon - poly His
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKMLNSLSELEVKNLQRLSGAMDELHNEILELDEKVDDLRADMISSQIELAVLLVNEGIINSEDVHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 26 - protein, 27 aa, Bacteriophage T4
GYIPEAPRDGQAYVRKDGEWVLLSTFL

SEQ ID NO: 27 - protein, 15 aa, Influenza B virus
MKAIIVLLMVVTSNA

SEQ ID NO: 28 - protein, 23 aa, Influenza B virus
ILLYYSTAASSLAVTLMIAIFVV

SEQ ID NO: 29 - DNA, 966 bp, Artificial
  Nucleotide sequence coding for influenza B haemagglutinin stem - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccggcagcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacggcagcggcagcgacctgagagccgacaccatcagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 30 - DNA, 825 bp, Artificial
  Nucleotide sequence coding for influenza B haemagglutinin stem - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacggccctagccccggcgacctgagagccgacaccatcagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 31 - DNA, 792 bp, Artificial
  Nucleotide sequence coding for influenza B haemagglutinin stem - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggagggcagcggcagcagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 32 - DNA, 795 bp, Artificial
  Nucleotide sequence coding for influenza B haemagglutinin stem - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggagggcagcggcagcggcagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 33 - DNA, 792 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggagggcagcggcagcagcagccaaatcgagctggccgtgctgctggtgaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 34 - DNA, 798 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggagggcagcggcggcagcggcagcagccaaatcgagctggccgtgctgctggtgaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 35 - DNA, 801 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggagggcagcggcggcagcggcagcagccaaatcgagctggccgtgctgctggtgaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 36 - DNA, 1752 bp, Artificial
  Nucleotide sequence coding for influenza B haemagglutinin ectodomain with amino acid substitutions - TEV cleavage site - foldon - poly His
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgcccctgacgaactgcacaacgagatcctcgagctggacgagaaggtggacgacctgagagctgacaccatcagcagccaaattgagctggctgtgctgctgtccaacgaaggcatcatcaacagcgaggacgagcacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 37 - DNA, 1752 bp, Artificial
  Nucleotide sequence coding for influenza B haemagglutinin ectodomain with amino acid substitutions - TEV cleavage site - foldon - poly His
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaaccccacaacgagatcctcgagctggacgagaaggtggacgacctgagagctgacaccatcagcagccaaattgagctggctgtgctgctgtccaacgaaggcatcatcaacagcgaggacgagcacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 38 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgcccctgacgaaccccacaacgagatcctcgagctggacgagaaggtggacgacctgagagctgacaccatcagcagccaaattgagctggctgtgctgctgtccaacgaaggcatcatcaacagcgaggacgagcacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 39 - DNA, 1752 bp, Artificial
  Nucleotide sequence coding for influenza B haemagglutinin ectodomain with amino acid substitutions - TEV cleavage site - foldon - poly His
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagatgctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaggcatcatcaacagcgaggacgagcacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 40 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacgtgatcagcagccaaattgagctggctgtgctgctgtccaacgaaggcatcatcaacagcgaggacgagcacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 41 - DNA, 1752 bp, Artificial; identical to SEQ ID NO: 40
  Nucleotide sequence coding for influenza B haemagglutinin ectodomain with amino acid substitutions - TEV cleavage site - foldon - poly His.

SEQ ID NO: 42 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacctgcagagactgagcggcgccatggacgagctgcacaacgagatcctggagctggacgagaaggtggacgacctgagagccgacaccatcagcagccaaatcgagctggccgtgctgctgagcaacgaggtgatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 43 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacctgcagagactgagcggcgccatggacgagctgcacaacgagatcctggagctggacgagaaggtggacgacctgagagccgacaccatcagcagccaaatcgagctggccgtgctgctgagcaacgagctgatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 44 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacctgcagagactgagcggcgccatggacgagctgcacaacgagatcctggagctggacgagaaggtggacgacctgagagccgacaccatcagcagccaaatcgagctggccgtgctgctgagcaacgagttcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 45 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaggcatcatcaacagcgaggacatccacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 46 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaatcatcatcaacagcgaggacctgcacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 47 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaggcatcatcaacagcgaggacgagcacctgctggcccaagagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 48 - DNA, 1752 bp, Artificial
  Nucleotide sequence coding for influenza B haemagglutinin ectodomain with amino acid substitutions - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacctgcagagactgagcggcgccatggacgagctgcacaacgagatcctggagctggacgagaaggtggacgacctgagagccgacgtgatcagcagccaaatcgagctggccgtgctgctgagcaacgagctgatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 49 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacctgcagagactgagcggcgccatggacgagctgcacaacgagatcctggagctggacgagaaggtggacgacctgagagccgacatgatcagcagccaaatcgagctggccgtgctgctgagcaacgagttcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 50 - DNA, 1752 bp, Artificial
  Nucleotide sequence coding for influenza B haemagglutinin ectodomain with amino acid substitutions - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggaggtgaagaacctgcagagactgagcggcgccatggacgagctgcacaacgagatcctggagctggacgagaaggtggacgacctgagagccgacatgatcagcagccaaatcgagctggccgtgctgctggtgaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 51 - DNA, 1752 bp, Artificial
  Nucleotide sequence coding for influenza B haemagglutinin ectodomain with amino acid substitutions - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggaggtgaagaacctgcagagactgagcggcgccatggacgagctgcacaacgagatcctggagctggacgagaaggtggacgacctgagagccgacaccatcagcagccaaatcgagctggccgtgctgctggtgaacgagggcatcatcaacagcgaggacgtgcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 52 - DNA, 1752 bp, Artificial
  Nucleotide sequence coding for influenza B haemagglutinin ectodomain with amino acid substitutions - TEV cleavage site - foldon - poly His
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgctaccaaccctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggaggtgaagaacctgcagagactgagcggcgccatggacgagctgcacaacgagatcctggagctggacgagaaggtggacgacctgagagccgacatgatcagcagccaaatcgagctggccgtgctgctggtgaacgagggcatcatcaacagcgaggacgtgcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 53 - protein, 4 aa, Artificial
  Linker
GSGS

SEQ ID NO: 54 - protein, 4 aa, Artificial
  Linker
GSPG

SEQ ID NO: 55 - protein, 4 aa, Artificial
  Linker
GPSG

SEQ ID NO: 56 - protein, 5 aa, Artificial
  Linker
GPSPG

SEQ ID NO: 57 - protein, 5 aa, Artificial
  Linker
GSGSG

SEQ ID NO: 58 - protein, 6 aa, Artificial
  Linker
GSGGSG

SEQ ID NO: 59 - protein, 14 aa, Artificial
  Linker
DVANKVSKATDGSG

SEQ ID NO: 60 - protein, 15 aa, Artificial
  Linker
DVANKVSKATDGSGG

SEQ ID NO: 61 - protein, 11 aa, Artificial
  Linker
DVANKVSKAGS

SEQ ID NO: 62 - protein, 11 aa, Artificial
  Linker
DVANKVSKAGG

SEQ ID NO: 63 - protein, 4 aa, Artificial
  Linker
ASTG

SEQ ID NO: 64 - protein, 6 aa, Artificial
  Linker
IPGTGT

SEQ ID NO: 65 - protein, 4 aa, Artificial
  Linker
LPTS

SEQ ID NO: 66 - protein, 5 aa, Artificial
  Linker
YADGG

SEQ ID NO: 67 - protein, 6 aa, Artificial
  Linker
LKGTGG

SEQ ID NO: 68 - protein, 4 aa, Artificial
  Linker
QSGG

SEQ ID NO: 69 - protein, 5 aa, Artificial
  Linker
TPSGS

SEQ ID NO: 70 - protein, 6 aa, Artificial
  Linker
DQTTQT

SEQ ID NO: 71 - protein, 8 aa, Artificial
  Linker
IAGPQGSV

SEQ ID NO: 72 - protein, 9 aa, Artificial
  Linker
LTVNGEDVG

SEQ ID NO: 73 - protein, 10 aa, Artificial
  Linker
VAIPNTSYVV

SEQ ID NO: 74 - protein, 8 aa, Artificial
  Linker
FENNLFRA

SEQ ID NO: 75 - protein, 9 aa, Artificial
  Linker
VAFNNDNNQ

SEQ ID NO: 76 - protein, 10 aa, Artificial
  Linker
STYAAIGNAL

SEQ ID NO: 77 - protein, 10 aa, Artificial
  Linker
SKILTGDYTS

SEQ ID NO: 78 - protein, 11 aa, Artificial
  Linker
SAFYEAFPGAT

SEQ ID NO: 79 - protein, 12 aa, Artificial
  Linker
SATLDEIPSLKE

SEQ ID NO: 80 - protein, 12 aa, Artificial
  Linker
SESFKQTEGQPA

SEQ ID NO: 81 - protein, 16 aa, Artificial
  Linker
SETVAKWEKQLAAGDP

SEQ ID NO: 82 - protein, 17 aa, Artificial
  Linker
SNAMSKLAQQFTDQPTP

SEQ ID NO: 83 - protein, 536 aa, Influenza B virus
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASL

SEQ ID NO: 84 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNELIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 85 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 86 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAPDEPHNEILELDEKVDDLRADTISSQIELAVLLSNELIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 87 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKMLNSLSELEVKNLQRLSGAMDELHNEILELDEKVDDLRADTISSQIELAVLLVNELIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 88 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDIHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 89 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDLHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 90 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEIIINSEDIHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 91 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDIHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 92 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEIIINSEDLHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 93 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDLHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 94 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKMLNSLSELEVKNLQRLSGAPDEPHNEILELDEKVDDLRADTISSQIELAVLLVNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 95 - protein, 582 aa, Artificial
  Recombinant influenza B haemagglutinin ectodomain with amino acid substitutions - TEV cleavage site - foldon - poly His.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKMLNSLSELEVKNLQRLSGAPDEPHNEILELDEKVDDLRADTISSQIELAVLLVNELIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 96 - protein, 273 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKNLNSLSELEVKNGSGSDLRADTISSQIELAVLLSNEVIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 97 - protein, 273 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKNLNSLSELEVKNGSGSDLRADTISSQIELAVLLSNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 98 - protein, 273 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKMLNSLSELEVKNGSGSDLRADTISSQIELAVLLVNEVIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 99 - protein, 273 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKMLNSLSELEVKNGSGSDLRADTISSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 100 - protein, 263 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKMLNSLSELEGSGSGSSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 101 - protein, 265 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKMLNSLSELEGSGGSGSSQIELAVLLVNEFIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 102 - protein, 265 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKMLNSLSELEGSGGSGSSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 103 - protein, 273 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKNLNSLSELEVKNGSGSDSRADVISSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 104 - protein, 273 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKNLNSLSELESKNGSGSDQRADVNSSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 105 - protein, 273 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKNLNSLSELESKNGSGSDQRADVNSSQIELAVLLSNELIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 106 - protein, 264 aa, Artificial
  Recombinant influenza B haemagglutinin stem - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKNLNDVANKVSKAGSSSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 107 - protein, 264 aa, Artificial
  Recombinant influenza B haemagglutinin stem - TEV cleavage site - foldon - poly His.
MKAIIVLLMVVTSNADRICTGITSSNSPHVVKTATQGEVNVTGVIPLGSGLKLANGTKYRPPAKLLKERGFFGAIAGFLEGGWEGMIAGWHGYTSHGAHGVAVAADLKSTQEAINKITKNLNDVANKVSKAGGSSQIELAVLLSNEGIINSEDEHLLALERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 108 - protein, 263 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPASTGKLANGTKYRPPAKLLKERGFFGAIAGFLEGGWEGMIAGWHGYTSHGAHGVAVAADLKSTQEAINKITKMLNSLSKILTGDYTSSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 109 - protein, 265 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPIPGTGTKLANGTKYRPPAKLLKERGFFGAIAGFLEGGWEGMIAGWHGYTSHGAHGVAVAADLKSTQEAINKITKMLNSLSKILTGDYTSSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 110 - protein, 264 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLPTSKLANGTKYRPPAKLLKERGFFGAIAGFLEGGWEGMIAGWHGYTSHGAHGVAVAADLKSTQEAINKITKMLNSLSAFYEAFPGATSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 111 - protein, 265 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPYADGGKLANGTKYRPPAKLLKERGFFGAIAGFLEGGWEGMIAGWHGYTSHGAHGVAVAADLKSTQEAINKITKMLNSLSAFYEAFPGATSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 112 - protein, 266 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLKGTGGKLANGTKYRPPAKLLKERGFFGAIAGFLEGGWEGMIAGWHGYTSHGAHGVAVAADLKSTQEAINKITKMLNSLSAFYEAFPGATSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 113 - protein, 265 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPQSGGKLANGTKYRPPAKLLKERGFFGAIAGFLEGGWEGMIAGWHGYTSHGAHGVAVAADLKSTQEAINKITKMLNSLSATLDRIPSLKESQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 114 - protein, 266 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPTPSGSKLANGTKYRPPAKLLKERGFFGAIAGFLEGGWEGMIAGWHGYTSHGAHGVAVAADLKSTQEAINKITKMLNSLSATLDEIPSLKESQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 115 - protein, 267 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPDQTTQTKLANGTKYRPPAKLLKERGFFGAIAGFLEGGWEGMIAGWHGYTSHGAHGVAVAADLKSTQEAINKITKMLNSLSESFKQTEGQPASQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 116 - protein, 280 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPIAGPQGSVKLANGTKYRPPAKLLKERGFFGAIAGFLEGGWEGMIAGWHGYTSHGAHGVAVAADLKSTQEAINKITKMLNSLSETVAKWEKQLAAGDPSAADTISSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 117 - protein, 281 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPLTVNGEDVGKLANGTKYRPPAKLLKERGFFGAIAGFLEGGWEGMIAGWHGYTSHGAHGVAVAADLKSTQEAINKITKMLNSLSETVAKWEKQLAAGDPSAADTISSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 118 - protein, 282 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPVAIPNTSYVVKLANGTKYRPPAKLLKERGFFGAIAGFLEGGWEGMIAGWHGYTSHGAHGVAVAADLKSTQEAINKITKMLNSLSETVAKWEKQLAAGDPSAADTISSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 119 - protein, 281 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPFENNLFRAKLANGTKYRPPAKLLKERGFFGAIAGFLEGGWEGMIAGWHGYTSHGAHGVAVAADLKSTQEAINKITKMLNSLSNAMSKLAQQFTDQPTPSAADTISSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 120 - protein, 282 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPVAFNNDNNQKLANGTKYRPPAKLLKERGFFGAIAGFLEGGWEGMIAGWHGYTSHGAHGVAVAADLKSTQEAINKITKMLNSLSNAMSKLAQQFTDQPTPSAADTISSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 121 - protein, 283 aa, Artificial
  Recombinant influenza B haemagglutinin stem with amino acid substitutions - TEV cleavage site - foldon - poly His.
MKAIIVLLMVVTSNADRICTGITSSNSPHVVKTATQGEVNVTGVIPSTYAAIGNALKLANGTKYRPPAKLLKERGFFGAIAGFLEGGWEGMIAGWHGYTSHGAHGVAVAADLKSTQEAINKITKMLNSLSNAMSKLAQQFTDQPTPSAADTISSQIELAVLLVNEFIINSEDEHLLAQERKLKKMLGPSAVEIGNGCFETKHKCNQTCLDRIAAGTFDAGEFSLPTFDSLNITAASLGSENLYFQGGSKGYIPEAPRDGQAYVRKDGEWVLLSTFLGHHHHHH

SEQ ID NO: 122 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgctacacaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgccaccaatcctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagcggggcttcttcggggccatcgccggctttctcgagggcggctgggaggggatgatcgctgggtggcacgggtacacaagccatggcgcccacggcgtggccgtggccgccgacctcaagagcacccaagaggccatcaacaagatcaccaagaacctgaacagcctgagcgagctggaggtgaagaacctgcagagactgagcggcgccatggacgagctgcacaacgagatcctggagctggacgagaaggtggacgacctgagagccgacaccatcagcagccaaatcgagctggccgtcctcctgtccaacgagctgatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 123 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgctacacaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgccaccaatcctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagcggggcttcttcggggccatcgccggctttctcgagggcggctgggaggggatgatcgctgggtggcacgggtacacaagccatggcgcccacggcgtggccgtggccgccgacctcaagagcacccaagaggccatcaacaagatcaccaagaacctgaacagcctgagcgagctggaggtgaagaacctgcagagactgagcggcgccatggacgagctgcacaacgagatcctggagctggacgagaaggtggacgacctgagagccgacaccatcagcagccaaatcgagctggccgtcctcctgtccaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 124 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgctacacaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgccaccaatcctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagcggggcttcttcggggccatcgccggctttctcgagggcggctgggaggggatgatcgctgggtggcacgggtacacaagccatggcgcccacggcgtggccgtggccgccgacctcaagagcacccaagaggccatcaacaagatcaccaagaacctgaacagcctgagcgagctggaggtgaagaacctgcagagactgagcggcgcccccgacgagccccacaacgagatcctggagctggacgagaaggtggacgacctgagagccgacaccatcagcagccaaatcgagctggccgtcctcctgtccaacgagctgatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 125 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgctacacaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgccaccaatcctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagcggggcttcttcggggccatcgccggctttctcgagggcggctgggaggggatgatcgctgggtggcacgggtacacaagccatggcgcccacggcgtggccgtggccgccgacctcaagagcacccaagaggccatcaacaagatcaccaagatgctgaacagcctgagcgagctggaggtgaagaacctgcagagactgagcggcgccatggacgagctgcacaacgagatcctggagctggacgagaaggtggacgacctgagagccgacaccatcagcagccaaatcgagctggccgtcctcctggtgaacgagctgatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 126 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaggcatcatcaacagcgaggacatccacctgctggcccaagagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 127 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaggcatcatcaacagcgaggacctgcacctgctggcccaagagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 128 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaatcatcatcaacagcgaggacatccacctgctggcccaagagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 129 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgagttcatcatcaacagcgaggacatccacctgctggcccaagagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 130 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgaaatcatcatcaacagcgaggacctgcacctgctggcccaagagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 131 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagaacctcaacagcctgtccgagctggaagtcaagaacctgcagcggctgagcggcgccatggacgaactgcacaacgagatcctcgagctggacgagaaggtggacgacctgagagctgacaccatcagcagccaaattgagctggctgtgctgctgtccaacgagttcatcatcaacagcgaggacctgcacctgctggcccaagagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 132 - DNA, 1752 bp, Artificial
  Nucleotide sequence coding for influenza B haemagglutinin ectodomain with amino acid substitutions - TEV cleavage site - foldon - poly His.
atgaaggccattatcgtgctcctgatggtggtgacaagcaatgctgacagaatttgcaccgggatcacaagcagcaacagccctcacgtggtgaagaccgccacccaaggcgaggtgaacgtgaccggcgtgatccccctgaccacaacccccaccaagagccacttcgccaacctgaagggcaccgagacaagaggcaagctgtgccccaaatgtctgaattgtaccgatctggacgtggccctgggcagacccaagtgcaccggcaagatccctagcgctagagtgagcatcctgcacgaggtgagacccgtgacatccggctgctttcccattatgcacgaccggaccaagatcagacagctgcccaacctgctgagaggctacgagcacgtgagactgagcacccataacgtgatcaacgccgaggacgcccccggcagaccctacgagatcggcacaagcgggagctgccccaacattaccaacggcaacggcttctttgccaccatggcctgggctgtgcccaagaacaaaaccgctaccaaccccctgaccatcgaggtgccctatatttgcaccgaaggcgaggatcagatcacagtctggggctttcacagcgacaacgagacacagatggccaagctgtacggcgacagcaagcctcagaagttcacaagcagcgccaacggcgtgacaacacactacgtgtcccaaatcggcggcttccccaatcagaccgaagatgggggcctgcctcagagcggccggatcgtggtggactacatggtgcaaaagagcggcaagaccggcaccatcacctatcagagaggcatcctgctgcctcagaaggtgtggtgcgctagcggcagaagcaaagtgatcaaggggtccctccccctgatcggcgaggctgactgcctgcacgagaagtacggcggcctgaacaagagcaagccctactacaccggcgagcatgccaaggccatcggcaactgccccatttgggtcaagacccccctgaagctcgccaacggcaccaaatacagacctcccgccaagctcctgaaggagagaggctttttcggcgccatcgccggcttcctggaggggggctgggaaggcatgatcgccggctggcacggctacacatcccacggcgcccacggcgtggccgtcgccgccgacctcaagagcacccaagaggccatcaacaaaatcaccaagatgctcaacagcctgtccgagctggaagtcaagaacctgcagcggctgagcggcgcccccgacgagccccacaacgagatcctcgagctggacgagaaggtggacgacctgagagctgacaccatcagcagccaaattgagctggctgtgctgctggtgaacgaaggcatcatcaacagcgaggacgagcacctgctggccctggagagaaaactgaaaaagatgctgggcccctccgccgtggagatcggcaacggctgcttcgaaaccaagcacaaatgcaatcaaacatgcctcgaccggatcgccgccggcaccttcgacgccggcgagttcagcctgcccaccttcgacagcctgaacatcaccgctgcctccctcggcagcgagaacctgtacttccaaggcggcagcaagggctacatccccgaggcccctagagacggccaagcctacgtgagaaaggacggcgagtgggtgctgctgtccaccttcctgggccaccatcaccatcaccactgatga

SEQ ID NO: 133 - DNA, 1752 bp, Artificial
  Nucleotide sequence coding for influenza B haemagglutinin ectodomain with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgctacacaaggcgaggtgaacgtgaccggcgtgatccccctgaccacaacccccaccaagagccacttcgccaacctgaagggcaccgagacaagagggaagctgtgccccaagtgcctgaactgcaccgacctggacgtggccctgggcagacccaagtgcaccggcaagatccctagcgctagagtgagcatcctgcacgaggtgagacccgtgacaagcggctgcttccccatcatgcacgacagaaccaagatcagacagctgcccaacctgctgagaggctacgagcacgtgagactgagcacccacaacgtgatcaacgccgaggacgcccccggcagaccctacgagatcggcacaagcggcagctgccccaacatcaccaacggcaacggcttcttcgccaccatggcctgggccgtgcccaagaacaagaccgccaccaatcctctgaccatcgaggtgccctacatctgcaccgagggcgaggatcagatcaccgtgtggggcttccacagcgacaacgagacacagatggccaagctgtacggcgacagcaagcctcagaagttcacaagcagcgccaacggcgtgaccacccactacgtgagccaaatcggcggcttccccaatcagaccgaggacggcggcctgcctcagagcggcagaatcgtggtggactacatggtgcaaaagagcggcaagaccggcaccatcacctatcagagaggcatcctgctgcctcagaaggtgtggtgcgctagcggcagaagcaaggtgatcaagggcagcctgcccctgatcggcgaggccgactgcctgcacgagaagtacggcggcctgaacaagagcaagccctactacaccggcgagcacgccaaggccatcggcaactgccccatctgggtgaagacccccctgaagctggccaacggcaccaagtacagaccccccgccaagctgctgaaggagcggggcttcttcggggccatcgccggctttctcgagggcggctgggaggggatgatcgctgggtggcacgggtacacaagccatggcgcccacggcgtggccgtggccgccgacctcaagagcacccaagaggccatcaacaagatcaccaagatgctgaacagcctgagcgagctggaggtgaagaacctgcagagactgagcggcgcccccgacgagccccacaacgagatcctggagctggacgagaaggtggacgacctgagagccgacaccatcagcagccaaatcgagctggccgtcctcctggtgaacgagctgatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 134 - DNA, 825 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacggcagcggcagcgacctgagagccgacaccatcagcagccaaatcgagctggccgtgctgctgagcaacgaggtgatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 135 - DNA, 825 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacggcagcggcagcgacctgagagccgacaccatcagcagccaaatcgagctggccgtgctgctgagcaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 136 - DNA, 825 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggaggtgaagaacggcagcggcagcgacctgagagccgacaccatcagcagccaaatcgagctggccgtgctgctggtgaacgaggtgatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 137 - DNA, 825 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggaggtgaagaacggcagcggcagcgacctgagagccgacaccatcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 138 - DNA, 795 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggagggcagcggcagcggcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 139 - DNA, 801 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggagggcagcggcggcagcggcagcagccaaatcgagctggccgtgctgctggtgaacgagttcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 140 - DNA, 801 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagatgctgaacagcctgagcgagctggagggcagcggcggcagcggcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 141 - DNA, 825 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggaggtgaagaacggcagcggcagcgacagcagagccgacgtgatcagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 142 - DNA, 825 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggagagcaagaacggcagcggcagcgaccagagagccgacgtgaacagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 143 - DNA, 825 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacagcctgagcgagctggagagcaagaacggcagcggcagcgaccagagagccgacgtgaacagcagccaaatcgagctggccgtgctgctgagcaacgagctgatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 144 - DNA, 798 bp, Artificial
  Nucleotide sequence coding for influenza B haemagglutinin stem - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacgacgtggccaacaaggtgagcaaggccggcagcagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 145 - DNA, 798 bp, Artificial
  Nucleotide sequence coding for influenza B haemagglutinin stem - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgggcagcggcctgaagctggccaacggcaccaagtacagaccccccgccaagctgctgaaggagagagggttcttcggcgccatcgccggcttcctggagggcggctgggaaggcatgatcgctgggtggcacggctacacaagccacggcgctcatggcgtggccgtcgccgccgacctcaagagcacccaagaggccatcaacaagatcaccaagaacctgaacgacgtggccaacaaggtgagcaaggccggcggcagcagccaaatcgagctggccgtgctgctgagcaacgagggcatcatcaacagcgaggacgagcacctgctggccctggagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 146 - DNA, 795 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagacagccacccaaggcgaggtcaacgtcaccggcgtcattcccgctagcaccggcaagctggccaacggcaccaagtacagaccccccgccaagctgctgaaggagagaggcttcttcggcgccattgccggcttcctggaggggggctgggagggcatgatcgccgggtggcatgggtacacaagccatggcgcccatggcgtggccgtcgccgccgacctcaagagcacccaagaggccatcaacaagatcaccaagatgctgaacagcctgagcaagatcctgaccggcgactacacaagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 147 - DNA, 801 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccatccccggcaccggcaccaagctggccaacggcaccaagtacagaccccccgccaagctgctgaaggagagaggcttcttcggcgccatcgccgggttcctcgaagggggctgggagggcatgatcgctggctggcacggctacacaagccacggcgcccatggcgtcgctgtcgccgccgacctcaagagcacccaagaggccatcaacaagatcaccaagatgctgaacagcctgagcaagatcctgaccggcgactacacaagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 148 - DNA, 798 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaagtcaatgtcaccggggtcattcccctgcccacaagcaaactggccaacggcaccaagtacagaccccccgccaagctgctgaaagagcggggcttcttcggcgccattgccggcttcctggaggggggctgggaaggcatgattgctggctggcatggctacacaagccacggcgcccatggcgtcgctgtggccgccgacctcaagagcacccaagaggccatcaacaagatcaccaagatgctgaacagcctgagcgccttctacgaggccttccccggcgccacaagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 149 - DNA, 801 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaagtcaatgtcaccggggtcattccctacgccgacggcggcaaactggccaacggcaccaagtacagaccccccgccaagctgctgaaagagcggggcttcttcggcgccattgccggcttcctggaggggggctgggaaggcatgattgctggctggcatggctacacaagccacggcgcccatggcgtcgctgtggccgccgacctcaagagcacccaagaggccatcaacaagatcaccaagatgctgaacagcctgagcgccttctacgaggccttccccggcgccacaagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 150 - DNA, 804 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaagtcaatgtcaccggggtcattcccctgaagggcaccgggggcaaactggccaacggcaccaagtacagaccccccgccaagctgctgaaagagcggggcttcttcggcgccattgccggcttcctggaggggggctgggaaggcatgattgctggctggcatggctacacaagccacggcgcccatggcgtcgctgtggccgccgacctcaagagcacccaagaggccatcaacaagatcaccaagatgctgaacagcctgagcgccttctacgaggccttccccggcgccacaagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 151 - DNA, 801 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatcccccagagcggcggcaagctggccaacggcaccaagtacagaccccccgccaagctgctgaaggagagaggcttcttcggcgccatcgccgggttcctcgaagggggctgggagggcatgatcgctgggtggcacggctacacatcccacggcgcccatggcgtcgctgtggccgccgacctcaagagcacccaagaggccatcaacaagatcaccaagatgctgaacagcctgagcgccaccctggatagaatcccctccctgaaggagagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctcgatagaatcgccgctggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 152 - DNA, 804 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccacccctagcggcagcaagctggccaacggcaccaagtacagaccccccgccaagctgctgaaggagagaggcttcttcggcgccatcgccgggttcctcgaagggggctgggagggcatgatcgctggctggcacggctacacaagccacggcgcccatggcgtcgctgtcgccgccgacctcaagagcacccaagaggccatcaacaagatcaccaagatgctgaacagcctgagcgccaccctggacgagatccctagcctgaaggagagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 153 - DNA, 855 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccctagcacctacgccgccatcggcaacgccctgaagctggccaacggcaccaagtacagaccccccgccaagctgctgaaggagagaggcttcttcggcgccattgccggcttcctggaggggggctgggaggggatgatcgccggctggcatgggtacacaagccacggcgctcatggcgtggctgtggccgccgacctcaagagcacccaagaggccatcaacaagatcaccaagatgctgaacagcctgagcaacgccatgagcaagctggctcagcagttcaccgatcagcccacccctagcgctgccgacaccatcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 154 - DNA, 846 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccatcgccggcccccaaggcagcgtgaagctggccaacggcaccaagtacagaccccccgccaagctgctgaaggagcggggcttcttcggcgccatcgccggcttcctggagggcggctgggagggcatgattgccggctggcatggctacacaagccacggggcccatggggtggctgtcgccgccgacctcaagagcacccaagaggccatcaacaagatcaccaagatgctgaacagcctgagcgagaccgtggccaaatgggagaagcaactcgccgccggcgaccctagcgccgccgacaccatcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 155 - DNA, 849 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccctgaccgtgaacggcgaggacgtgggcaagctggccaacggcaccaagtacagaccccccgccaagctgctgaaggagcggggcttcttcggcgccatcgccggcttcctggagggcggctgggagggcatgattgccggctggcatggctacacaagccacggggcccatggggtggctgtcgccgccgacctcaagagcacccaagaggccatcaacaagatcaccaagatgctgaacagcctgagcgagaccgtggccaaatgggagaagcaactcgccgccggcgaccctagcgccgccgacaccatcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 156 - DNA, 852 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccccgtggccatccccaacacaagctacgtggtgaagctggccaacggcaccaagtacagaccccccgccaagctgctgaaggagcggggcttcttcggcgccatcgccggcttcctggagggcggctgggagggcatgattgccggctggcatggctacacaagccacggggcccatggggtggctgtcgccgccgacctcaagagcacccaagaggccatcaacaagatcaccaagatgctgaacagcctgagcgagaccgtggccaaatgggagaagcaactcgccgccggcgaccctagcgccgccgacaccatcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 157 - DNA, 849 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccctttcgagaacaacctgttcagagccaagctggccaacggcaccaagtacagaccccccgccaagctgctgaaggagagaggcttcttcggcgccattgccggcttcctggaggggggctgggaggggatgatcgccggctggcatgggtacacaagccacggcgctcatggcgtggctgtggccgccgacctcaagagcacccaagaggccatcaacaagatcaccaagatgctgaacagcctgagcaacgccatgagcaagctggctcagcagttcaccgatcagcccacccctagcgctgccgacaccatcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 158 - DNA, 852 bp, Artificial
  Nucleotide sequence coding for influenza B haemagglutinin stem with amino acid substitutions - TEV cleavage site - foldon - poly His.
atgaaggccatcatcgtgctgctgatggtggtgacaagcaacgccgacagaatctgcaccggcatcacaagcagcaacagcccccacgtggtgaagaccgccacccaaggcgaggtgaacgtgaccggcgtgatccctgtggccttcaacaacgacaacaatcagaagctggccaacggcaccaagtacagaccccccgccaagctgctgaaggagagaggcttcttcggcgccattgccggcttcctggaggggggctgggaggggatgatcgccggctggcatgggtacacaagccacggcgctcatggcgtggctgtggccgccgacctcaagagcacccaagaggccatcaacaagatcaccaagatgctgaacagcctgagcaacgccatgagcaagctggctcagcagttcaccgatcagcccacccctagcgctgccgacaccatcagcagccaaatcgagctggccgtgctgctggtgaacgagttcatcatcaacagcgaggacgagcacctgctggcccaagagagaaagctgaagaagatgctgggccctagcgccgtggagatcgggaacggctgcttcgagaccaagcacaagtgcaatcagacctgcctggacagaatcgccgccggcaccttcgacgccggcgagttcagcctgcccaccttcgacagcctgaacatcaccgccgctagcctgggcagcgagaacctgtacttccaaggcggcagcaagggctacatccccgaggcccctagagacggccaagcctacgtgagaaaggacggcgagtgggtgctgctgagcaccttcctgggccaccatcaccatcaccactgatga

SEQ ID NO: 159 - DNA, 855 bp, Artificial; identical to SEQ ID NO: 153
  Nucleotide sequence coding for influenza B haemagglutinin stem with amino acid substitutions - TEV cleavage site - foldon - poly His.

SEQ ID NO: 160 - protein, 262 aa, Artificial
  Recombinant influenza B haemagglutinin stem construct with amino acid substitutions Flu075 - membrane anchoring sequence
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKMLNSLSELEGSGSSSQIELAVLLVNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 161 - protein, 382 aa, Artificial
  Recombinant influenza B haemagglutinin stem construct with amino acid substitutions Flu075 - SGG linker - Helicobacter pylori ferritin
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKMLNSLSELEGSGSSSQIELAVLLVNEGIINSEDEHLLALERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 162 - protein, 264 aa, Artificial
  Recombinant influenza B haemagglutinin stem construct with amino acid substitutions Flu077 - membrane anchoring sequence
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKMLNSLSELEGSGGSGSSQIELAVLLVNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 163 - protein, 384 aa, Artificial
  Recombinant influenza B haemagglutinin stem construct with amino acid substitutions Flu077 - SGG linker - Helicobacter pylori ferritin
MKAIIVLLMVVTSNADRICTGITSSNSPHVVKTATQGEVNVTGVIPLGSLKLANGTKYRPPAKLLKERGFFGAIAGFLEGGWEGMIAGWHGYTSHGAHGVAVAADLKSTQEAINKITKMLNSLSELEGSGGSGSSQIELAVLLVNEGIINSEDEHLLALERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 164 - protein, 582 aa, Artificial
  Recombinant influenza B haemagglutinin ectodomain with amino acid substitutions Flu115 - membrane anchoring sequence
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 165 - protein, 702 aa, Artificial
  Recombinant influenza B haemagglutinin ectodomain with amino acid substitutions Flu115 - SGG linker - Helicobacter pylori ferritin
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDEHLLALERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 166 - protein, 582 aa, Artificial
  Recombinant influenza B haemagglutinin ectodomain with amino acid substitutions Flu159 - membrane anchoring sequence
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLAQERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 167 - protein, 702 aa, Artificial
  Recombinant influenza B haemagglutinin ectodomain with amino acid substitutions Flu159 - SGG linker - Helicobacter pylori ferritin
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLAQERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 168 - protein, 265 aa, Artificial
  Recombinant influenza B haemagglutinin stem construct with amino acid substitutions Flu460 - membrane anchoring sequence
MKAIIVLLMVVTSNADRICTGITSSNSPHVVKTATQGEVNVTGVIPIPGTGTKLANGTKYRPPAKLLKERGFFGAIAGFLEGGWEGMIAGWHGYTSHGAHGVAVAADLKSTQEAINKITKMLNSLSKILTGDYTSSQIELAVLLVNEFIINSEDEHLLAQERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 169 - protein, 385 aa, Artificial
  Recombinant influenza B haemagglutinin stem construct with amino acid substitutions Flu460 - SGG linker - Helicobacter pylori ferritin
MKAIIVLLMVVTSNADRICTGITSSNSPHVVKTATQGEVNVTGVIPIPGTGTKLANGTKYRPPAKLLKERGFFGAIAGFLEGGWEGMIAGWHGYTSHGAHGVAVAADLKSTQEAINKITKMLNSLSKILTGDYTSSQIELAVLLVNEFIINSEDEHLLAQERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 170 - protein, 265 aa, Artificial
  Recombinant influenza B haemagglutinin stem construct with amino acid substitutions Flu474 - membrane anchoring sequence
MKAIIVLLMVVTSNADRICTGITSSNSPHVVKTATQGEVNVTGVIPYADGGKLANGTKYRPPAKLLKERGFFGAIAGFLEGGWEGMIAGWHGYTSHGAHGVAVAADLKSTQEAINKITKMLNSLSAFYEAFPGATSQIELAVLLVNEFIINSEDEHLLAQERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 171 - protein, 385 aa, Artificial
  Recombinant influenza B haemagglutinin stem construct with amino acid substitutions Flu474 - SGG linker - Helicobacter pylori ferritin
MKAIIVLLMVVTSNADRICTGITSSNSPHVVKTATQGEVNVTGVIPYADGGKLANGTKYRPPAKLLKERGFFGAIAGFLEGGWEGMIAGWHGYTSHGAHGVAVAADLKSTQEAINKITKMLNSLSAFYEAFPGATSQIELAVLLVNEFIINSEDEHLLAQERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 172 - protein, 265 aa, Artificial
  Recombinant influenza B haemagglutinin stem construct with amino acid substitutions Flu490 - membrane anchoring sequence
MKAIIVLLMVVTSNADRICTGITSSNSPHVVKTATQGEVNVTGVIPQSGGKLANGTKYRPPAKLLKERGFFGAIAGFLEGGWEGMIAGWHGYTSHGAHGVAVAADLKSTQEAINKITKMLNSLSATLDRIPSLKESQIELAVLLVNEFIINSEDEHLLAQERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 173 - protein, 385 aa, Artificial
  Recombinant influenza B haemagglutinin stem construct with amino acid substitutions Flu490 - SGG linker - Helicobacter pylori ferritin
MKAIIVLLMVVTSNADRICTGITSSNSPHVVKTATQGEVNVTGVIPQSGGKLANGTKYRPPAKLLKERGFFGAIAGFLEGGWEGMIAGWHGYTSHGAHGVAVAADLKSTQEAINKITKMLNSLSATLDRIPSLKESQIELAVLLVNEFIINSEDEHLLAQERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 174 - protein, 282 aa, Artificial
  Recombinant influenza B haemagglutinin stem construct with amino acid substitutions Flu523 - membrane anchoring sequence
MKAIIVLLMVVTSNADRICTGITSSNSPHVVKTATQGEVNVTGVIPVAIPNTSYVVKLANGTKYRPPAKLLKERGFFGAIAGFLEGGWEGMIAGWHGYTSHGAHGVAVAADLKSTQEAINKITKMLNSLSETVAKWEKQLAAGDPSAADTISSQIELAVLLVNEFIINSEDEHLLAQERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 175 - protein, 402 aa, Artificial
  Recombinant influenza B haemagglutinin stem construct with amino acid substitutions Flu523 - SGG linker - Helicobacter pylori ferritin
MKAIIVLLMVVTSNADRICTGITSSNSPHVVKTATQGEVNVTGVIPVAIPNTSYVVKLANGTKYRPPAKLLKERGFFGAIAGFLEGGWEGMIAGWHGYTSHGAHGVAVAADLKSTQEAINKITKMLNSLSETVAKWEKQLAAGDPSAADTISSQIELAVLLVNEFIINSEDEHLLAQERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 176 - protein, 582 aa, Artificial
  Recombinant influenza B haemagglutinin ectodomain with amino acid substitutions Flu209 - membrane anchoring sequence
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNELIINSEDEHLLAQERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 177 - protein, 702 aa, Artificial
  Recombinant influenza B haemagglutinin ectodomain with amino acid substitutions Flu209 - SGG linker - Helicobacter pylori ferritin
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNELIINSEDEHLLAQERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 178 - protein, 582 aa, Artificial
  Recombinant influenza B haemagglutinin ectodomain with amino acid substitutions Flu256 - membrane anchoring sequence
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDIHLLAQERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 179 - protein, 702 aa, Artificial
  Recombinant influenza B haemagglutinin ectodomain with amino acid substitutions Flu256 - SGG linker - Helicobacter pylori ferritin
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEFIINSEDIHLLAQERKLKKMLGPSAVEIGNGCFETKHKCNQTCLDRIAAGTFDAGEFSLPTFDSLNITAASLSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS